(19) United States
(12) Patent Application Publication
(10) Pub. No.: US 2023/0276700 A1
(43) Pub. Date: Aug. 31, 2023

(54) Light-Emitting Device, Light-Emitting Apparatus, Electronic Device, and Lighting Device

(71) Applicant: Semiconductor Energy Laboratory Co., Ltd., Kanagawa-ken (JP)

(72) Inventors: Yuta Kawano, Yokohama, Kanagawa (JP); Airi UEDA, Zama, Kanagawa (JP); Takeyoshi WATABE, Atsugi, Kanagawa (JP); Nobuharu OHSAWA, Zama, Kanagawa (JP); Keito TOSU, Atsugi, Kanagawa (JP); Harue OSAKA, Atsugi, Kanagawa (JP); Satoshi SEO, Sagamihara, Kanagawa (JP)

(73) Assignee: Semiconductor Energy Laboratory Co., Ltd., Kanagawa-ken (JP)

(21) Appl. No.: 18/020,490

(22) PCT Filed: Jul. 29, 2021
(86) PCT No.: PCT/IB2021/056887

§ 371 Date: Feb. 9, 2023

(30) Foreign Application Priority Data
Aug. 12, 2020 (JP) 2020-136296

(51) Int. Cl. H10K 85/60; H10K 50/858; H10K 71/13; C07D 251/24; C09K 11/06

(52) U.S. Cl. CPC H10K 85/654; H10K 50/858; H10K 71/135; C07D 251/24; C09K 11/06; H10K 50/15

(57) ABSTRACT

An inexpensive light-emitting device with high emission efficiency is provided. A light-emitting device including an anode, a cathode, and an EL layer positioned between the anode and the cathode is provided. The EL layer includes a hole-transport region, a light-emitting layer, and an electron-transport region. The hole-transport region is positioned between the anode and the light-emitting layer. The electron-transport region is positioned between the cathode and the light-emitting layer. The hole-transport region contains a heteropoly acid and an organic compound having a π-electron rich aromatic ring or contains a heteropoly acid and an organic compound having a π-electron rich heteroaromatic ring. The electron-transport region contains an organic compound with an electron-transport property. The ordinary refractive index of the organic compound with an electron-transport property is higher than or equal to 1.50 and lower than or equal to 1.75 for light with a wavelength of greater than or equal to 455 nm and less than or equal to 465 nm.

### TECHNICAL FIELD

One embodiment of the present invention relates to an organic compound, a light-emitting element, a light-emitting device, a display module, a lighting module, a display device, a light-emitting apparatus, an electronic apparatus, a lighting device, and an electronic device. Note that one embodiment of the present invention is not limited to the above technical field. The technical field of one embodiment of the invention disclosed in this specification and the like relates to an object, a method, or a manufacturing method. One embodiment of the present invention relates to a process, a machine, manufacture, or a composition of matter. Thus, specific examples of the technical field of one embodiment of the present invention disclosed in this specification include a semiconductor device, a display device, a liquid crystal display device, a light-emitting apparatus, a lighting device, a power storage device, a memory device, an imaging device, a driving method thereof, and a manufacturing method thereof.

### BACKGROUND ART

Light-emitting devices (organic EL devices) including organic compounds and utilizing electroluminescence (EL) have been put to more practical use. In the basic structure of such light-emitting devices, an organic compound layer containing a light-emitting material (an EL layer) is sandwiched between a pair of electrodes. Carriers are injected by application of a voltage to the device, and light emission from the light-emitting material can be obtained by using recombination energy of the carriers.

Such light-emitting devices are of self-luminous type and thus have advantages over liquid crystal devices, such as high visibility and no need for a backlight when used for pixels of a display, and are particularly suitable for flat panel displays. Displays including such light-emitting devices are also highly advantageous in that they can be thin and lightweight. Moreover, such light-emitting devices also have a feature that the response speed is extremely fast.

Since light-emitting layers of such light-emitting devices can be successively formed two-dimensionally, planar light emission can be achieved. This feature is difficult to realize with point light sources typified by incandescent lamps or LEDs or linear light sources typified by fluorescent lamps; thus, such light-emitting devices also have a great potential as planar light sources which can be applied to lighting and the like.

Displays or lighting devices including light-emitting devices are suitable for a variety of electronic apparatuses as described above, and research and development of light-emitting devices have progressed for more favorable characteristics.

Low outcoupling efficiency is often a problem in an organic EL device. In particular, the attenuation due to reflection which is caused by a difference in refractive index between adjacent layers is a main cause of a reduction in efficiency of a light-emitting device. In order to reduce this effect, a structure including a layer formed using a low refractive index material in an EL layer (see Patent Document 1, for example) has been proposed.

Commercialized organic EL devices are often manufactured by an evaporation method, which is costly in maintaining material efficiency and the manufacturing atmosphere, for example; thus, employing a wet film formation method is expected to enable inexpensive manufacture.

### REFERENCE

### Patent Document

[Patent Document 1] United States Patent Application Publication No. 2020/0176692

### SUMMARY OF THE INVENTION

### Problems to be Solved by the Invention

An object of one embodiment of the present invention is to provide a light-emitting device with high emission efficiency. Another object of one embodiment of the present invention is to provide any of a light-emitting device, a light-emitting apparatus, an electronic apparatus, a display device, and an electronic device each with low power consumption. Another object of one embodiment of the present invention is to provide an inexpensive light-emitting device. Another object of one embodiment of the present invention is to provide an inexpensive light-emitting device with high emission efficiency.

It is only necessary that any one of the above-described objects be achieved in the present invention.

### Means for Solving the Problems

One embodiment of the present invention is a light-emitting device which includes an anode, a cathode, and an EL layer positioned between the anode and the cathode and in which the EL layer includes a hole-transport region, a light-emitting layer, and an electron-transport region; the hole-transport region is positioned between the anode and the light-emitting layer; the electron-transport region is positioned between the cathode and the light-emitting layer; the hole-transport region includes a layer formed by applying and baking varnish containing a heteropoly acid; the electron-transport region contains an organic compound with an electron-transport property; and the ordinary refractive index of the organic compound with an electron-transport property is higher than or equal to 1.50 and lower than or equal to 1.75 for light with a wavelength of greater than or equal to 455 nm and less than or equal to 465 nm.

Another embodiment of the present invention is a light-emitting device which includes an anode, a cathode, and an EL layer positioned between the anode and the cathode and in which the EL layer includes a hole-transport region, a light-emitting layer, and an electron-transport region; the hole-transport region is positioned between the anode and the light-emitting layer; the electron-transport region is positioned between the cathode and the light-emitting layer; the hole-transport region includes a layer formed by applying and baking varnish containing a heteropoly acid; the electron-transport region contains an organic compound with an electron-transport property; and the ordinary refractive index of the organic compound with an electron-transport property is higher than or equal to 1.45 and lower than or equal to 1.70 for light with a wavelength of 633 nm.

In the above structure, it is preferable that the hole-transport region include a layer formed by applying and baking either varnish containing the heteropoly acid and an organic compound having a π-electron rich aromatic ring or varnish containing the heteropoly acid and an organic compound having a π-electron rich heteroaromatic ring.

In the above structure, it is preferable that the organic compound having a π-electron rich aromatic ring and the organic compound having a π-electron rich heteroaromatic ring be each an aryl amine.

Another embodiment of the present invention is a light-emitting device which includes an anode, a cathode, and an EL layer positioned between the anode and the cathode and in which the EL layer includes a hole-transport region, a light-emitting layer, and an electron-transport region; the hole-transport region is positioned between the anode and the light-emitting layer; the electron-transport region is positioned between the cathode and the light-emitting layer; the hole-transport region contains a heteropoly acid; the electron-transport region contains an organic compound with an electron-transport property; and the ordinary refractive index of the organic compound with an electron-transport property is higher than or equal to 1.50 and lower than or equal to 1.75 for light with a wavelength of greater than or equal to 455 nm and less than or equal to 465 nm.

Another embodiment of the present invention is a light-emitting device which includes an anode, a cathode, and an EL layer positioned between the anode and the cathode and in which the EL layer includes a hole-transport region, a light-emitting layer, and an electron-transport region; the hole-transport region is positioned between the anode and the light-emitting layer; the electron-transport region is positioned between the cathode and the light-emitting layer; the hole-transport region contains a heteropoly acid; the electron-transport region contains an organic compound with an electron-transport property; and the ordinary refractive index of the organic compound with an electron-transport property is higher than or equal to 1.45 and lower than or equal to 1.70 for light with a wavelength of 633 nm.

In the above structure, it is preferable that the hole-transport region contain the heteropoly acid and an organic compound having a π-electron rich aromatic ring or contain the heteropoly acid and an organic compound having a π-electron rich heteroaromatic ring.

In the above structure, it is preferable that the organic compound having a π-electron rich aromatic ring and the organic compound having a π-electron rich heteroaromatic ring be each an aryl amine.

In the above structure, it is preferable that the hole-transport region contain the heteropoly acid and a tertiary amine.

In the above structure, it is preferable that the heteropoly acid be phosphotungstic acid.

In the above structure, it is preferable that the hole-transport region contain an arylsulfonic acid.

In the above structure, it is preferable that the light-emitting layer contain an iridium complex.

In the above structure, it is preferable that the iridium complex emit green phosphorescent light.

In the above structure, it is preferable that the light-emitting layer exhibit a signal at around m/z=1676 in measurement by ToF-SIMS in a positive mode.

In the above structure, it is preferable that the iridium complex be an iridium complex represented by Structural Formula (301) below.

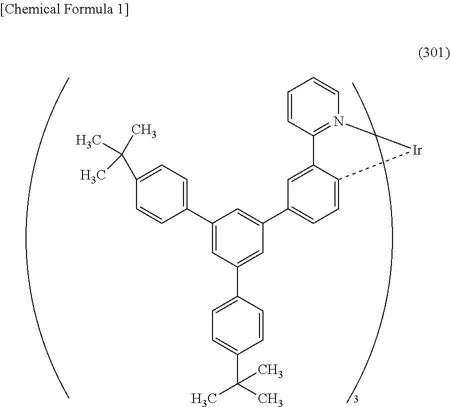

In the above structure, it is preferable that the organic compound with an electron-transport property include at least one nitrogen-containing six-membered heteroaromatic ring, two benzene rings, one or more aromatic hydrocarbon rings each having 6 to 14 carbon atoms, and a plurality of hydrocarbon groups forming a bond by sp³ hybrid orbitals, and the total number of carbon atoms forming the bond by the sp³ hybrid orbitals account for higher than or equal to 10% and lower than or equal to 60% of the total number of carbon atoms in a molecule of the organic compound with an electron-transport property.

In the above structure, it is preferable that the electron-transport region include an electron-transport layer and an electron-injection layer, the electron-injection layer be provided in contact with the cathode, and the electron-transport layer contain the organic compound with an electron-transport property.

In the above structure, it is preferable that the electron-transport layer contain a metal complex of an alkali metal or an alkaline earth metal.

In the above structure, it is preferable that the metal complex of an alkali metal or an alkaline earth metal have a ligand including an 8-quinolinolato structure.

In the above structure, it is preferable that the metal complex of an alkali metal or an alkaline earth metal be a metal complex of lithium.

In the above structure, it is preferable that the electron-injection layer exhibit a signal at around m/z=587 in measurement by ToF-SIMS in a positive mode or a negative mode.

In the above structure, it is preferable that the electron-injection layer contain a heteroaromatic compound.

In the above structure, it is preferable that the heteroaromatic compound be 2-phenyl-9-[3-(9-phenyl-1,10-phenanthrolin-2-yl)phenyl]-1,10-phenanthroline.

In the above structure, it is preferable that the electron-injection layer contain fluorine and sodium.

In the above structure, it is preferable that the electron-injection layer contain barium.

Another embodiment of the present invention is a light-emitting apparatus which includes a plurality of the light-emitting devices having any one of the above-described structures and in which the plurality of light-emitting devices include at least a light-emitting device emitting red light and a light-emitting device emitting green light; and a light-emitting layer of the light-emitting device emitting red light and a light-emitting layer of the light-emitting device emitting green light each contain iridium.

In the above structure, it is preferable that light obtained from the light-emitting device emitting red light and light obtained from the light-emitting device emitting green light be each phosphorescent light.

In the above structure, it is preferable that the plurality of light-emitting devices include a light-emitting device emitting blue light and light obtained from the light-emitting device emitting blue light be fluorescent light.

Another embodiment of the present invention is a light-emitting apparatus including a plurality of the light-emitting devices having any one of the above-described structures.

Another embodiment of the present invention is a display device including the light-emitting apparatus having any one of the above-described structures.

Another embodiment of the present invention is an electronic apparatus including the light-emitting device having any of the above-described structures, a sensor, an operation button, and a speaker or a microphone.

Another embodiment of the present invention is a lighting device including the light-emitting device having any one of the above-described structures and a housing.

Note that the light-emitting apparatus in this specification covers an image display device that includes a light-emitting device. The light-emitting apparatus may also include a module in which a light-emitting device is provided with a connector such as an anisotropic conductive film or a TCP (Tape Carrier Package), a module in which a printed wiring board is provided at the end of a TCP, or a module in which an IC (integrated circuit) is directly mounted on a light-emitting device by a COG (Chip On Glass) method. Furthermore, alighting device or the like may include the light-emitting apparatus.

### Effect of the Invention

One embodiment of the present invention can provide a light-emitting device with high emission efficiency. One embodiment of the present invention can provide any of a light-emitting device, a light-emitting apparatus, an electronic apparatus, a display device, and an electronic device each with low power consumption.

Another embodiment of the present invention can provide a novel organometallic complex (metal complex). Another embodiment of the present invention can provide a metal complex applicable to a light-emitting device with a low driving voltage. Another embodiment of the present invention can provide a metal complex applicable to a light-emitting device including an electron-transport layer with a low refractive index and having a low driving voltage.

Note that the description of these effects does not preclude the existence of other effects. One embodiment of the present invention does not need to have all of these effects. Other effects will be apparent from and can be derived from the description of the specification, the drawings, the claims, and the like.

### BRIEF DESCRIPTION OF THE DRAWINGS

FIG. 1A to FIG. 1D are schematic diagrams of light-emitting devices.

FIG. 2A and FIG. 2B are diagrams showing an active matrix light-emitting apparatus.

FIG. 3A and FIG. 3B are diagrams showing an active matrix light-emitting apparatus.

FIG. 4 is a diagram showing an active matrix light-emitting apparatus.

FIG. 5A and FIG. 5B are diagrams showing a passive matrix light-emitting apparatus.

FIG. 6A and FIG. 6B are diagrams showing a lighting device.

FIG. 7A, FIG. 7B1, FIG. 7B2, and FIG. 7C are diagrams showing electronic apparatuses.

FIG. 8A to FIG. 8C are diagrams showing electronic apparatuses.

FIG. 9 is a diagram showing a lighting device.

FIG. 10 is a diagram showing a lighting device.

FIG. 11 is a diagram showing in-vehicle display devices and lighting devices.

FIG. 12A and FIG. 12B are diagrams showing an electronic apparatus.

FIG. 13A to FIG. 13C are diagrams showing an electronic apparatus.

FIG. 14 shows an absorption spectrum and an emission spectrum of Li-6mq in a dehydrated acetone solution.

FIG. 15 shows measurement data of refractive indices of mmtBumBPTzn.

FIG. 16A to FIG. 16D are diagrams showing an example of a fabrication method of a light-emitting device.

FIG. 17 is a conceptual diagram illustrating a droplet discharge apparatus.

FIG. 18 is a diagram illustrating ESR spectra of Sample 1, Comparative Sample 1, and Comparative Sample 2.

FIG. 19 is a diagram illustrating an ultraviolet-visible absorption spectrum of Sample 1.

FIG. 20 is a diagram illustrating an ultraviolet-visible absorption spectrum of Comparative Sample 1.

FIG. 21 is a diagram illustrating an ultraviolet-visible absorption spectrum of Comparative Sample 2.

### MODE FOR CARRYING OUT THE INVENTION

Embodiments of the present invention will be described in detail below with reference to the drawings. Note that the present invention is not limited to the following description, and it will be readily appreciated by those skilled in the art that modes and details of the present invention can be modified in various ways without departing from the spirit and scope of the present invention. Therefore, the present invention should not be construed as being limited to the description in the following embodiments.

### Embodiment 1

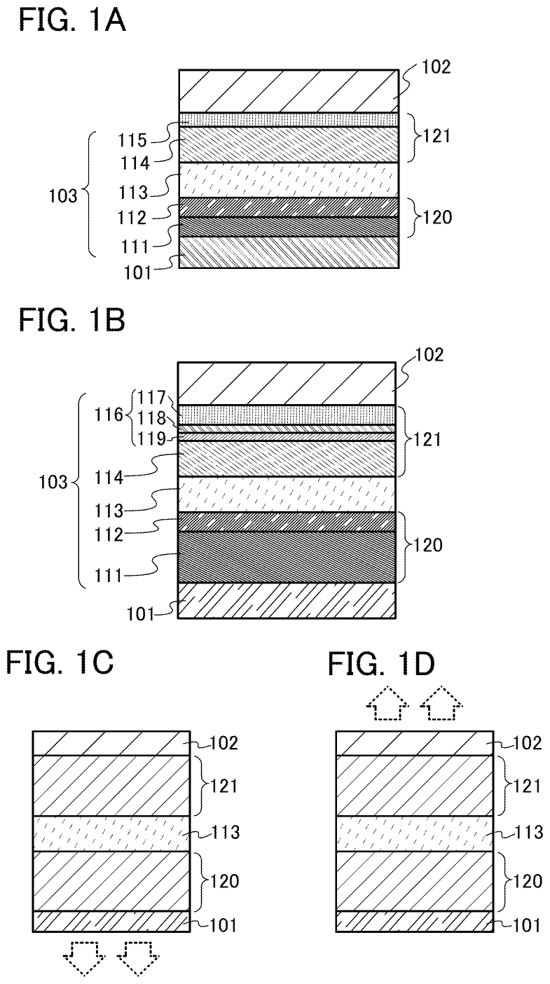

FIG. 1A is a diagram illustrating a light-emitting device of one embodiment of the present invention. The light-emitting device of one embodiment of the present invention includes an anode 101, a cathode 102, and an EL layer 103. The EL layer 103 includes a hole-transport region 120, a light-emitting layer 113, and an electron-transport region 121.

A hole-injection layer 111 and a hole-transport layer 112 are illustrated in the hole-transport region 120, and an electron-transport layer 114 and an electron-injection layer 115 are illustrated in the electron-transport region 121. However, either of the hole-injection layer 111 or the hole-transport layer 112 is not necessarily provided, or either of the electron-transport layer 114 or the electron-injection layer 115 is not necessarily provided. Alternatively, a different functional layer may be provided. Examples of the different functional layer include a carrier-blocking layer, an exciton-blocking layer, and a charge-generation layer.

The light-emitting layer 113 contains at least a light-emitting material and the electron-transport region 121 contains at least an organic compound having an electron-transport property. At least part of the hole-transport region 120 includes a layer formed by a wet film formation method.

The hole-transport region 120 includes a layer formed by depositing ink containing a material by a wet film formation method typified by an inkjet method. The hole-transport region 120 is formed of one layer or a stack of a plurality of layers among layers having desired functions, such as the hole-injection layer 111, the hole-transport layer 112, and an electron-blocking layer. Note that a structure provided with a layer having a plurality of functions such as a hole injection/transport layer may be employed instead of a structure in which one layer has one function.

As it is named, the hole-transport region 120 has a function of transporting holes between the anode 101 and the light-emitting layer 113; thus, the hole-transport region 120 preferably contains a material with a hole-transport property, and further preferably contains a material with a hole-transport property and a material with an acceptor property. The material with a hole-transport property is preferably an organic compound having a π-electron rich aromatic ring or an organic compound having a π-electron rich heteroaromatic ring, for example. An example of the π-electron rich aromatic ring is an aryl amine skeleton. Examples of the π-electron rich heteroaromatic ring include a thiophene skeleton, a pyrrole skeleton, and a carbazole skeleton. Therefore, examples of the material with a hole-transport property include an aryl amine having an aryl amine skeleton, a thiophene derivative having a thiophene skeleton, a pyrrole derivative having a pyrrole skeleton, and a carbazole derivative having a carbazole skeleton. Note that the material with a hole-transport property may be any of a low molecular weight material, an oligomer, a high molecular weight material, and a mixture of these.

In FIG. 1A and FIG. 1B, the hole-transport region 120 includes two layers of the hole-injection layer 111 and the hole-transport layer 112. In the case where the layer in contact with the anode 101 such as the hole-injection layer 111 or a hole injection/transport layer is formed by a wet film formation method, a heteropoly acid (also referred to as a heteropolyoxometalate or a polyoxometalate) can be given as examples of the above material with an acceptor property. The heteropoly acid is preferable because it can be easily deposited by a wet process. The heteropoly acid is preferable because it has high acidity also in a solid state.

A heteropoly acid is an inorganic oxyacid formed by condensation or polymerization of two or more kinds of inorganic oxyacids. A heteropoly acid is represented by [X_{w}MₓO_{y}]ⁿ⁻, for example, where X represents a heteroatom, M represents a polyatom, O represents oxygen, and w, x, y, and n each represent a freely selected integer. Examples of a heteroatom of a heteropoly acid include phosphorus (P), silicon (Si), arsenic (As), germanium (Ge), and boron (B). Examples of an addenda atom (also referred to as a polyatom) of a heteropoly acid include tungsten (W), molybdenum (Mo), vanadium (V), niobium (Nb), and tantalum (Ta). Note that the addenda atom includes one or more of the above elements.

Specific examples of a heteropoly acid include phosphotungstic acid, phosphomolybdic acid, phosphomolybdotungstic acid, phosphomolybdovanadic acid, silicotungstic acid, silicomolybdic acid, germanotungstic acid, germanomolybdic acid, and their compounds. The heteropoly acid used for the hole-transport region 120 is preferably phosphotungstic acid or phosphomolybdic acid. Note that the hole-transport region 120 may include one kind of heteropoly acid or a combination of two or more kinds of heteropoly acids. In particular, a heteropoly acid containing molybdenum, tungsten, or vanadium, which has high oxidizability in addition to high acidity, is preferred because it facilitates generation of carriers to improve conductivity when mixed with an organic compound having a π-electron rich aromatic ring or an organic compound having a π-electron rich heteroaromatic ring. Phosphotungstic acid or phosphomolybdic acid is particularly preferable because it is inexpensive and easy to handle; phosphotungstic acid is further preferable because it does not easily cause coloring.

As primary structures of heteropoly acids, there are a Keggin-type primary structure, a Dawson-type primary structure, and the like. The structure of Keggin-type phosphotungstic acid (H₃PW₁₂O₄₀) is shown below. Note that the framework structure of a polyanion ([PW₁₂O₄₀]³⁻) is shown, whereas a counter cation (which sometimes contains crystal water or an organic molecule) is not shown.

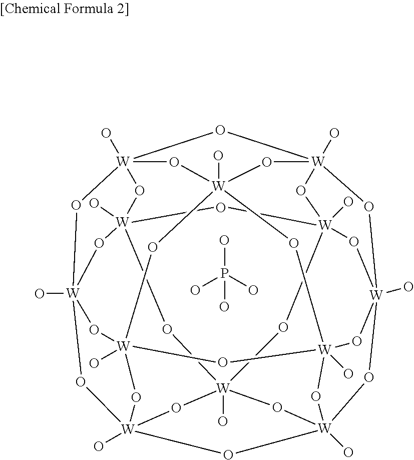

As ink to be applied, a material such as a polymer material, a low molecular weight material, or a dendrimer having a desired function is used as it is or after being dispersed or dissolved in a solvent. Alternatively, ink in which one or more kinds of monomers for a target polymer material are mixed may be applied and heating, energy light irradiation, or the like may be subsequently performed to form a bond such as cross-linking, condensation, polymerization, coordination, or a salt. Note that the ink may contain an organic compound having a different function such as a surface active agent or a substance for adjusting viscosity.

In the case where ink in which monomers are mixed is applied, a secondary aryl amine and a heteropoly acid are preferably used as the monomers. A secondary aryl amine is preferred because it forms cross-linkage by being mixed with a heteropoly acid to improve film quality. In addition, a secondary aryl amine is preferred because it facilitates generation of carriers to improve conductivity.

The aryl amine used as the monomer may be any of a primary amine, a secondary amine, and a tertiary amine but is preferably a tertiary amine. A tertiary amine is electrochemically and photochemically stable as compared to a secondary amine and thereby enables a favorable hole-transport property when mixed with a heteropoly acid. The aryl amine used as the monomer is not limited to one kind of aryl amine but may be a combination of a plurality of aryl amines. For example, it is possible to use both a tertiary amine and a secondary amine, both a primary amine and a tertiary amine, or both a primary amine and a secondary amine. Moreover, all of a primary amine, a secondary amine, and a tertiary amine may be used in combination.

The aryl amine preferably includes a substituted or unsubstituted aryl group having 6 to 14 carbon atoms or a substituted or unsubstituted π-electron rich heteroaryl group having 6 to 12 carbon atoms. Examples of the aryl group include a phenyl group, a biphenyl group, a naphthyl group, a fluorenyl group, a phenanthrenyl group, and an anthryl group. It is particularly preferable to use a phenyl group because it enables favorable solubility of the aryl amine and lowers the raw material price. Examples of the π-electron rich heteroaryl group include a carbazole skeleton, a pyrrole skeleton, a thiophene skeleton, a furan skeleton, and an imidazole skeleton.

The aryl amine preferably includes a plurality of bonds formed via the π-electron rich heteroaryl amine, in which case the film quality is improved. Moreover, an oligomer or a polymer may be formed, which includes many of the bonds. In the case where the aryl amine has a plurality of amine skeletons, some of the amine skeletons may be tertiary amines, secondary amines, or primary amines; the proportion of tertiary amines is preferably high and it is further preferable that all the amine skeletons be tertiary amines to achieve high chemical stability. The number of amine skeletons is preferably less than or equal to 1000, further preferably less than or equal to 10, and the molecular weight is preferably less than or equal to 100000. Substitution with fluorine is preferable because it improves compatibility with a compound in which fluorine is substituted.

The tertiary amine among the above-described aryl amines is preferably an organic compound represented by General Formula (Gam1) below, for example.

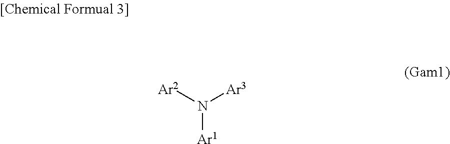

Note that in General Formula (Gam1) above, Ar¹ to Ar³ represent substituted or unsubstituted aryl groups each having 6 to 14 carbon atoms, and the aryl groups may be bonded to each other to form a ring. In the case where any of Ar¹ to Ar³ has a substituent, the substituent may be a group formed by connecting a plurality of diaryl amino groups or a plurality of carbazolyl groups. An ether bond, a sulfide bond, or a bond via an amine may be included; any of these bonds preferably exists between a plurality of aryl groups, in which case the solubility in an organic solvent is improved. Also when an alkyl group is included as a substituent, the alkyl group may be bonded through an ether bond, a sulfide bond, or a bond via an amine.

Specific examples of the tertiary amine represented by General Formula (Gam1) above include organic compounds represented by Structural Formula (Am1-1) to Structural Formula (Am1-8) below. Note that another material having a hole-transport property may be appropriately mixed with any of these tertiary amines.

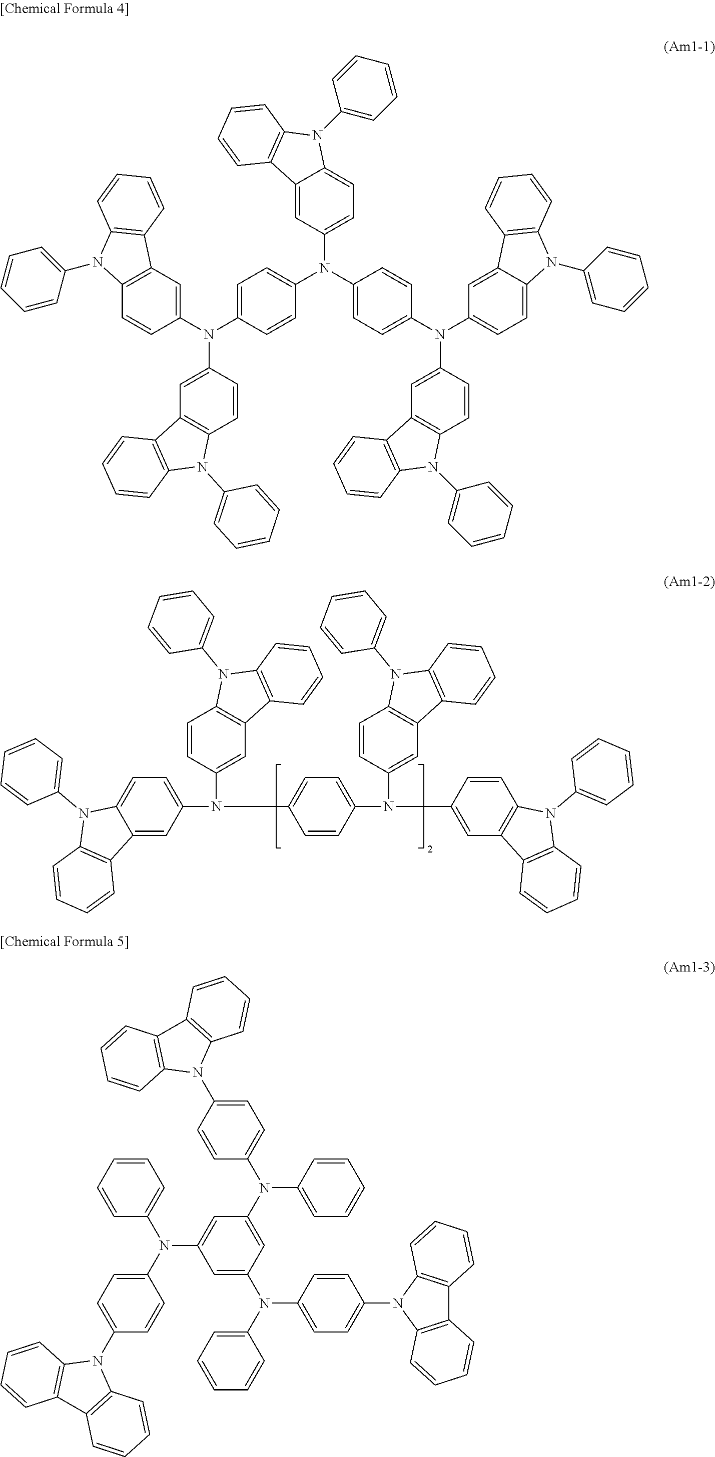

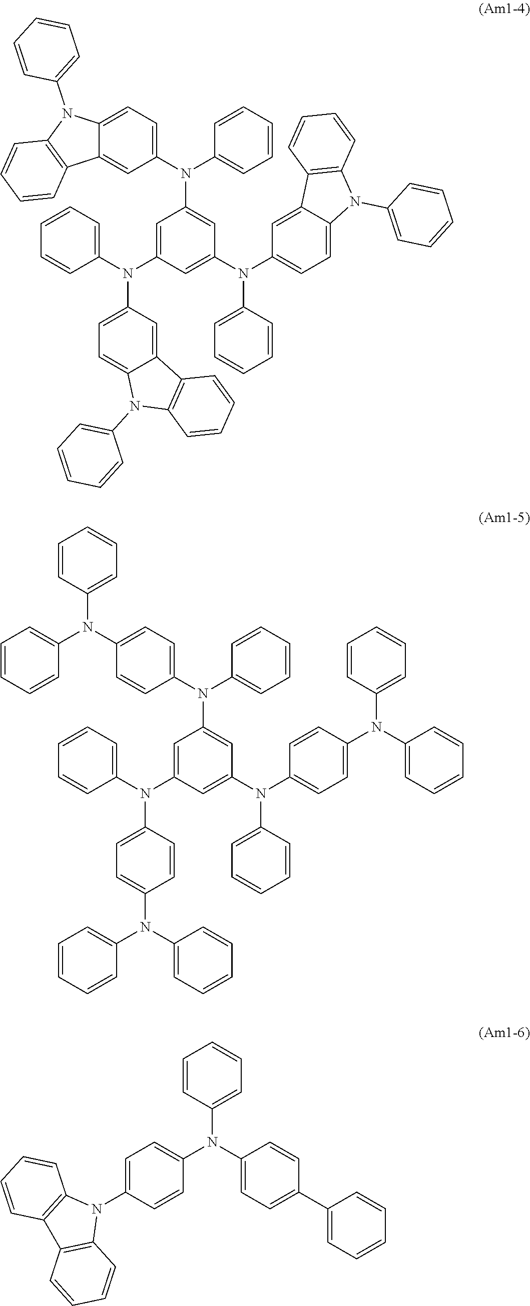

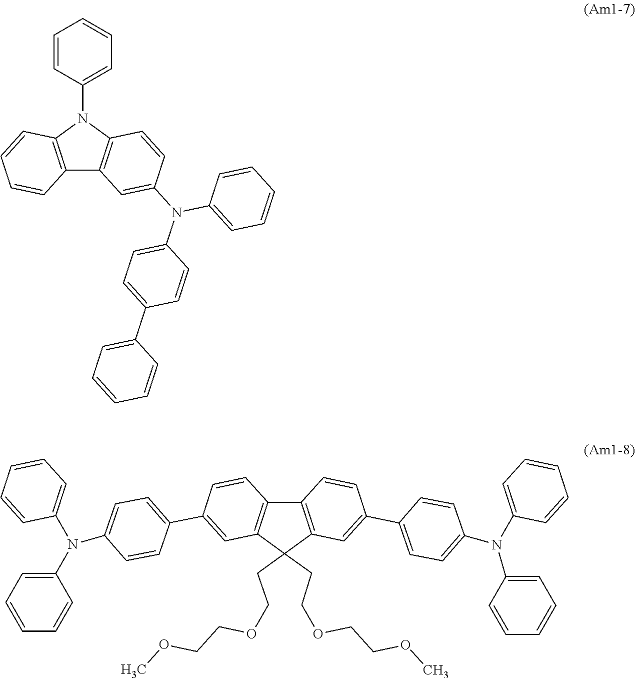

The amine used as the above monomer may be a secondary amine.

The secondary amine is preferably an organic compound represented by General Formula (Gam2) below, for example.

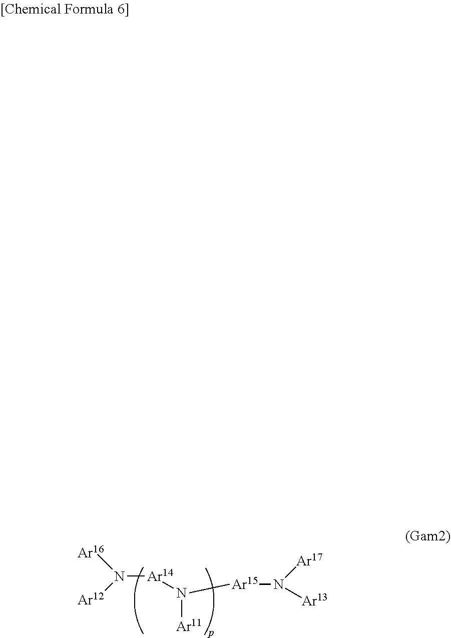

Note that in General Formula (Gam2) above, one or more of Ar¹¹ to Ar¹³ represent hydrogen, the others thereof represent substituted or unsubstituted aromatic rings each having 6 to 14 carbon atoms, and Ar¹⁴ to Ar¹⁷ represent substituted or unsubstituted aromatic rings each having 6 to 14 carbon atoms. Note that Ar¹² and Ar¹⁶ may be bonded to each other to form a ring, Ar¹⁴ and Ar¹⁶ may be bonded to each other to form a ring, Ar¹¹ and Ar¹⁴ may be bonded to each other to form a ring, Ar¹⁴ and Ar¹⁵ may be bonded to each other to form a ring, Ar¹⁵ and Ar¹⁷ may be bonded to each other to form a ring, and Ar¹³ and Ar¹⁷ may be bonded to each other to form a ring. Furthermore, p represents an integer of 0 to 1000, and preferably represents 0 to 3. Note that the molecular weight of the organic compound represented by General Formula (Gam2) is preferably less than or equal to 100000. As the aromatic ring having 6 to 14 carbon atoms, a benzene ring, a bisbenzene ring, a naphthalene ring, a fluorene ring, a phenanthrene ring, an anthracene ring, or the like can be used.

Specific examples of the secondary amine (having an NH group) represented by General Formula (Gam2) above include organic compounds represented by Structural Formula (Am2-1) to Structural Formula (Am2-32) below. To improve the compatibility, it is preferable that the secondary amine be used as the above monomer, a compound mixed in addition to the heteropoly acid be a fluoride, any of fluorides represented by Structural Formula (Am2-22) to Structural Formula (Am2-28), Structural Formula (Am2-31), and the like be used as the secondary amine, and the fluoride be contained in varnish.

The varnish described in this specification and the like can be rephrased as ink. The ink described in this specification and the like can be rephrased as varnish.

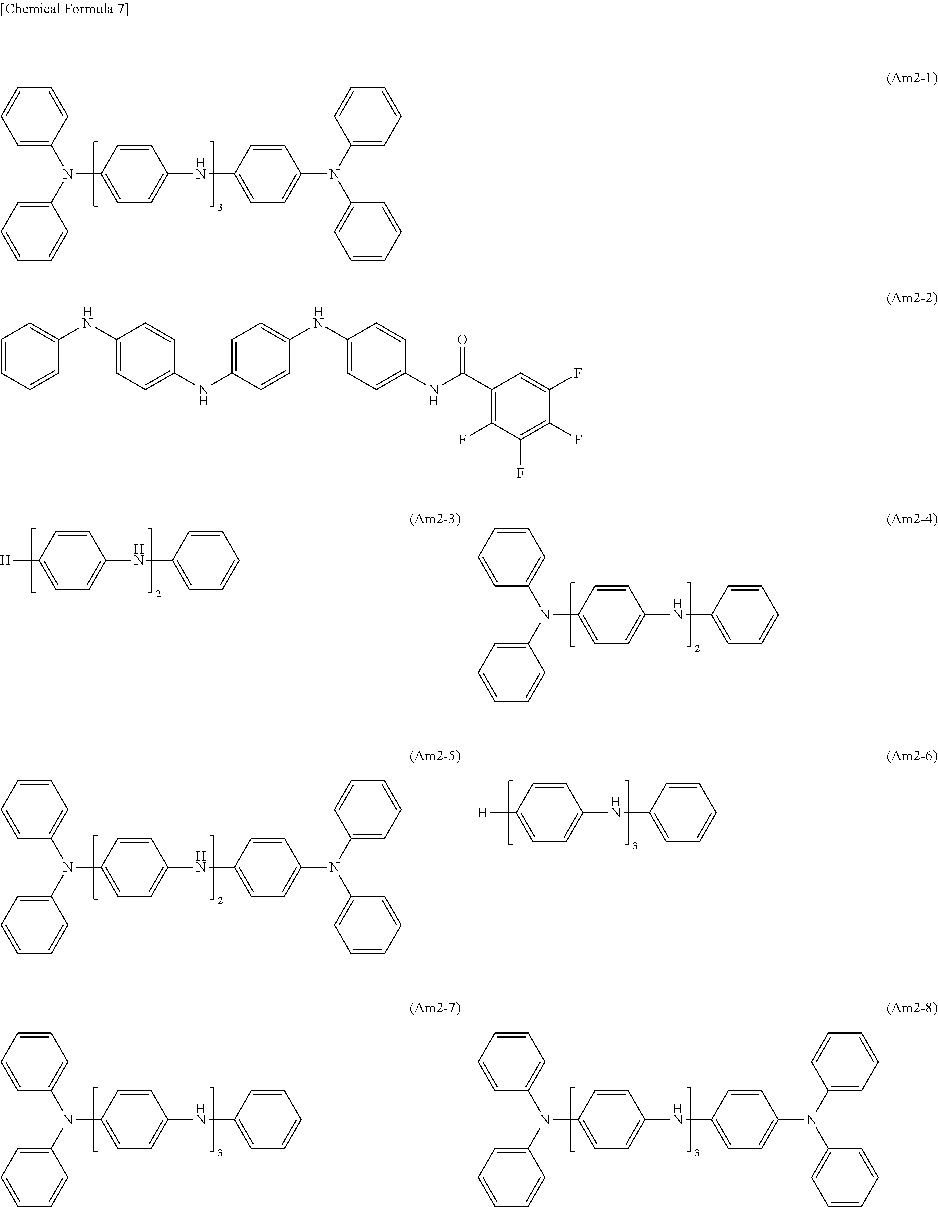

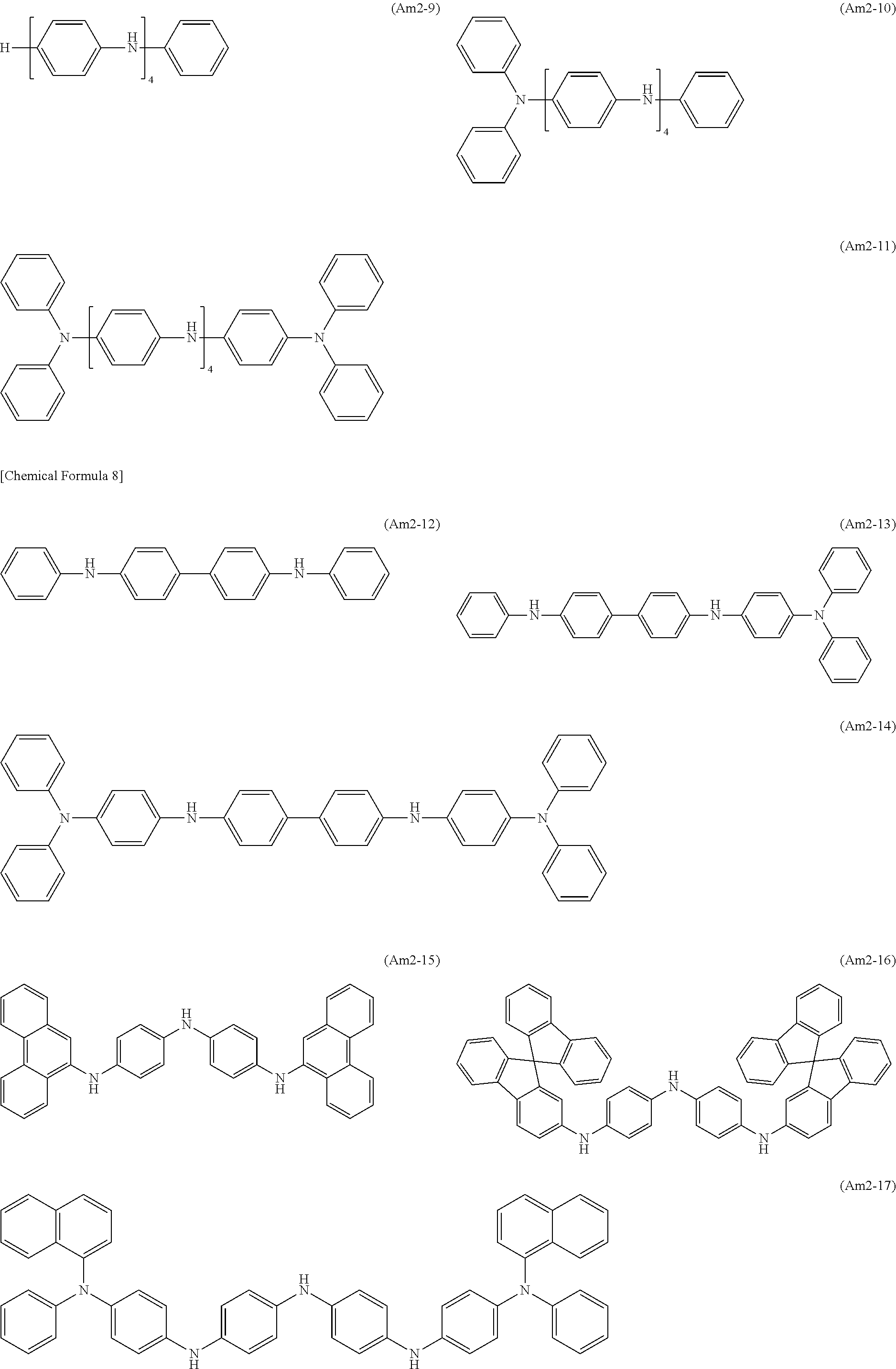

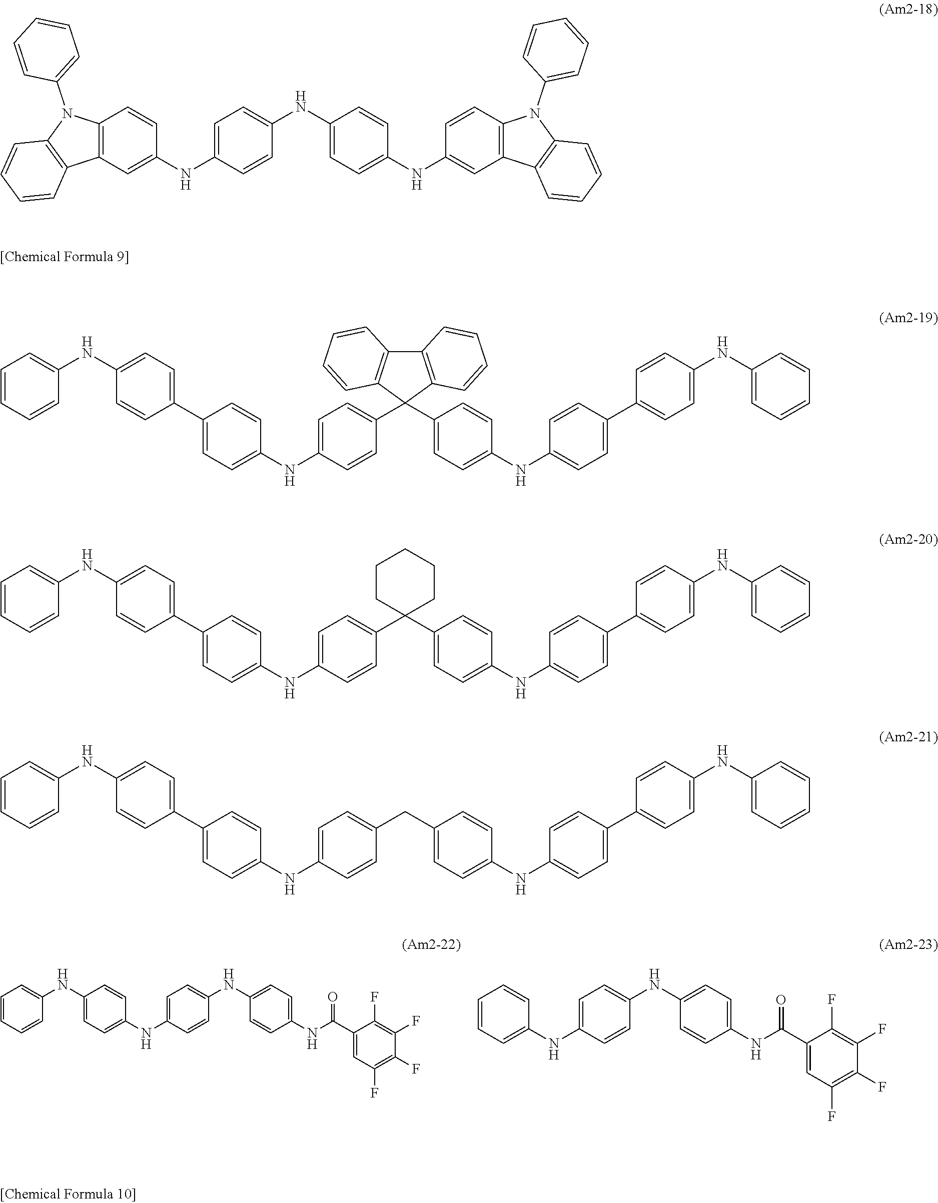

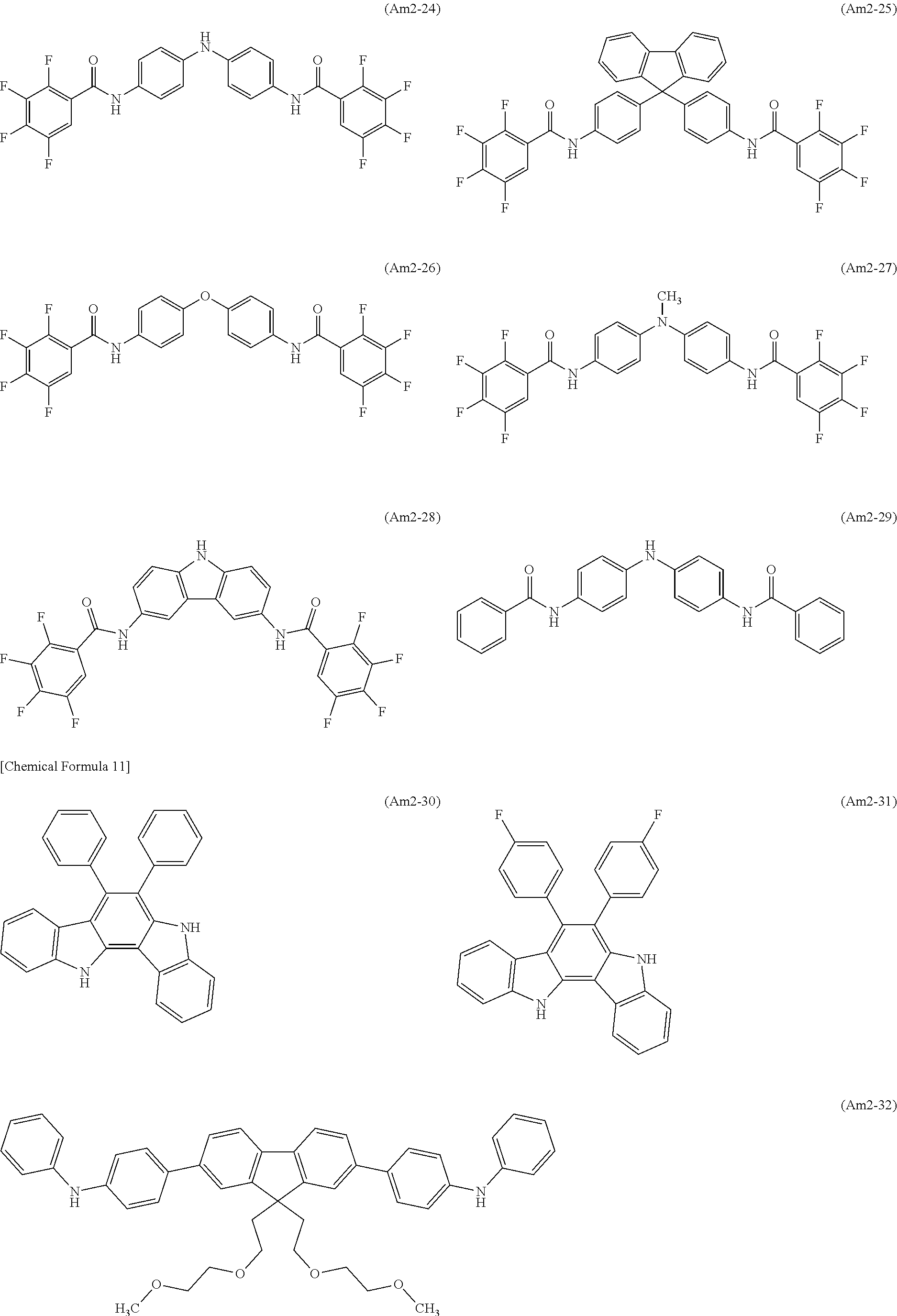

As the material with a hole-transport property, a thiophene derivative may be used instead of an aryl amine. Specific and preferred examples of the thiophene derivative include organic compounds represented by Structural Formula (T-1) to Structural Formula (T-4) below, a polythiophene, and poly(3,4-ethylenedioxythiophene) (PEDOT). A thiophene derivative facilitates generation of carriers and improves conductivity by being mixed with a heteropoly acid.

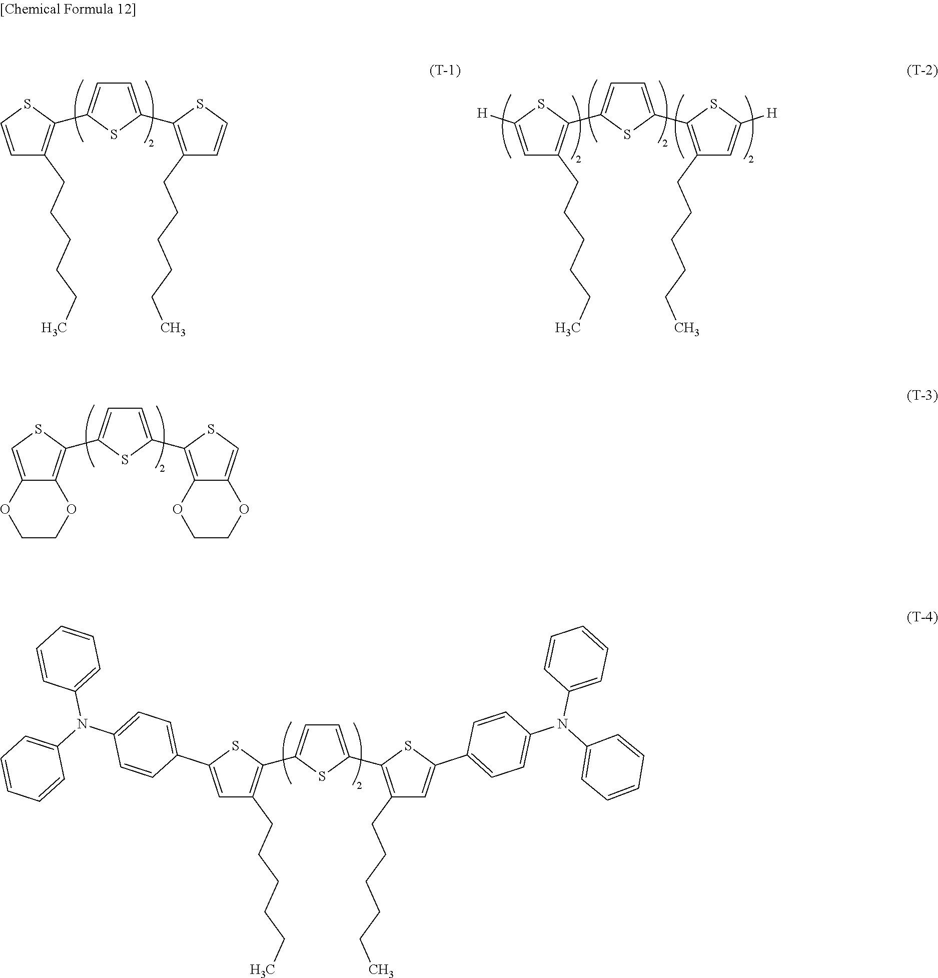

In some cases, it is preferable that a material having an acceptor property other than a heteropoly acid be contained in addition to the above material having a hole-transport property and the above heteropoly acid. Examples of the material having an acceptor property include a sulfonic acid compound, a fluorine compound, a trifluoroacetic acid compound, a propionic acid compound, and a metal oxide. A preferred sulfonic acid compound is an arylsulfonic acid compound.

It is only required that the arylsulfonic acid has a sulfo group; a sulfonic acid, a sulfonate, an alkoxysulfonic acid, a halogenated sulfonic acid, or a sulfonic acid anion can be used. Specifically, any of the above-described groups can be used as the sulfo group. Two or more of these sulfo groups may be contained. As the aryl group of the arylsulfonic acid, a substituted or unsubstituted aryl group having 6 to 16 carbon atoms can be used. As the aryl group, for example, a phenyl group, a biphenyl group, a naphthyl group, a fluorenyl group, a phenanthrenyl group, an anthryl group, a pyrenyl group, or the like can be used, and a naphthyl group is preferable because it has favorable solubility in an organic solvent and a favorable transport property. These arylsulfonic acids may each have a plurality of aryl groups; an aryl group in which fluorine is substituted is preferably contained, in which case the LUMO level can be adjusted to be deep (high in the negative direction). An ether bond, a sulfide bond, or a bond via an amine may be included; any of these bonds preferably exists between a plurality of aryl groups, in which case the solubility in an organic solvent is improved. Also when an alkyl group is included as a substituent, the alkyl group may be bonded through an ether bond, a sulfide bond, or a bond via an amine. A plurality of the arylsulfonic acids may be substituted in a polymer. As the polymer, polyethylene, nylon, polystyrene, polyfluorenylene, or the like can be used; polystyrene or polyfluorenylene is preferred because of its favorable conductivity.

Specific and preferred examples of the arylsulfonic acid compound include organic compounds represented by Structural Formula (S-1) to Structural Formula (S-15) below. A polymer having a sulfo group such as poly(4-styrenesulfonic acid) (PSS) can also be used. Use of the arylsulfonic acid compound enables accepting electrons from an electron donor with a shallow HOMO level (e.g., an amine compound, a carbazole compound, or a thiophene compound), and mixing with an electron donor enables a property of injecting or transporting holes from an electrode. When a fluorine compound is employed, the LUMO level can be adjusted to be deeper (the energy level can be higher in the negative direction).

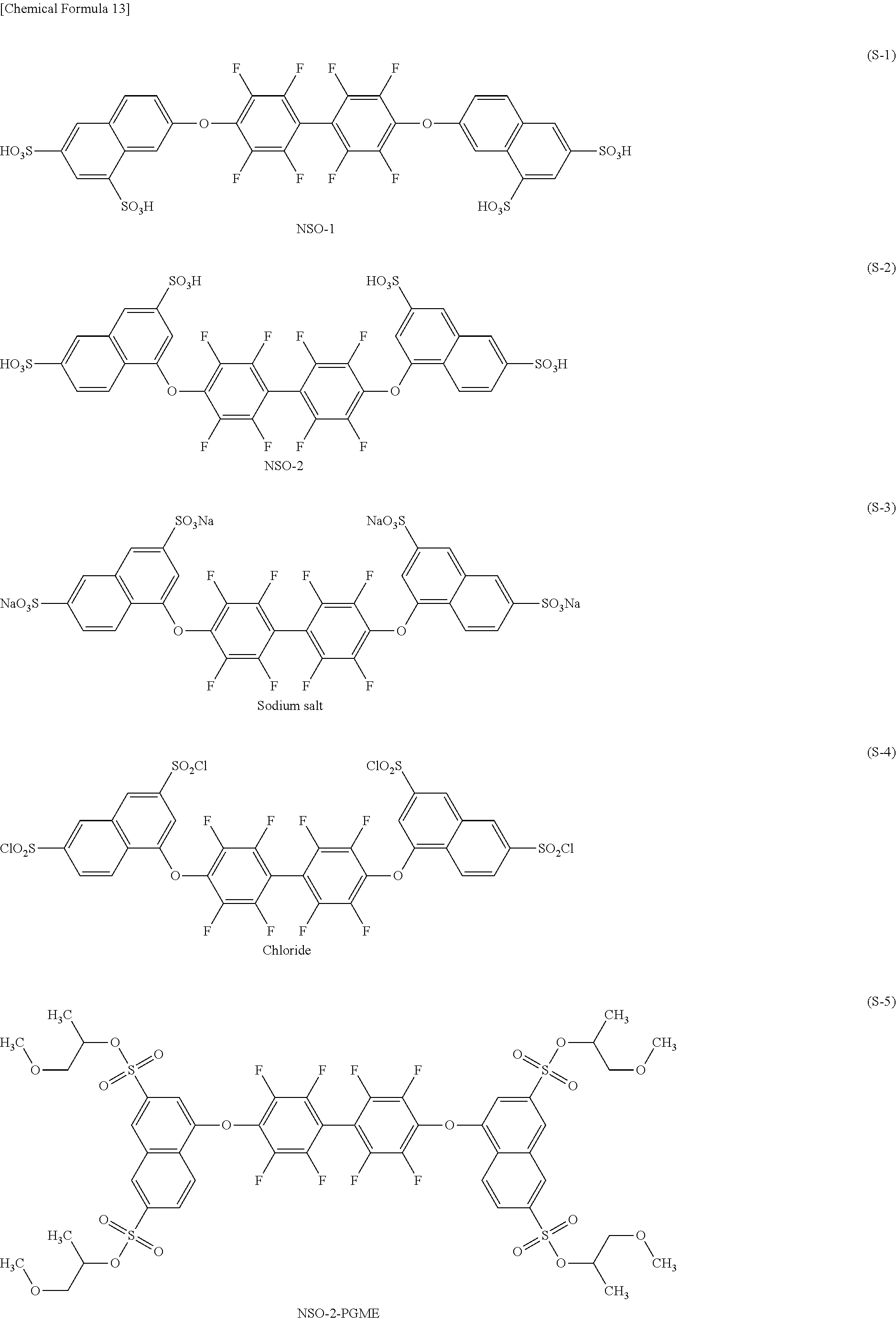

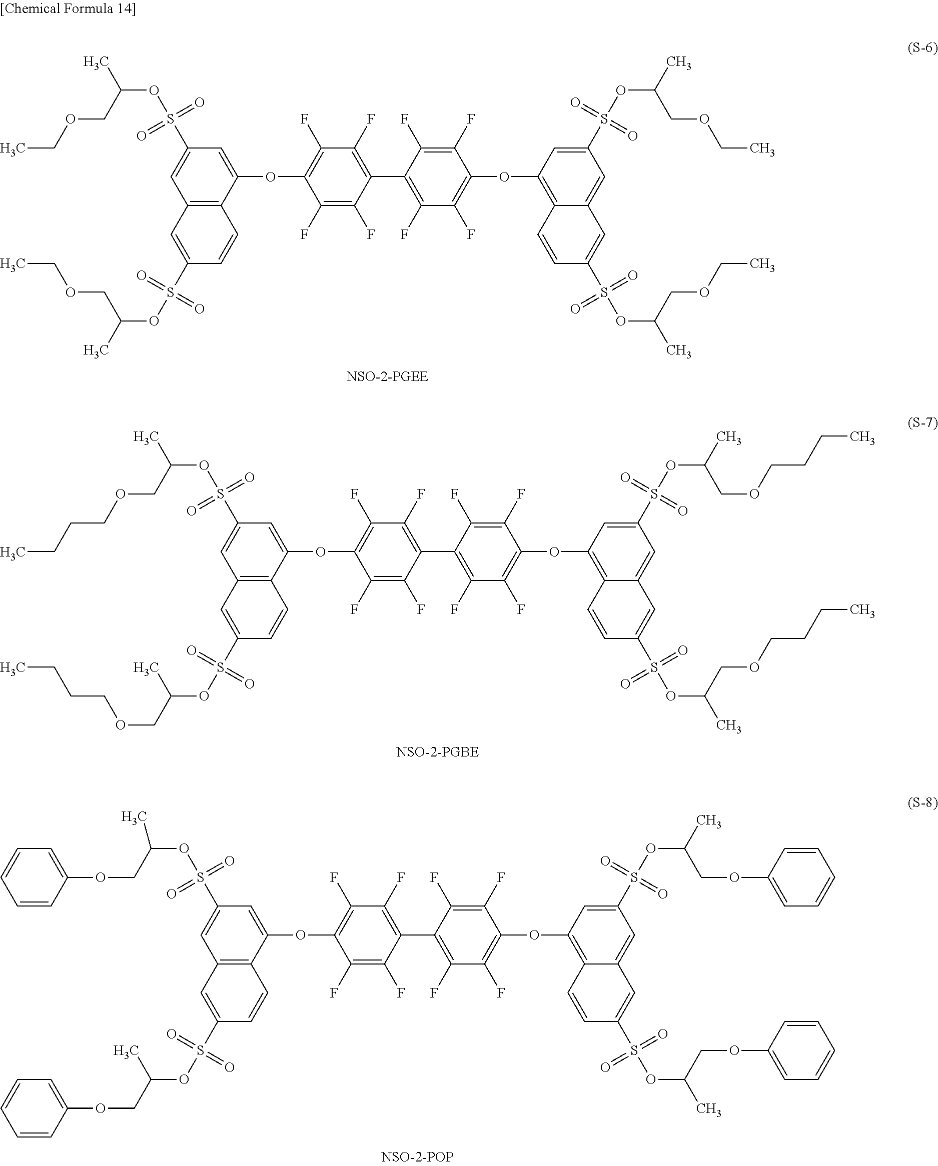

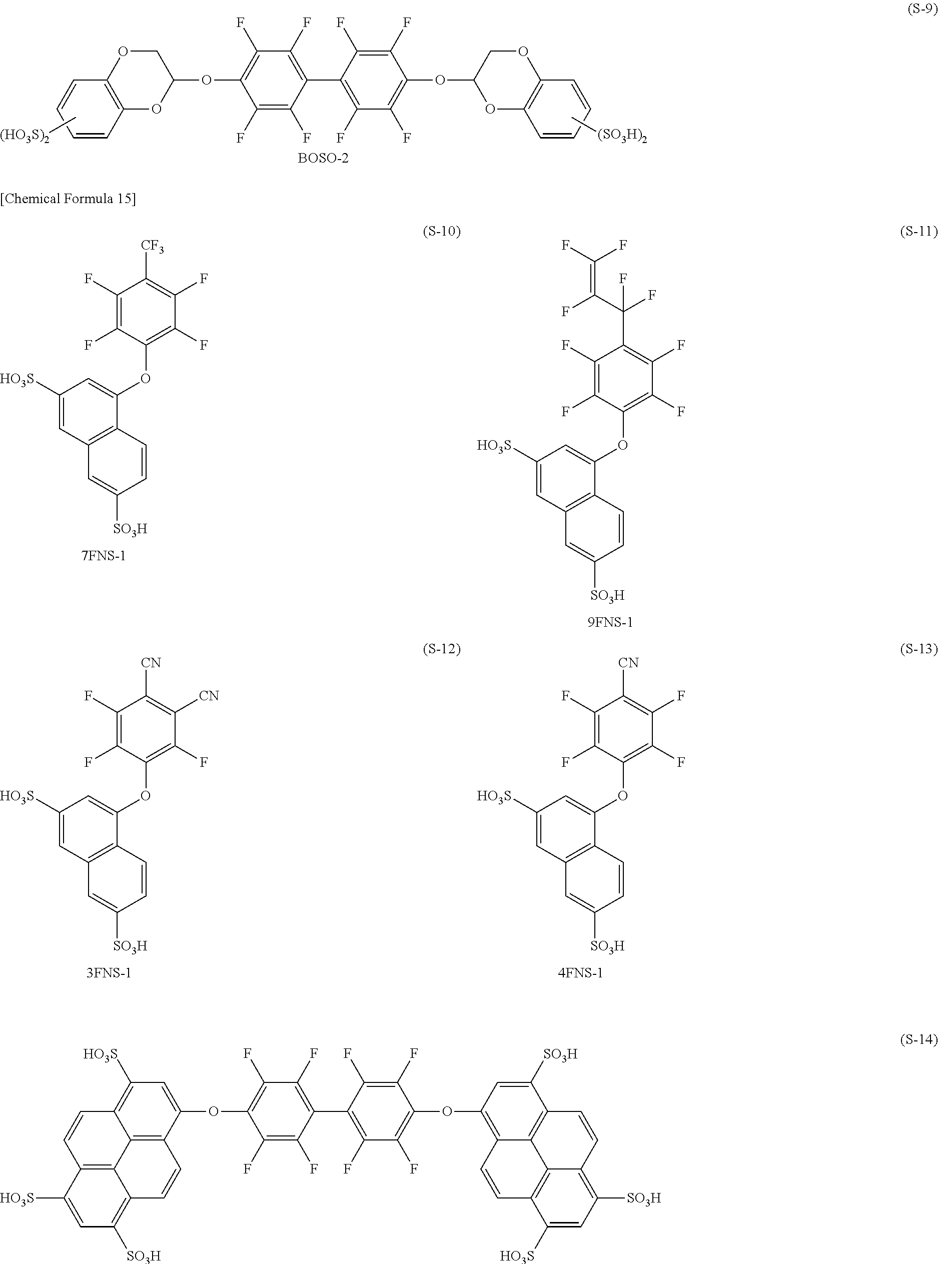

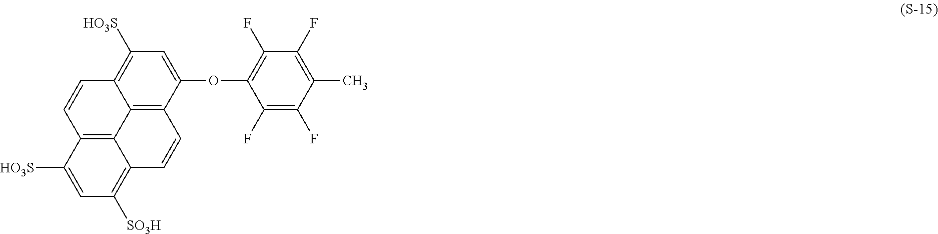 [SMILES: Cc1c(F)c(F)c(Oc2cc(S(=O)(=O)O)c3ccc4c(S(=O)(=O)O)cc(S(=O)(=O)O)c5ccc2c3c54)c(F)c1F]

Other than the arylsulfonic acid compound, a cyano compound such as a tetracyanoquinodimethane compound can be used as an electron acceptor. Specifically, 2,3,5,6-tetrafluoro-7,7,8,8-tetracyano-quinodimethane (F4TCNQ), dipyrazino[2,3-f:2′,3′-h]quinoxaline-2,3,6,7,10,11-hexacarbonitrile (HAT-CN6), and the like can be given as examples.

Note that ink in which the above monomer is mixed preferably contains one or both of a 3,3,3-trifluoropropyltrimethoxysilane compound and a phenyltrimethoxysilane compound, in which case wettability at the time of film formation by a wet process is improved.

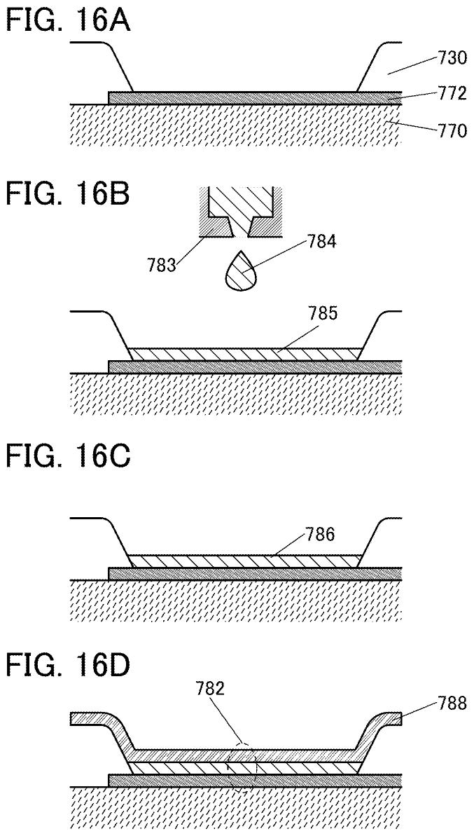

Here, a method for forming a layer 786 containing a light-emitting substance by a droplet discharge method will be described with reference to FIG. 16. FIG. 16A to FIG. 16D are cross-sectional views illustrating a method for forming the layer 786 containing a light-emitting substance.

First, a conductive film 772 is formed over a planarization insulating film 770, and an insulating film 730 is formed to cover part of the conductive film 772 (see FIG. 16A).

Then, a droplet 784 is discharged from a droplet discharge apparatus 783 to the conductive film 772 exposed in an opening of the insulating film 730, so that a layer 785 containing a composition is formed. The droplet 784 is a composition containing a solvent and is attached onto the conductive film 772 (see FIG. 16B).

Note that the step of discharging the droplet 784 may be performed under reduced pressure.

Next, the solvent is removed from the layer 785 containing a composition, and the layer is solidified to form the layer 786 containing a light-emitting substance (see FIG. 16C).

As the solvent removing method, a drying process or a heating process may be performed.

Next, a conductive film 788 is formed over the layer 786 containing a light-emitting substance; thus, a light-emitting element 782 is formed (see FIG. 16D).

When the layer 786 containing a light-emitting substance is formed by a droplet discharge method in this manner, the composition can be selectively discharged; accordingly, waste of the material can be reduced. Furthermore, a lithography process or the like for shaping is not needed, and thus, the process can be simplified and cost reduction can be achieved.

The droplet discharge method mentioned above is a general term for a method with a droplet discharge means such as a nozzle having a composition discharge outlet or a head having one or a plurality of nozzles.

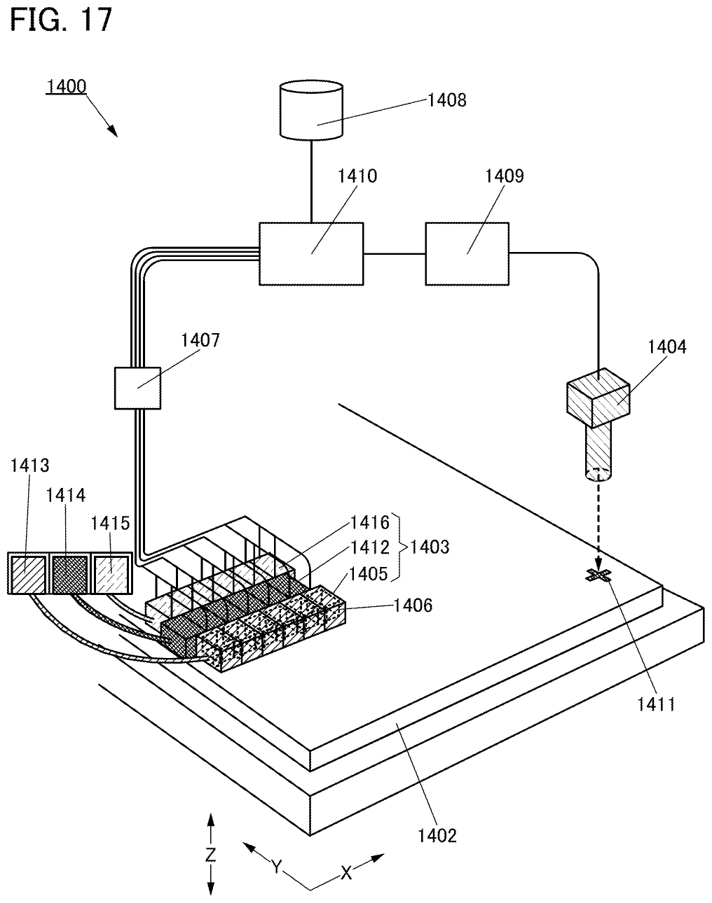

Next, a droplet discharge apparatus used for the droplet discharge method will be described with reference to FIG. 17. FIG. 17 is a conceptual diagram illustrating a droplet discharge apparatus 1400.

The droplet discharge apparatus 1400 includes a droplet discharge means 1403. The droplet discharge means 1403 further includes a head 1405, a head 1412, and a head 1416.

The head 1405, the head 1412, and the head 1416 are connected to a control means 1407 that is controlled by a computer 1410; thus, a preprogrammed pattern can be drawn.

The drawing may be conducted at a timing, for example, based on a marker 1411 formed over a substrate 1402. Alternatively, the reference point may be determined on the basis of an outer edge of the substrate 1402. Here, the marker 1411 is detected by an imaging means 1404 and converted into a digital signal by an image processing means 1409. The computer 1410 recognizes the digital signal, generates a control signal, and transmits the control signal to the control means 1407.

An image sensor or the like utilizing a charge coupled device (CCD) or a complementary metal-oxide-semiconductor (CMOS) can be used as the imaging means 1404. Note that information on a pattern to be formed on the substrate 1402 is stored in a storage medium 1408, and a control signal is transmitted to the control means 1407 on the basis of the information, so that the head 1405, the head 1412, and the head 1416 of the droplet discharge means 1403 can be individually controlled. Materials to be discharged are supplied to the head 1405, the head 1412, and the head 1416 from a material supply source 1413, a material supply source 1414, and a material supply source 1415, respectively, through pipes.

Inside the head 1405, the head 1412, and the head 1416, a space indicated by a dotted line 1406 to be filled with a liquid material and a nozzle serving as a discharge outlet are provided. Although not illustrated, the inside structure of the head 1412 is similar to that of the head 1405. When the nozzle sizes of the head 1405 and the head 1412 are different from each other, different materials with different widths can be discharged simultaneously. Each head can discharge a plurality of kinds of light-emitting materials or the like to draw a pattern. In the case of drawing a pattern over a large area, the same material can be simultaneously discharged from a plurality of nozzles in order to improve throughput. When a large substrate is used, the head 1405, the head 1412, and the head 1416 can freely scan the substrate in the directions of arrows X, Y, and Z in FIG. 17, a region in which a pattern is drawn can be freely set, and the same patterns can be drawn on one substrate.

Furthermore, the step of discharging the composition may be performed under reduced pressure. The substrate may be heated at the time of discharging. The discharge of the composition is followed by one or both of a drying step and a baking step. Both the drying and baking steps are heat treatments but different in purpose, temperature, and time. The drying step and the baking step are performed by laser light irradiation, rapid thermal annealing, heating in a heating furnace, or the like under normal pressure or reduced pressure in the air or an inert atmosphere such as nitrogen. Note that there is no particular limitation on the timing of the heat treatment and the number of times of the heat treatment. The temperature for adequately performing the drying and baking steps depends on the material of the substrate and the properties of the composition.

In the above-described manner, the layer 786 containing a light-emitting substance can be formed with the droplet discharge apparatus.

When the layer 786 containing a light-emitting substance is formed with the droplet discharge apparatus by a wet process with a composition in which any of a variety of organic materials and organic-inorganic halide perovskites is dissolved or dispersed in a solvent, any of various organic solvents can be used to form a composition for application. As examples of a variety of organic solvents that can be used for the composition, benzene, toluene, xylene, mesitylene, tetrahydrofuran, dioxane, ethanol, methanol, n-propanol, isopropanol, n-butanol, t-butanol, acetonitrile, dimethylsulfoxide, dimethylformamide, chloroform, methylene chloride, carbon tetrachloride, ethyl acetate, hexane, cyclohexane, and the like are given. In particular, a low-polarity benzene derivative such as benzene, toluene, xylene, or mesitylene is preferably used, in which case a solution with a suitable concentration can be obtained and a material contained in a droplet can be prevented from deteriorating due to oxidation or the like. Furthermore, in light of the uniformity of a formed film or the uniformity of film thickness, the boiling point is preferably 100° C. or higher, and toluene, xylene, or mesitylene is further preferable.

Note that the above-described structure can be combined with another embodiment and another structure in this embodiment as appropriate.

The organic compound with an electron-transport property contained in the electron-transport region 121 of the light-emitting device of one embodiment of the present invention preferably has an ordinary refractive index of higher than or equal to 1.50 and lower than or equal to 1.75 for light with a wavelength (λ_{B}) in the range of 455 nm to 465 nm or an ordinary refractive index of higher than or equal to 1.45 and lower than or equal to 1.70 for light with a wavelength of 633 nm.

Note that a refractive index in this specification, including that of the above organic compound with an electron-transport property, is determined by performing measurement on a thin film of the material; in the case where the material in the form of such a thin film has anisotropy, the refractive index for an ordinary ray sometimes differs from that for an extraordinary ray. When a thin film subjected to the measurement is in such a state, anisotropy analysis can be performed to separately calculate the ordinary refractive index and the extraordinary refractive index. In this specification, when the material subjected to the measurement has both the ordinary refractive index and the extraordinary refractive index, the ordinary refractive index is used as an indicator.

Since the electron-transport region 121 includes such a material, a low-refractive-index layer can be provided therein. Providing a low-refractive-index layer in the EL layer allows the light-emitting device to have improved outcoupling efficiency and high emission efficiency. In general, the refractive index of an organic compound used in a light-emitting device is approximately 1.8 to 1.9. The light-emitting device of one embodiment of the present invention is provided with the electron-transport region 121 including a low-refractive-index layer and can accordingly have favorable emission efficiency.

In the case where the light-emitting device of one embodiment of the present invention is a blue-light-emitting device, the electron-transport region 121 preferably includes a layer whose ordinary refractive index for light with λ_{B} is higher than or equal to 1.50 and lower than 1.75, further preferably includes a layer whose ordinary refractive index for light with λ_{B} is higher than or equal to 1.50 and lower than 1.70. The organic compound with an electron-transport property contained in the electron-transport region preferably has an ordinary refractive index of higher than or equal to 1.50 and lower than or equal to 1.75, further preferably higher than or equal to 1.50 and lower than or equal to 1.70, for light with λ_{B}.

In principle, the refractive index is higher on a shorter wavelength side and is lower on a longer wavelength side; thus, the organic compound with an electron-transport property used in the electron-transport layer 114 of one embodiment of the present invention preferably has an ordinary refractive index of higher than or equal to 1.45 and lower than or equal to 1.70 for light with a wavelength of 633 nm.

Note that the organic compound with an electron-transport property preferably includes an alkyl group or a cycloalkyl group. When these have an alkyl group or a cycloalkyl group, the refractive index can be reduced to allow the electron-transport layer 114 to have a low refractive index.

The alkyl group of the organic compound with an electron-transport property is preferably a branched alkyl group, particularly preferably an alkyl group having 3 or 4 carbon atoms, and particularly preferably a tert-butyl group.

The organic compound with an electron-transport property preferably includes at least one six-membered heteroaromatic ring having 1 to 3 nitrogen atoms, a plurality of aromatic hydrocarbon rings each of which has 6 to 14 carbon atoms in the ring and at least two of which are benzene rings, and an organic compound having a plurality of hydrocarbon groups forming a bond by sp³ hybrid orbitals.

In such an organic compound, the total number of carbon atoms forming a bond by sp³ hybrid orbitals preferably accounts for higher than or equal to 10% and lower than or equal to 60%, further preferably higher than or equal to 10% and lower than or equal to 50% of the total number of carbon atoms in the molecule of the organic compound. In other words, the proportion of the number of carbon atoms forming a bond by sp³ hybrid orbitals is preferably higher than or equal to 10% and lower than or equal to 60%, further preferably higher than or equal to 10% and lower than or equal to 50% of the total number of carbon atoms in the molecule of the organic compound. Alternatively, when the above organic compound is subjected to ¹H-NMR measurement, the integral value of signals at lower than 4 ppm is preferably ½ or more of the integral value of signals at 4 ppm or higher.

It is preferable that all the hydrocarbon groups forming a bond by sp³ hybrid orbitals in the organic compound be bonded to the above aromatic hydrocarbon rings each having 6 to 14 carbon atoms in the ring, and the LUMO of the organic compound not be distributed over the aromatic hydrocarbon rings.

The organic compound with an electron-transport property is preferably contained in the electron-transport layer 114 in the electron-transport region 121.

The organic compound with an electron-transport property is preferably an organic compound represented by General Formula (Gₑ₁1) below.

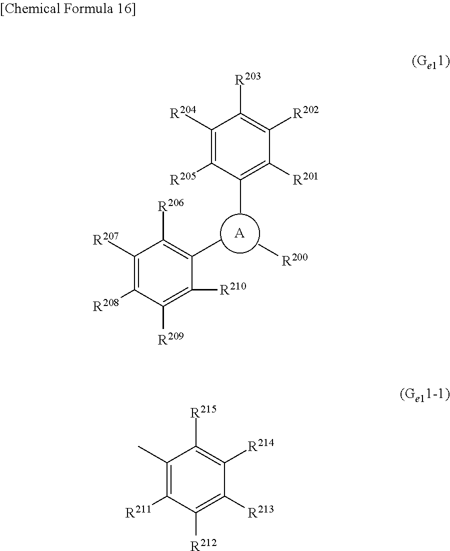

In the formula, A represents a six-membered heteroaromatic ring having 1 to 3 nitrogen atoms, and is preferably any of a pyridine ring, a pyrimidine ring, a pyrazine ring, a pyridazine ring, and a triazine ring.

Furthermore, R²⁰⁰ represents any of hydrogen, an alkyl group having 1 to 6 carbon atoms, an alicyclic group having 3 to 10 carbon atoms, and a substituent represented by General Formula (Gₑ₁1-1) above.

At least one of R²⁰¹ to R²¹⁵ represents a phenyl group having a substituent and the others each independently represent any of hydrogen, an alkyl group having 1 to 6 carbon atoms, an alicyclic group having 3 to 10 carbon atoms, a substituted or unsubstituted aromatic hydrocarbon group having 6 to 14 carbon atoms in the ring, and a substituted or unsubstituted pyridyl group. Note that R²⁰¹, R²⁰³, R²⁰⁵, R²⁰⁶, R²⁰⁸, R²¹⁰, R²¹¹, R²¹³, and R²¹⁵ are preferably hydrogen. The phenyl group having a substituent has one or two substituents, which each independently represent any of an alkyl group having 1 to 6 carbon atoms, an alicyclic group having 3 to 10 carbon atoms, and a substituted or unsubstituted aromatic hydrocarbon group having 6 to 14 carbon atoms in the ring.

The organic compound represented by General Formula (Gₑ₁1) above has a plurality of hydrocarbon groups selected from an alkyl group having 1 to 6 carbon atoms and an alicyclic group having 3 to 10 carbon atoms, and the proportion of the total number of carbon atoms forming a bond by sp³ hybrid orbitals is higher than or equal to 10% and lower than or equal to 60% of the total number of carbon atoms in the molecule of the organic compound.

The organic compound with an electron-transport property is preferably an organic compound represented by General Formula (Gₑ₁2) below.

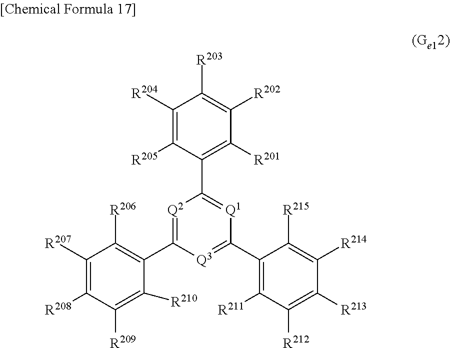

In the formula, two or three of Q¹ to Q³ represent N; in the case where two of the Q¹ to Q³ are N, the rest represents CH.

At least one of R²⁰¹ to R²¹⁵ represents a phenyl group having a substituent and the others each independently represent any of hydrogen, an alkyl group having 1 to 6 carbon atoms, an alicyclic group having 3 to 10 carbon atoms, a substituted or unsubstituted aromatic hydrocarbon group having 6 to 14 carbon atoms in the ring, and a substituted or unsubstituted pyridyl group. Note that R²⁰¹, R²⁰³, R²⁰⁵, R²⁰⁶, R²⁰⁸, R²¹⁰, R²¹¹, R²¹³, and R²¹⁵ are preferably hydrogen. The phenyl group having a substituent has one or two substituents, which each independently represent any of an alkyl group having 1 to 6 carbon atoms, an alicyclic group having 3 to 10 carbon atoms, and a substituted or unsubstituted aromatic hydrocarbon group having 6 to 14 carbon atoms in the ring.

It is preferable that the organic compound represented by General Formula (Gₑ₁2) above have a plurality of hydrocarbon groups selected from an alkyl group having 1 to 6 carbon atoms and an alicyclic group having 3 to 10 carbon atoms, and the proportion of the number of carbon atoms forming a bond by sp³ hybrid orbitals be higher than or equal to 10% and lower than or equal to 60% of the total number of carbon atoms in the molecule of the organic compound.

In the organic compound represented by General Formula (Gₑ₁1) or (Gₑ₁2) above, the phenyl group having a substituent is preferably a group represented by General Formula (Gₑ₁1-2) below.

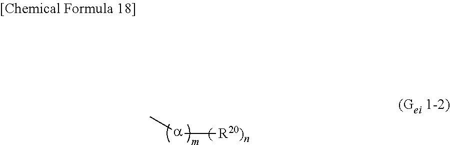

In the formula, α represents a substituted or unsubstituted phenylene group and is preferably a meta-substituted phenylene group. In the case where the meta-substituted phenylene group has a substituent, the substituent is also preferably meta-substituted. The substituent is preferably an alkyl group having 1 to 6 carbon atoms or an alicyclic group having 3 to 10 carbon atoms, further preferably an alkyl group having 1 to 6 carbon atoms, and still further preferably a tert-butyl group.

R²⁰ represents an alkyl group having 1 to 6 carbon atoms, an alicyclic group having 3 to 10 carbon atoms, or a substituted or unsubstituted aromatic hydrocarbon group having 6 to 14 carbon atoms in the ring.

Furthermore, m and n represent 1 or 2. In the case where m is 2, a plurality of α's may be the same or different from each other. In the case where n is 2, a plurality of R²⁰s may be the same or different from each other. R²⁰ is preferably a phenyl group, and it is further preferable that the phenyl group of R²⁰ have an alkyl group having 1 to 6 carbon atoms or an alicyclic group having 3 to 10 carbon atoms at one or both of the two meta-positons. The substituent at one or both of the two meta-positons of the phenyl group is preferably an alkyl group having 1 to 6 carbon atoms, further preferably a tert-butyl group.

In the light-emitting device of one embodiment of the present invention, the electron-transport layer 114 in the electron-transport region 121 preferably contains a metal complex of an alkali metal or an alkaline earth metal together with the organic compound having an electron-transport property. The metal complex of an alkali metal or an alkaline earth metal is preferably a metal complex of lithium. The metal complex of an alkali metal or an alkaline earth metal preferably has a ligand including an 8-quinolinolato structure. The metal complex of an alkali metal or an alkaline earth metal is preferably 8-quinolinolato-lithium or the like, for example.

The ligand including an 8-quinolinolato structure preferably has an alkyl group. In the case where a lithium complex having a ligand including an 8-quinolinolato has an alkyl group, the number of alkyl groups of the complex is preferably one. The alkyl group of the metal complex of an alkali metal preferably has one, two, or three carbon atoms, and is particularly preferably a methyl group. It is possible that 8-quinolinolato-lithium having an alkyl group be a metal complex with a low refractive index. Specifically, the ordinary refractive index of the metal complex in a thin film state for light with a wavelength in the range of 455 nm to 465 nm can be higher than or equal to 1.45 and lower than or equal to 1.70, and the ordinary refractive index thereof for light with a wavelength of 633 nm can be higher than or equal to 1.40 and lower than or equal to 1.65.

In particular, the use of 6-alkyl-8-quinolinolato-lithium having an alkyl group at the 6 position has an effect of lowering the driving voltage of a light-emitting device. Of 6-alkyl-8-quinolinolato-lithium, 6-methyl-8-quinolinolato-lithium is preferably used.

Here, the above-described 6-alkyl-8-quinolinolato-lithium can be represented by General Formula (G_{iq}1) below.

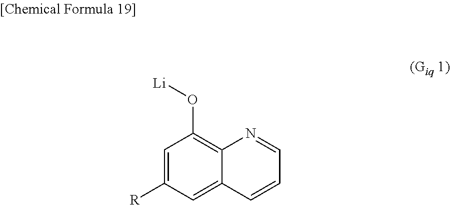

In General Formula (G_{iq}1) above, R represents an alkyl group having 1 to 3 carbon atoms.

Of the metal complexes represented by General Formula (G_{iq}1) above, a metal complex represented by Structural Formula (100) below is a preferred embodiment.

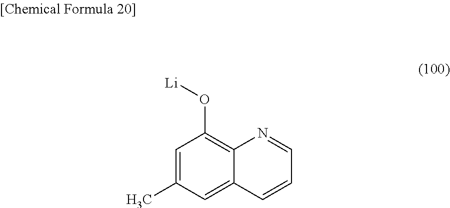

The organic compound with an electron-transport property used in the electron-transport layer 114 of the light-emitting device of one embodiment of the present invention preferably has an alkyl group having 3 or 4 carbon atoms as described above; in particular, the organic compound with an electron-transport property preferably has a plurality of such alkyl groups. However, too many alkyl groups in the molecule reduce the carrier-transport property; thus, the proportion of carbon atoms forming a bond by sp³ hybrid orbitals in the organic compound with an electron-transport property is preferably higher than or equal to 10% and lower than or equal to 60%, further preferably higher than or equal to 10% and lower than or equal to 50% of the total number of carbon atoms in the organic compound. The organic compound with an electron-transport property having such a structure can achieve a low refractive index without a significant impairment of the electron-transport property.

When such an organic compound is subjected to measurement by ¹H-NMR (proton nuclear magnetic resonance), the integral value of signals at lower than 4 ppm exceeds the integral value of signals at 4 ppm or higher.

In general, the existence of an alkyl group or a cycloalkyl group is thought to hinder the interaction (also referred to as docking) between the organic compound with an electron-transport property and the metal complex of an alkali metal and cause an increase in driving voltage; however, in the light-emitting device of one embodiment of the present invention, a large increase in driving voltage can be a light-emitting device that includes a low-refractive-index layer in the electron-transport region 121 and has good emission efficiency.

Next, examples of other structures and materials of the light-emitting device of one embodiment of the present invention will be described. The light-emitting device of one embodiment of the present invention includes, as described above, the EL layer 103 formed of a plurality of layers between the pair of electrodes, the anode 101 and the cathode 102. The EL layer 103 includes the light-emitting layer 113 containing a light-emitting material and the hole-transport region 120 and the electron-transport region 121 having the aforementioned structures.

The anode 101 is preferably formed using a metal, an alloy, or a conductive compound having a high work function (specifically, 4.0 eV or more), a mixture thereof, or the like. Specifically, for example, indium oxide-tin oxide (ITO: Indium Tin Oxide), indium oxide-tin oxide containing silicon or silicon oxide, indium oxide-zinc oxide, indium oxide containing tungsten oxide and zinc oxide (IWZO), and the like can be given. These conductive metal oxide films are usually formed by a sputtering method but may also be formed by application of a sol-gel method or the like. An example of a formation method is a method in which indium oxide-zinc oxide is formed by a sputtering method using a target in which 1 to 20 wt % zinc oxide is added to indium oxide. Indium oxide containing tungsten oxide and zinc oxide (IWZO) can also be formed by a sputtering method using a target containing 0.5 to 5 wt % tungsten oxide and 0.1 to 1 wt % zinc oxide with respect to indium oxide. In addition, gold (Au), platinum (Pt), nickel (Ni), tungsten (W), chromium (Cr), molybdenum (Mo), iron (Fe), cobalt (Co), copper (Cu), palladium (Pd), a nitride of a metal material (such as titanium nitride), and the like can be given as examples of the material that is used for the anode 101. Graphene can also be used for the material that is used for the anode 101. Note that when a composite material described later is used for a layer that is in contact with the anode 101 in the EL layer 103, an electrode material can be selected regardless of its work function.

When the anode 101 is formed using a material having a visible-light-transmitting property, the light-emitting device can emit light from the anode side as illustrated in FIG. 1C. When the anode 101 is formed on the substrate side, such a light-emitting device can be what is called a bottom-emission light-emitting device.

Although the EL layer 103 preferably has a stacked-layer structure, there is no particular limitation on the stacked-layer structure, and any of various layer structures such as a hole-injection layer, a hole-transport layer, a light-emitting layer, an electron-transport layer, an electron-injection layer, a carrier-blocking layer (a hole-blocking layer, an electron-blocking layer), an exciton-blocking layer, and a charge-generation layer can be employed. Note that one or more of the above layers are not necessarily provided. In this embodiment, description is made on two kinds of structures: the structure as shown in FIG. 1A which includes, in addition to the light-emitting layer 113, the hole-injection layer 111 and the hole-transport layer 112 in the hole-transport region 120 and the electron-transport layer 114 and the electron-injection layer 115 in the electron-transport region 121, and the structure as shown in FIG. 1B which includes a charge-generation layer 116 instead of the electron-injection layer 115 in FIG. 1A. Materials forming the layers are specifically described below.

The light-emitting layer 113 includes a light-emitting substance and a host material. The light-emitting layer 113 may additionally include other materials. Alternatively, the light-emitting layer 113 may be a stack of a plurality of layers with different compositions.

As the light-emitting substance, a fluorescent substance, a phosphorescent substance, a substance exhibiting thermally activated delayed fluorescence (TADF), or any of the other light-emitting substances may be used.

Examples of the material that can be used as a fluorescent substance in the light-emitting layer 113 are as follows. Other fluorescent substances can also be used.

The examples include 5,6-bis[4-(10-phenyl-9-anthryl)phenyl]-2,2′-bipyridine (abbreviation: PAP2BPy), 5,6-bis[4′-(10-phenyl-9-anthryl)biphenyl-4-yl]-2,2′-bipyridine (abbreviation: PAPP2BPy), N,N′-diphenyl-N,N′-bis[4-(9-phenyl-9H-fluoren-9-yl)phenyl]pyrene-1,6-diamine (abbreviation: 1,6FLPAPrn), N,N′-bis(3-methylphenyl)-N,N′-bis[3-(9-phenyl-9H-fluoren-9-yl)phenyl]pyrene-1,6-diamine (abbreviation: 1,6mMemFLPAPrn), N,N′-bis[4-(9H-carbazol-9-yl)phenyl]-N,N′-diphenylstilbene-4,4′-diamine (abbreviation: YGA2S), 4-(9H-carbazol-9-yl)-4′-(10-phenyl-9-anthryl)triphenylamine (abbreviation: YGAPA), 4-(9H-carbazol-9-yl)-4′-(9,10-diphenyl-2-anthryl)triphenylamine (abbreviation: 2YGAPPA), N,9-diphenyl-N-[4-(10-phenyl-9-anthryl)phenyl]-9H-carbazol-3-amine (abbreviation: PCAPA), perylene, 2,5,8,11-tetra-tert-butylperylene (abbreviation: TBP), 4-(10-phenyl-9-anthryl)-4′-(9-phenyl-9H-carbazol-3-yl)triphenylamine (abbreviation: PCBAPA), N,N″-(2-tert-butylanthracene-9,10-diyldi-4,1-phenylene)bis[N,N′,N′-triphenyl-1,4-phenylenediamine] (abbreviation: DPABPA), N,9-diphenyl-N-[4-(9,10-diphenyl-2-anthryl)phenyl]-9H-carbazol-3-amine (abbreviation: 2PCAPPA), N-[4-(9,10-diphenyl-2-anthryl)phenyl]-N,N′,N′-triphenyl-1,4-phenylenediamine (abbreviation: 2DPAPPA), N,N,N′,N′,N″,N″,N′″,N′″-octaphenyldibenzo[g,p]chrysene-2,7,10,15-tetraamine (abbreviation: DBC1), coumarin 30, N-(9,10-diphenyl-2-anthryl)-N,9-diphenyl-9H-carbazol-3-amine (abbreviation: 2PCAPA), N-[9,10-bis(1,1′-biphenyl-2-yl)-2-anthryl]-N,9-diphenyl-9H-carbazol-3-amine (abbreviation: 2PCABPhA), N-(9,10-diphenyl-2-anthryl)-N,N′,N′-triphenyl-1,4-phenylenediamine (abbreviation: 2DPAPA), N-[9,10-bis(1,1′-biphenyl-2-yl)-2-anthryl]-N,N′,N′-triphenyl-1,4-phenylenediamine (abbreviation: 2DPABPhA), 9,10-bis(1,1′-biphenyl-2-yl)-N-[4-(9H-carbazol-9-yl)phenyl]-N-phenylanthracen-2-amine (abbreviation: 2YGABPhA), N,N,9-triphenylanthracen-9-amine (abbreviation: DPhAPhA), coumarin 545T, N,N′-diphenylquinacridone (abbreviation: DPQd), rubrene, 5,12-bis(1,1′-biphenyl-4-yl)-6,11-diphenyltetracene (abbreviation: BPT), 2-(2-{2-[4-(dimethylamino)phenyl]ethenyl}-6-methyl-4H-pyran-4-ylidene)propanedinitrile (abbreviation: DCM1), 2-{2-methyl-6-[2-(2,3,6,7-tetrahydro-1H,5H-benzo[ij]quinolizin-9-yl)ethenyl]-4H-pyran-4-ylidene}propanedinitrile (abbreviation: DCM2), N,N,N′,N′-tetrakis(4-methylphenyl)tetracene-5,11-diamine (abbreviation: p-mPhTD), 7,14-diphenyl-N,N,N′,N′-tetrakis(4-methylphenyl)acenaphtho[1,2-a]fluoranthene-3,10-diamine (abbreviation: p-mPhAFD), 2-{2-isopropyl-6-[2-(1,1,7,7-tetramethyl-2,3,6,7-tetrahydro-1H,5H-benzo[ij]quinolizin-9-yl)ethenyl]-4H-pyran-4-ylidene}propanedinitrile (abbreviation: DCJTI), 2-{2-tert-butyl-6-[2-(1,1,7,7-tetramethyl-2,3,6,7-tetrahydro-1H,5H-benzo[ij]quinolizin-9-yl)ethenyl]-4H-pyran-4-ylidene}propanedinitrile (abbreviation: DCJTB), 2-(2,6-bis{2-[4-(dimethylamino)phenyl]ethenyl}-4H-pyran-4-ylidene)propanedinitrile (abbreviation: BisDCM), 2-{2,6-bis[2-(8-methoxy-1,1,7,7-tetramethyl-2,3,6,7-tetrahydro-1H,5H-benzo[ij]quinolizin-9-yl)ethenyl]-4H-pyran-4-ylidene}propanedinitrile (abbreviation: BisDCJTM), N,N′-diphenyl-N,N′-(1,6-pyrene-diyl)bis[(6-phenylbenzo[b]naphtho[1,2-d]furan)-8-amine] (abbreviation: 1,6BnfAPrn-03), 3,10-bis[N-(9-phenyl-9H-carbazol-2-yl)-N-phenylamino]naphtho[2,3-b;6,7-b′]bisbenzofuran (abbreviation: 3,10PCA2Nbf(IV)-02), and 3,10-bis[N-(dibenzofuran-3-yl)-N-phenylamino]naphtho[2,3-b;6,7-b′]bisbenzofuran (abbreviation: 3,10FrA2Nbf(IV)-02). Fused aromatic diamine compounds typified by pyrenediamine compounds such as 1,6FLPAPrn, 1,6mMemFLPAPrn, and 1,6BnfAPrn-03 are particularly preferable because of their high hole-trapping properties, high emission efficiency, and high reliability.

Examples of the material that can be used when a phosphorescent substance is used as the light-emitting substance in the light-emitting layer 113 are as follows.

The examples include an organometallic iridium complex having a 4H-triazole skeleton, such as tris{2-[5-(2-methylphenyl)-4-(2,6-dimethylphenyl)-4H-1,2,4-triazol-3-yl-κN2]phenyl-κC}iridium(III) (abbreviation: [Ir(mpptz-dmp)₃]), tris(5-methyl-3,4-diphenyl-4H-1,2,4-triazolato)iridium(III) (abbreviation: [Ir(Mptz)₃]), or tris[4-(3-biphenyl)-5-isopropyl-3-phenyl-4H-1,2,4-triazolato]iridium(III) (abbreviation: [Ir(iPrptz-3b)₃]); an organometallic iridium complex having a 1H-triazole skeleton, such as tris[3-methyl-1-(2-methylphenyl)-5-phenyl-1H-1,2,4-triazolato]iridium(III) (abbreviation: [Ir(Mptz1-mp)₃]) or tris(1-methyl-5-phenyl-3-propyl-1H-1,2,4-triazolato)iridium(III) (abbreviation: [Ir(Prptz1-Me)₃]); an organometallic iridium complex having an imidazole skeleton, such as fac-tris[(1-2,6-diisopropylphenyl)-2-phenyl-1H-imidazole]iridium(III) (abbreviation: [Ir(iPrpmi)₃]) or tris[3-(2,6-dimethylphenyl)-7-methylimidazo[1,2-f]phenanthridinato]iridium(III) (abbreviation: [Ir(dmpimpt-Me)₃]); and an organometallic iridium complex in which a phenylpyridine derivative having an electron-withdrawing group is a ligand, such as bis[2-(4′,6′-difluorophenyl)pyridinato-N,C^{2′}]iridium(III) tetrakis(1-pyrazolyl)borate (abbreviation: FIr6), bis[2-(4′,6′-difluorophenyl)pyridinato-N,C^{2′}]iridium(III) picolinate (abbreviation: FIrpic), bis{2-[3′,5′-bis(trifluoromethyl)phenyl]pyridinato-N,C^{2′}}iridium(III) picolinate (abbreviation: [Ir(CF₃ppy)₂(pic)]), or bis[2-(4′,6′-difluorophenyl)pyridinato-N,C^{2′}]iridium(III) acetylacetonate (abbreviation: FIracac). These compounds exhibit blue phosphorescence and have an emission peak in the wavelength range of 440 nm to 520 nm.

Other examples include an organometallic iridium complex having a pyrimidine skeleton, such as tris(4-methyl-6-phenylpyrimidinato)iridium(III) (abbreviation: [Ir(mppm)₃]), tris(4-tert-butyl6-phenylpyrimidinato)iridium(III) (abbreviation: [Ir(tBuppm)₃]), (acetylacetonato)bis(6-methyl-4-phenylpyrimidinato)iridium(III) (abbreviation: [Ir(mppm)₂(acac)]), (acetylacetonato)bis(6-tert-butyl-4-phenylpyrimidinato)iridium(III) (abbreviation: [Ir(tBuppm)₂(acac)]), (acetylacetonato)bis[6-(2-norbornyl)-4-phenylpyrimidinato]iridium(III) (abbreviation: [Ir(nbppm)₂(acac)]), (acetylacetonato)bis[5-methyl-6-(2-methylphenyl)-4-phenylpyrimidinato]iridium(III) (abbreviation: [Ir(mpmppm)₂(acac)]), or (acetylacetonato)bis(4,6-diphenylpyrimidinato)iridium(III) (abbreviation: [Ir(dppm)₂(acac)]); an organometallic iridium complex having a pyrazine skeleton, such as (acetylacetonato)bis(3,5-dimethyl-2-phenylpyrazinato)iridium(III) (abbreviation: [Ir(mppr-Me)₂(acac)]) or (acetylacetonato)bis(5-isopropyl-3-methyl-2-phenylpyrazinato)iridium(III) (abbreviation: [Ir(mppr-iPr)₂(acac)]); an organometallic iridium complex having a pyridine skeleton, such as tris(2-phenylpyridinato-N,C^{2′})iridium(III) (abbreviation: [Ir(ppy)₃]), bis(2-phenylpyridinato-N,C^{2′})iridium(III) acetylacetonate (abbreviation: [Ir(ppy)₂(acac)]), bis(benzo[h]quinolinato)iridium(III) acetylacetonate (abbreviation: [Ir(bzq)₂(acac)]), tris(benzo[h]quinolinato)iridium(III) (abbreviation: [Ir(bzq)₃]), tris(2-phenylquinolinato-N,C^{2′})iridium(III) (abbreviation: [Ir(pq)₃]), or bis(2-phenylquinolinato-N,C^{2′})iridium(III) acetylacetonate (abbreviation: [Ir(pq)₂(acac)]); and a rare earth metal complex such as tris(acetylacetonato)(monophenanthroline)terbium(III) (abbreviation: [Tb(acac)₃(Phen)]). These are mainly compounds that exhibit green phosphorescence and have an emission peak in the wavelength range of 500 nm to 600 nm. Note that an organometallic iridium complex having a pyrimidine skeleton is particularly preferable because of its distinctively high reliability and emission efficiency. It is particularly preferable that in the light-emitting device of one embodiment of the present invention, an iridium complex represented by Structural Formula (300) or Structural Formula (301) below be used as a light-emitting material. Since the iridium complex has an alkyl group, the iridium complex is easily dissolved in an organic solvent, which facilitates preparation of varnish.

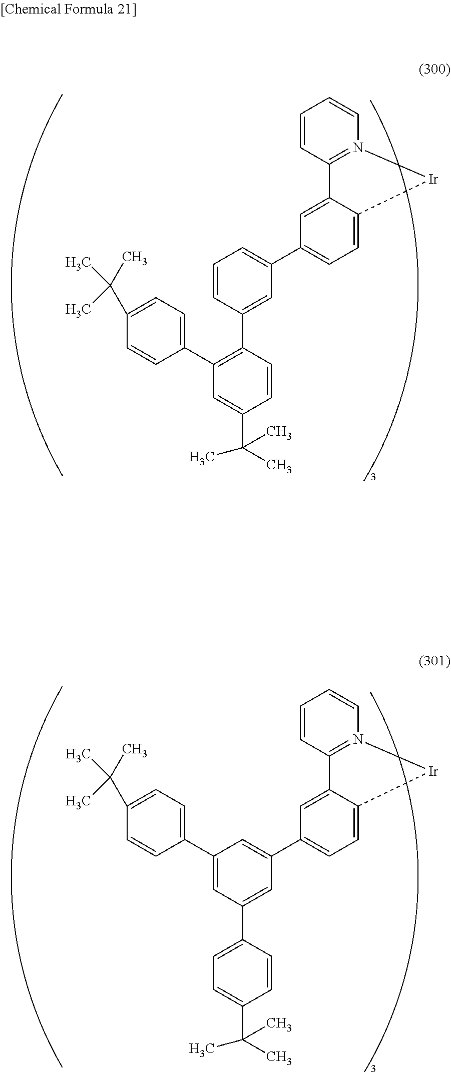

It is known that when a light-emitting layer that contains the iridium complex represented by Structural Formula (301) above is subjected to ToF-SIMS measurement, a signal at m/z=1676 and signals at m/z=1181 and m/z=685 indicating product ions appear in a positive mode.

Other examples include an organometallic iridium complex having a pyrimidine skeleton, such as (diisobutyrylmethanato)bis[4,6-bis(3-methylphenyl)pyrimidinato]iridium(III) (abbreviation: [Ir(5mdppm)₂(dibm)]), bis[4,6-bis(3-methylphenyl)pyrimidinato](dipivaloylmethanato)iridium(III) (abbreviation: [Ir(5mdppm)₂(dpm)]), or bis[4,6-di(naphthalen-1-yl)pyrimidinato](dipivaloylmethanato)iridium(III) (abbreviation: [Ir(d1npm)₂(dpm)]); an organometallic iridium complex having a pyrazine skeleton, such as (acetylacetonato)bis(2,3,5-triphenylpyrazinato)iridium(III) (abbreviation: [Ir(tppr)₂(acac)]), bis(2,3,5-triphenylpyrazinato)(dipivaloylmethanato)iridium(III) (abbreviation: [Ir(tppr)₂(dpm)]), or (acetylacetonato)bis[2,3-bis(4-fluorophenyl)quinoxalinato]iridium(III) (abbreviation: [Ir(Fdpq)₂(acac)]); an organometallic iridium complex having a pyridine skeleton, such as tris(1-phenylisoquinolinato-N,C^{2′})iridium(III) (abbreviation: [Ir(piq)₃]) or bis(1-phenylisoquinolinato-N,C^{2′})iridium(III) acetylacetonate (abbreviation: [Ir(piq)₂(acac)]); a platinum complex such as 2,3,7,8,12,13,17,18-octaethyl-21H,23H-porphyrinplatinum(II) (abbreviation: PtOEP); and a rare earth metal complex such as tris(1,3-diphenyl-1,3-propanedionato)(monophenanthroline)europium(III) (abbreviation: [Eu(DBM)₃(Phen)]) or tris[l-(2-thenoyl)-3,3,3-trifluoroacetonato](monophenanthroline)europium(III) (abbreviation: [Eu(TTA)₃(Phen)]). These compounds exhibit red phosphorescence and have an emission peak in the wavelength range of 600 nm to 700 nm. Organometallic iridium complexes having a pyrazine skeleton can provide red light emission with favorable chromaticity.

Besides the above phosphorescent compounds, known phosphorescent compounds may be selected and used.

Examples of the TADF material include a fullerene, a derivative thereof, an acridine, a derivative thereof, and an eosin derivative. Furthermore, a metal-containing porphyrin, such as a porphyrin containing magnesium (Mg), zinc (Zn), cadmium (Cd), tin (Sn), platinum (Pt), indium (In), or palladium (Pd), can be given as an example. Examples of the metal-containing porphyrin include a protoporphyrin-tin fluoride complex (SnF₂(Proto IX)), a mesoporphyrin-tin fluoride complex (SnF₂(Meso IX)), a hematoporphyrin-tin fluoride complex (SnF₂(Hemato IX)), a coproporphyrin tetramethyl ester-tin fluoride complex (SnF₂(Copro III-4Me)), an octaethylporphyrin-tin fluoride complex (SnF₂(OEP)), an etioporphyrin-tin fluoride complex (SnF₂(Etio I)), and an octaethylporphyrin-platinum chloride complex (PtCl₂OEP), which are represented by the following structural formulae.

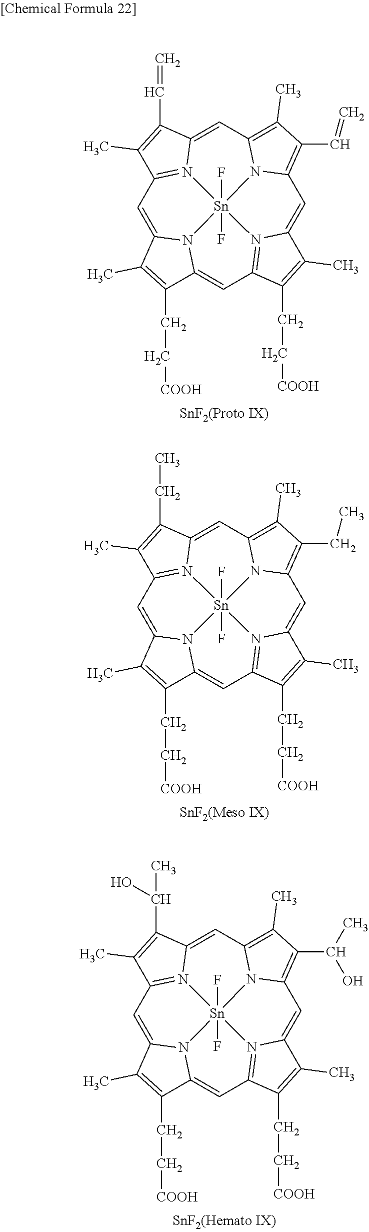

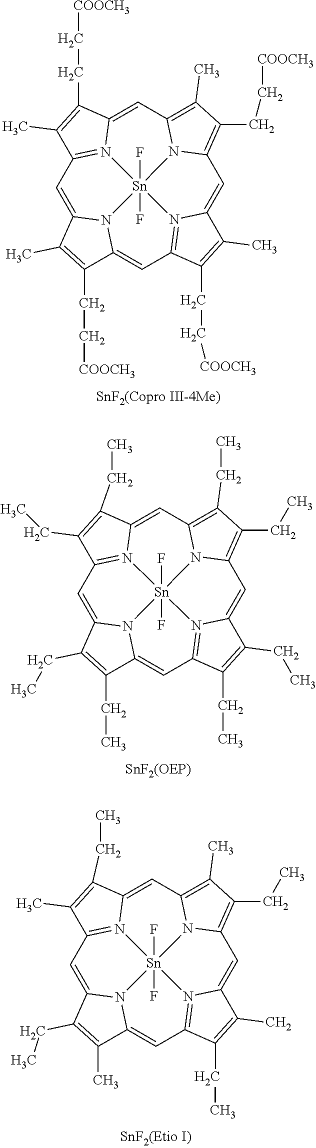

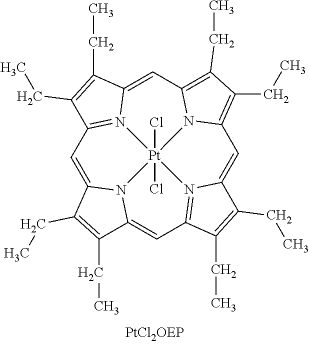

Alternatively, a heterocyclic compound having one or both of a π-electron rich heteroaromatic ring and a π-electron deficient heteroaromatic ring that is represented by any of the following structural formulae, such as 2-(biphenyl-4-yl)-4,6-bis(12-phenylindolo[2,3-a]carbazol-11-yl)-1,3,5-triazine (abbreviation: PIC-TRZ), 9-(4,6-diphenyl-1,3,5-triazin-2-yl)-9′-phenyl-9H,9′H-3,3′-bicarbazole (abbreviation: PCCzTzn), 9-[4-(4,6-diphenyl-1,3,5-triazin-2-yl)phenyl]-9′-phenyl-9H,9′H-3,3′-bicarbazole (abbreviation: PCCzPTzn), 2-[4-(10H-phenoxazin-10-yl)phenyl]-4,6-diphenyl-1,3,5-triazine (abbreviation: PXZ-TRZ), 3-[4-(5-phenyl-5,10-dihydrophenazin-10-yl)phenyl]-4,5-diphenyl-1,2,4-triazole (abbreviation: PPZ-3TPT), 3-(9,9-dimethyl-9H-acridin-10-yl)-9H-xanthen-9-one (abbreviation: ACRXTN), bis[4-(9,9-dimethyl-9,10-dihydroacridine)phenyl]sulfone (abbreviation: DMAC-DPS), or 10-phenyl-10H,10′H-spiro[acridin-9,9′-anthracen]-10′-one (abbreviation: ACRSA), can be used. Such a heterocyclic compound is preferable because of having excellent electron-transport and hole-transport properties owing to a π-electron rich heteroaromatic ring and a π-electron deficient heteroaromatic ring. Among skeletons having a π-electron deficient heteroaromatic ring, a pyridine skeleton, a diazine skeleton (a pyrimidine skeleton, a pyrazine skeleton, and a pyridazine skeleton), and a triazine skeleton are preferred because of their high stability and reliability. In particular, a benzofuropyrimidine skeleton, a benzothienopyrimidine skeleton, a benzofuropyrazine skeleton, and a benzothienopyrazine skeleton are preferred because of their high acceptor properties and high reliability. Among skeletons having a π-electron rich heteroaromatic ring, an acridine skeleton, a phenoxazine skeleton, a phenothiazine skeleton, a furan skeleton, a thiophene skeleton, and a pyrrole skeleton have high stability and reliability; thus, at least one of these skeletons is preferably included. A dibenzofuran skeleton is preferable as a furan skeleton, and a dibenzothiophene skeleton is preferable as a thiophene skeleton. As a pyrrole skeleton, an indole skeleton, a carbazole skeleton, an indolocarbazole skeleton, a bicarbazole skeleton, and a 3-(9-phenyl-9H-carbazol-3-yl)-9H-carbazole skeleton are particularly preferable. Note that a substance in which a π-electron rich heteroaromatic ring is directly bonded to a π-electron deficient heteroaromatic ring is particularly preferred because the electron-donating property of the π-electron rich heteroaromatic ring and the electron-accepting property of the π-electron deficient heteroaromatic ring are both enhanced, the energy difference between the S1 level and the T1 level becomes small, and thus thermally activated delayed fluorescence can be obtained with high efficiency. Note that an aromatic ring to which an electron-withdrawing group such as a cyano group is bonded may be used instead of the π-electron deficient heteroaromatic ring. As a π-electron rich skeleton, an aromatic amine skeleton, a phenazine skeleton, or the like can be used. As a π-electron deficient skeleton, a xanthene skeleton, a thioxanthene dioxide skeleton, an oxadiazole skeleton, a triazole skeleton, an imidazole skeleton, an anthraquinone skeleton, a skeleton containing boron such as phenylborane or boranthrene, an aromatic ring or a heteroaromatic ring having a nitrile group or a cyano group such as benzonitrile or cyanobenzene, a carbonyl skeleton such as benzophenone, a phosphine oxide skeleton, a sulfone skeleton, or the like can be used. As described above, a π-electron deficient skeleton and a π-electron rich skeleton can be used instead of at least one of the π-electron deficient heteroaromatic ring and the π-electron rich heteroaromatic ring.

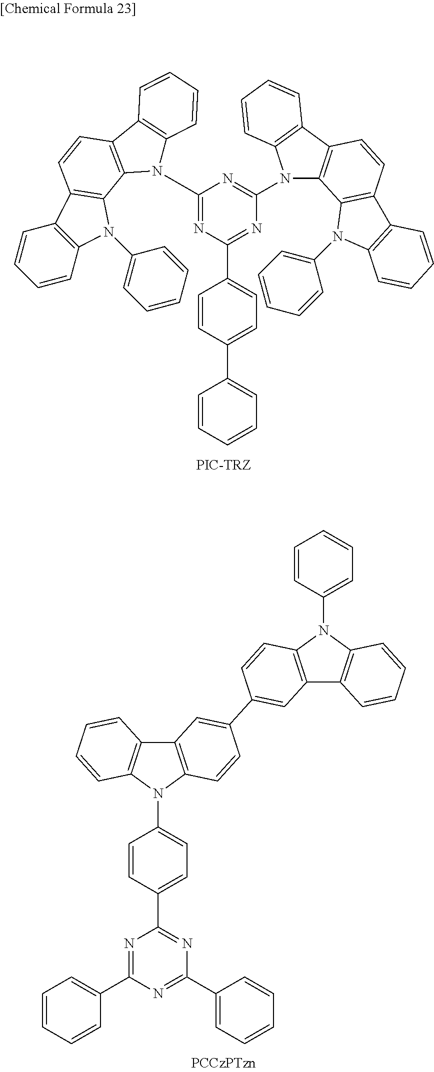 [SMILES: c1ccc(-c2ccc(-c3nc(-n4c5ccccc5c5ccc6c7ccccc7n(-c7ccccc7)c6c54)nc(-n4c5ccccc5c5ccc6c7ccccc7n(-c7ccccc7)c6c54)n3)cc2)cc1 ; c1ccc(-c2nc(-c3ccccc3)nc(-c3ccc(-n4c5ccccc5c5cc(-c6ccc7c(c6)c6ccccc6n7-c6ccccc6)ccc54)cc3)n2)cc1]

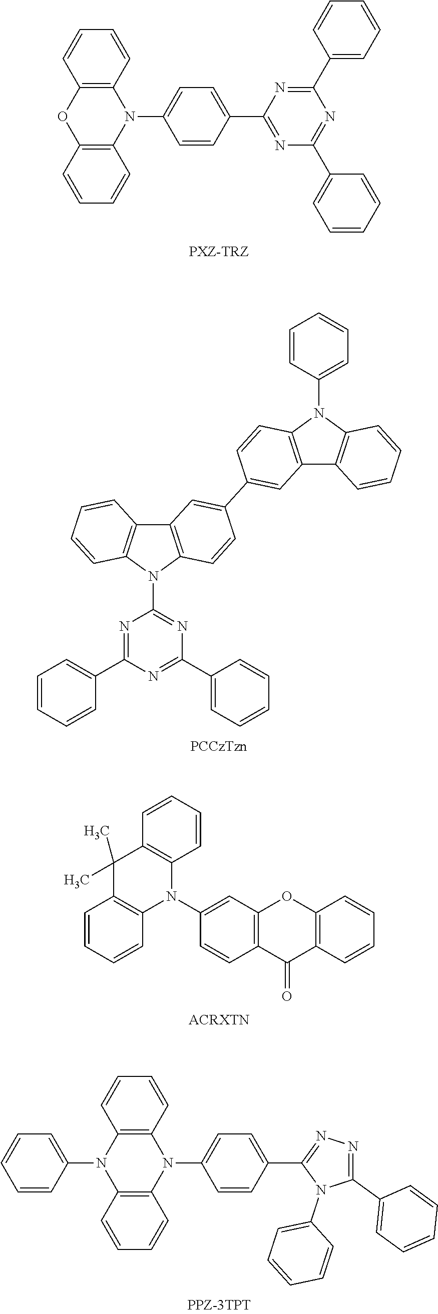 [SMILES: c1ccc(-c2nc(-c3ccccc3)nc(-c3ccc(N4c5ccccc5Oc5ccccc54)cc3)n2)cc1 ; c1ccc(-c2nc(-c3ccccc3)nc(-n3c4ccccc4c4cc(-c5ccc6c(c5)c5ccccc5n6-c5ccccc5)ccc43)n2)cc1 ; CC1(C)c2ccccc2N(c2ccc3c(=O)c4ccccc4oc3c2)c2ccccc21 ; c1ccc(-c2nnc(-c3ccc(N4c5ccccc5N(c5ccccc5)c5ccccc54)cc3)n2-c2ccccc2)cc1]

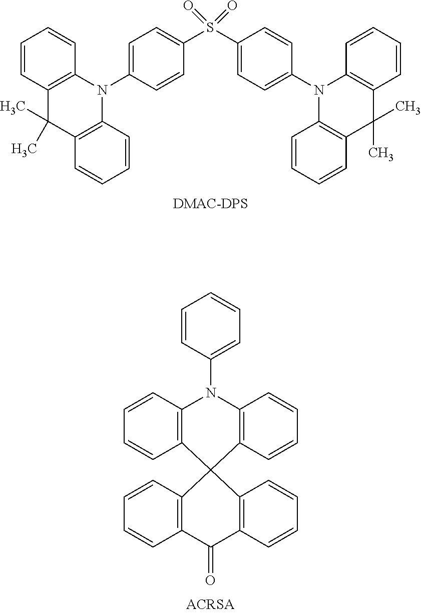 [SMILES: CC1(C)c2ccccc2N(c2ccc(S(=O)(=O)c3ccc(N4c5ccccc5C(C)(C)c5ccccc54)cc3)cc2)c2ccccc21 ; O=C1c2ccccc2C2(c3ccccc31)c1ccccc1N(c1ccccc1)c1ccccc12]

Note that a TADF material is a material having a small difference between the S1 level and the T1 level and a function of converting triplet excitation energy into singlet excitation energy by reverse intersystem crossing. Thus, a TADF material can upconvert triplet excitation energy into singlet excitation energy (i.e., reverse intersystem crossing) using a small amount of thermal energy and efficiently generate a singlet excited state. In addition, the triplet excitation energy can be converted into light.

An exciplex whose excited state is formed by two kinds of substances has an extremely small difference between the S1 level and the T1 level and functions as a TADF material capable of converting triplet excitation energy into singlet excitation energy.

A phosphorescent spectrum observed at a low temperature (e.g., 77 K to 10 K) is used for an index of the T1 level. When the level of energy with a wavelength of the line obtained by extrapolating a tangent to the fluorescent spectrum at a tail on the short wavelength side is the S1 level and the level of energy with a wavelength of the line obtained by extrapolating a tangent to the phosphorescent spectrum at a tail on the short wavelength side is the T1 level, the difference between the S1 level and the T1 level of the TADF material is preferably smaller than or equal to 0.3 eV, further preferably smaller than or equal to 0.2 eV.

When a TADF material is used as the light-emitting substance, the S1 level of the host material is preferably higher than that of the TADF material. In addition, the T1 level of the host material is preferably higher than that of the TADF material.

As the host material in the light-emitting layer, various carrier-transport materials such as materials having an electron-transport property, materials having a hole-transport property, and the above-described TADF materials can be used.

The material having a hole-transport property preferably has a hole mobility of 1×10⁻⁶ cm²/Vs or more. The material having a hole-transport property is particularly preferably an organic compound having an amine skeleton or a π-electron rich heteroaromatic ring, examples of which include a compound having an aromatic amine skeleton, such as 4,4′-bis[N-(1-naphthyl)-N-phenylamino]biphenyl (abbreviation: NPB), N,N′-bis(3-methylphenyl)-N,N′-diphenyl-[1,1′-biphenyl]-4,4′-diamine (abbreviation: TPD), 4,4′-bis[N-(spiro-9,9′-bifluoren-2-yl)-N-phenylamino]biphenyl (abbreviation: BSPB), 4-phenyl-4′-(9-phenylfluoren-9-yl)triphenylamine (abbreviation: BPAFLP), 4-phenyl-3′-(9-phenylfluoren-9-yl)triphenylamine (abbreviation: mBPAFLP), 4-phenyl-4′-(9-phenyl-9H-carbazol-3-yl)triphenylamine (abbreviation: PCBA1BP), 4,4′-diphenyl-4″-(9-phenyl-9H-carbazol-3-yl)triphenylamine (abbreviation: PCBBi1BP), 4-(1-naphthyl)-4′-(9-phenyl-9H-carbazol-3-yl)triphenylamine (abbreviation: PCBANB), 4,4′-di(1-naphthyl)-4″-(9-phenyl-9H-carbazol-3-yl)triphenylamine (abbreviation: PCBNBB), 9,9-dimethyl-N-phenyl-N-[4-(9-phenyl-9H-carbazol-3-yl)phenyl]fluoren-2-amine (abbreviation: PCBAF), or N-phenyl-N-[4-(9-phenyl-9H-carbazol-3-yl)phenyl]-9,9′-spirobi[9H-fluoren]-2-amine (abbreviation: PCBASF); a compound having a carbazole skeleton, such as 1,3-bis(N-carbazolyl)benzene (abbreviation: mCP), 4,4′-di(N-carbazolyl)biphenyl (abbreviation: CBP), 3,6-bis(3,5-diphenylphenyl)-9-phenylcarbazole (abbreviation: CzTP), or 3,3′-bis(9-phenyl-9H-carbazole) (abbreviation: PCCP); a compound having a thiophene skeleton, such as 4,4′,4″-(benzene-1,3,5-triyl)tri(dibenzothiophene) (abbreviation: DBT3P-II), 2,8-diphenyl-4-[4-(9-phenyl-9H-fluoren-9-yl)phenyl]dibenzothiophene (abbreviation: DBTFLP-III), or 4-[4-(9-phenyl-9H-fluoren-9-yl)phenyl]-6-phenyldibenzothiophene (abbreviation: DBTFLP-IV); and a compound having a furan skeleton, such as 4,4′,4″-(benzene-1,3,5-triyl)tri(dibenzofuran) (abbreviation: DBF3P-II) or 4-{3-[3-(9-phenyl-9H-fluoren-9-yl)phenyl]phenyl}dibenzofuran (abbreviation: mmDBFFLBi-II). Among the above materials, the compound having an aromatic amine skeleton and the compound having a carbazole skeleton are preferable because these compounds are highly reliable and have high hole-transport properties to contribute to a reduction in driving voltage.

In addition, N-(4-biphenyl)-6,N-diphenylbenzo[b]naphtho[1,2-d]furan-8-amine (abbreviation: BnfABP), N,N-bis(4-biphenyl)-6-phenylbenzo[b]naphtho[1,2-d]furan-8-amine (abbreviation: BBABnf), 4,4′-bis(6-phenylbenzo[b]naphtho[1,2-d]furan-8-yl)-4″-phenyltriphenylamine (abbreviation: BnfBB1BP), N,N-bis(4-biphenyl)benzo[b]naphtho[1,2-d]furan-6-amine (abbreviation: BBABnf(6)), N,N-bis(4-biphenyl)benzo[b]naphtho[1,2-d]furan-8-amine (abbreviation: BBABnf(8)), N,N-bis(4-biphenyl)benzo[b]naphtho[2,3-d]furan-4-amine (abbreviation: BBABnf(II)(4)), N,N-bis[4-(dibenzofuran-4-yl)phenyl]-4-amino-p-terphenyl (abbreviation: DBfBB1TP), N-[4-(dibenzothiophen-4-yl)phenyl]-N-phenyl-4-biphenylamine (abbreviation: ThBA1BP), 4-(2-naphthyl)-4′,4″-diphenyltriphenylamine (abbreviation: BBAβNB), 4-[4-(2-naphthyl)phenyl]-4′,4″-diphenyltriphenylamine (abbreviation: BBAβNBi), 4,4′-diphenyl-4″-(6;1′-binaphthyl-2-yl)triphenylamine (abbreviation: BBAαNβNB), 4,4′-diphenyl-4″-(7;1′-binaphthyl-2-yl)triphenylamine (abbreviation: BBAαNβNB-03), 4,4′-diphenyl-4″-(7-phenyl)naphthyl-2-yltriphenylamine (abbreviation: BBAPβNB-03), 4,4′-diphenyl-4″-(6;2′-binaphthyl-2-yl)triphenylamine (abbreviation: BBA(βN2)B), 4,4′-diphenyl-4″-(7;2′-binaphthyl-2-yl)triphenylamine (abbreviation: BBA(βN2)B-03), 4,4′-diphenyl-4″-(4;2′-binaphthyl-1-yl)triphenylamine (abbreviation: BBAβNαNB), 4,4′-diphenyl-4″-(5;2′-binaphthyl-1-yl)triphenylamine (abbreviation: BBAβNαNB-02), 4-(4-biphenylyl)-4′-(2-naphthyl)-4″-phenyltriphenylamine (abbreviation: TPBiAβNB), 4-(3-biphenylyl)-4′-[4-(2-naphthyl)phenyl]-4″-phenyltriphenylamine (abbreviation: mTPBiAβNBi), 4-(4-biphenylyl)-4′-[4-(2-naphthyl)phenyl]-4″-phenyltriphenylamine (abbreviation: TPBiAβNBi), 4-phenyl-4′-(1-naphthyl)triphenylamine (abbreviation: αNBA1BP), 4,4′-bis(1-naphthyl)triphenylamine (abbreviation: αNBB1BP), 4,4′-diphenyl-4″-[4′-(carbazol-9-yl)biphenyl-4-yl]triphenylamine (abbreviation: YGTBi1BP), 4′-[4-(3-phenyl-9H-carbazol-9-yl)phenyl]tris(1,1′-biphenyl-4-yl)amine (abbreviation: YGTBi1BP-02), 4-[4′-(carbazol-9-yl)biphenyl-4-yl]-4′-(2-naphthyl)-4″-phenyltriphenylamine (abbreviation: YGTBiβNB), N-[4-(9-phenyl-9H-carbazol-3-yl)phenyl]-N-[4-(1-naphthyl)phenyl]-9,9′-spirobi(9H-fluoren)-2-amine (abbreviation: PCBNBSF), N,N-bis(4-biphenylyl)-9,9′-spirobi[9H-fluoren]-2-amine (abbreviation: BBASF), N,N-bis([1,1′-biphenyl]-4-yl)-9,9′-spirobi[9H-fluoren]-4-amine (abbreviation: BBASF(4)), N-(1,1′-biphenyl-2-yl)-N-(9,9-dimethyl-9H-fluoren-2-yl)-9,9′-spirobi(9H-fluoren)-4-amine (abbreviation: oFBiSF), N-(4-biphenyl)-N-(dibenzofuran-4-yl)-9,9-dimethyl-9H-fluoren-2-amine (abbreviation: FrBiF), N-[4-(1-naphthyl)phenyl]-N-[3-(6-phenyldibenzofuran-4-yl)phenyl]-1-naphthylamine (abbreviation: mPDBfBNBN), 4-phenyl-4′-(9-phenylfluoren-9-yl)triphenylamine (abbreviation: BPAFLP), 4-phenyl-3′-(9-phenylfluoren-9-yl)triphenylamine (abbreviation: mBPAFLP), 4-phenyl-4′-[4-(9-phenylfluoren-9-yl)phenyl]triphenylamine (abbreviation: BPAFLBi), 4,4′-di(1-naphthyl)-4″-(9-phenyl-9H-carbazol-3-yl)triphenylamine (abbreviation: PCBNBB), N-phenyl-N-[4-(9-phenyl-9H-carbazol-3-yl)phenyl]-9,9′-spirobi[9H-fluoren]-2-amine (abbreviation: PCBASF), N-(1,1′-biphenyl-4-yl)-9,9-dimethyl-N-[4-(9-phenyl-9H-carbazol-3-yl)phenyl]-9H-fluoren-2-amine (abbreviation: PCBBiF), N,N-bis(9,9-dimethyl-9H-fluoren-2-yl)-9,9′-spirobi-9H-fluoren-4-amine, N,N-bis(9,9-dimethyl-9H-fluoren-2-yl)-9,9′-spirobi-9H-fluoren-3-amine, N,N-bis(9,9-dimethyl-9H-fluoren-2-yl)-9,9′-spirobi-9H-fluoren-2-amine, N,N-bis(9,9-dimethyl-9H-fluoren-2-yl)-9,9′-spirobi-9H-fluoren-1-amine, or the like can also be suitably used.

As the material having an electron-transport property, for example, a metal complex such as bis(10-hydroxybenzo[h]quinolinato)beryllium(II) (abbreviation: BeBq₂), bis(2-methyl-8-quinolinolato)(4-phenylphenolato)aluminum(III) (abbreviation: BAlq), bis(8-quinolinolato)zinc(II) (abbreviation: Znq), bis[2-(2-benzoxazolyl)phenolato]zinc(II) (abbreviation: ZnPBO), or bis[2-(2-benzothiazolyl)phenolato]zinc(II) (abbreviation: ZnBTZ); or an organic compound having a π-electron deficient heteroaromatic ring is preferable. Examples of the organic compound having a π-electron deficient heteroaromatic ring include a heterocyclic compound having a polyazole skeleton, such as 2-(4-biphenylyl)-5-(4-tert-butylphenyl)-1,3,4-oxadiazole (abbreviation: PBD), 3-(4-biphenylyl)-4-phenyl-5-(4-tert-butylphenyl)-1,2,4-triazole (abbreviation: TAZ), 1,3-bis[5-(p-tert-butylphenyl)-1,3,4-oxadiazol-2-yl]benzene (abbreviation: OXD-7), 9-[4-(5-phenyl-1,3,4-oxadiazol-2-yl)phenyl]-9H-carbazole (abbreviation: CO11), 2,2′,2″-(1,3,5-benzenetriyl)tris(1-phenyl-1H-benzimidazole) (abbreviation: TPBI), or 2-[3-(dibenzothiophen-4-yl)phenyl]-1-phenyl-1H-benzimidazole (abbreviation: mDBTBIm-II); a heterocyclic compound having a diazine skeleton, such as 2-[3-(dibenzothiophen-4-yl)phenyl]dibenzo[f,h]quinoxaline (abbreviation: 2mDBTPDBq-II), 2-[3′-(dibenzothiophen-4-yl)biphenyl-3-yl]dibenzo[f,h]quinoxaline (abbreviation: 2mDBTBPDBq-II), 2-[3′-(9H-carbazol-9-yl)biphenyl-3-yl]dibenzo[f,h]quinoxaline (abbreviation: 2mCzBPDBq), 4,6-bis[3-(phenanthren-9-yl)phenyl]pyrimidine (abbreviation: 4,6mPnP2Pm), or 4,6-bis[3-(4-dibenzothienyl)phenyl]pyrimidine (abbreviation: 4,6mDBTP2Pm-II); a heterocyclic compound having a pyridine skeleton, such as 3,5-bis[3-(9H-carbazol-9-yl)phenyl]pyridine (abbreviation: 35DCzPPy) or 1,3,5-tri[3-(3-pyridyl)phenyl]benzene (abbreviation: TmPyPB); and a heterocyclic compound having a triazine skeleton, such as 2-[3′-(9,9-dimethyl-9H-fluoren-2-yl)-1,1′-biphenyl-3-yl]-4,6-diphenyl-1,3,5-triazine (abbreviation: mFBPTzn), 2-[(1,1′-biphenyl)-4-yl]-4-phenyl-6-[9,9′-spirobi(9H-fluoren)-2-yl]-1,3,5-triazine (abbreviation: BP-SFTzn), 2-{3-[3-(benzo[b]naphtho[1,2-d]furan-8-yl)phenyl]phenyl}-4,6-diphenyl-1,3,5-triazine (abbreviation: mBnfBPTzn), or 2-{3-[3-(benzo[b]naphtho[1,2-d]furan-6-yl)phenyl]phenyl}-4,6-diphenyl-1,3,5-triazine (abbreviation: mBnfBPTzn-02). Among the above materials, the heterocyclic compound having a diazine skeleton, the heterocyclic compound having a pyridine skeleton, and the heterocyclic compound having a triazine skeleton have high reliability and thus are preferable. In particular, the heterocyclic compound having a diazine (pyrimidine or pyrazine) skeleton and the heterocyclic compound having a triazine skeleton have a high electron-transport property to contribute to a reduction in driving voltage.

As the TADF material that can be used as the host material, the above materials mentioned as the TADF material can also be used. When the TADF material is used as the host material, triplet excitation energy generated in the TADF material is converted into singlet excitation energy by reverse intersystem crossing and transferred to the light-emitting substance, whereby the emission efficiency of the light-emitting device can be increased. Here, the TADF material functions as an energy donor, and the light-emitting substance functions as an energy acceptor.

This is very effective in the case where the light-emitting substance is a fluorescent substance. In that case, the S1 level of the TADF material is preferably higher than that of the fluorescent substance in order that high emission efficiency can be achieved. Furthermore, the T1 level of the TADF material is preferably higher than the S1 level of the fluorescent substance. Therefore, the T1 level of the TADF material is preferably higher than that of the fluorescent substance.

It is also preferable to use a TADF material that emits light whose wavelength overlaps with the wavelength of a lowest-energy-side absorption band of the fluorescent substance, in which case excitation energy is transferred smoothly from the TADF material to the fluorescent substance and light emission can be obtained efficiently.

In addition, in order to efficiently generate singlet excitation energy from the triplet excitation energy by reverse intersystem crossing, carrier recombination preferably occurs in the TADF material. It is also preferable that the triplet excitation energy generated in the TADF material not be transferred to the triplet excitation energy of the fluorescent substance. For that reason, the fluorescent substance preferably has a protective group around a luminophore (a skeleton which causes light emission) of the fluorescent substance. As the protective group, a substituent having no π bond and a saturated hydrocarbon are preferably used. Specific examples include an alkyl group having 3 to 10 carbon atoms, a substituted or unsubstituted cycloalkyl group having 3 to 10 carbon atoms, and a trialkylsilyl group having 3 to 10 carbon atoms. It is further preferable that the fluorescent substance have a plurality of protective groups. The substituents having no π bond are poor in carrier transport performance, whereby the TADF material and the luminophore of the fluorescent substance can be distanced from each other with little influence on carrier transport or carrier recombination. Here, the luminophore refers to an atomic group (skeleton) that causes light emission in a fluorescent substance. The luminophore is preferably a skeleton having a π bond, further preferably includes an aromatic ring, and still further preferably includes a fused aromatic ring or a fused heteroaromatic ring. Examples of the fused aromatic ring or the fused heteroaromatic ring include a phenanthrene skeleton, a stilbene skeleton, an acridone skeleton, a phenoxazine skeleton, and a phenothiazine skeleton. Specifically, a fluorescent substance having any of a naphthalene skeleton, an anthracene skeleton, a fluorene skeleton, a chrysene skeleton, a triphenylene skeleton, a tetracene skeleton, a pyrene skeleton, a perylene skeleton, a coumarin skeleton, a quinacridone skeleton, and a naphthobisbenzofuran skeleton is preferred because of its high fluorescence quantum yield.

In the case where a fluorescent substance is used as the light-emitting substance, a material having an anthracene skeleton is suitably used as the host material. The use of a substance having an anthracene skeleton as the host material for the fluorescent substance makes it possible to obtain a light-emitting layer with high emission efficiency and high durability. Among the substances having an anthracene skeleton, a substance having a diphenylanthracene skeleton, in particular, a substance having a 9,10-diphenylanthracene skeleton, is chemically stable and thus is preferably used as the host material. The host material preferably has a carbazole skeleton, in which case the hole-injection and hole-transport properties are improved; further preferably, the host material has a benzocarbazole skeleton in which a benzene ring is further fused to carbazole because the HOMO level thereof is shallower than that of carbazole by approximately 0.1 eV and thus holes enter the host material easily. In particular, the host material preferably has a dibenzocarbazole skeleton, in which case the HOMO level thereof is shallower than that of carbazole by approximately 0.1 eV so that holes enter the host material easily, the hole-transport property is improved, and the heat resistance is increased. Accordingly, a substance that has both a 9,10-diphenylanthracene skeleton and a carbazole skeleton (or a benzocarbazole or dibenzocarbazole skeleton) is further preferable as the host material. Note that in terms of the hole-injection and hole-transport properties described above, instead of a carbazole skeleton, a benzofluorene skeleton or a dibenzofluorene skeleton may be used. Examples of such a substance include 9-phenyl-3-[4-(10-phenyl-9-anthryl)phenyl]-9H-carbazole (abbreviation: PCzPA), 3-[4-(1-naphthyl)-phenyl]-9-phenyl-9H-carbazole (abbreviation: PCPN), 9-[4-(10-phenyl-9-anthracenyl)phenyl]-9H-carbazole (abbreviation: CzPA), 7-[4-(10-phenyl-9-anthryl)phenyl]-7H-dibenzo[c,g]carbazole (abbreviation: cgDBCzPA), 6-[3-(9,10-diphenyl-2-anthryl)phenyl]-benzo[b]naphtho[1,2-d]furan (abbreviation: 2mBnfPPA), 9-phenyl-10-{4-(9-phenyl-9H-fluoren-9-yl)biphenyl-4′-yl}anthracene (abbreviation: FLPPA), and 9-(1-naphthyl)-10-[4-(2-naphthyl)phenyl]anthracene (abbreviation: αN-βNPAnth). In particular, CzPA, cgDBCzPA, 2mBnfPPA, and PCzPA exhibit excellent properties and thus are preferably selected.

Note that the host material may be a mixture of a plurality of kinds of substances; in the case of using a mixed host material, it is preferable to mix a material having an electron-transport property with a material having a hole-transport property. By mixing the material having an electron-transport property with the material having a hole-transport property, the transport property of the light-emitting layer 113 can be easily adjusted and a recombination region can be easily controlled. The weight ratio of the content of the material having a hole-transport property to the content of the material having an electron-transport property may be 1:19 to 19:1.

Note that a phosphorescent substance can be used as part of the mixed material. When a fluorescent substance is used as the light-emitting substance, a phosphorescent substance can be used as an energy donor for supplying excitation energy to the fluorescent substance.

An exciplex may be formed of these mixed materials. These mixed materials are preferably selected so as to form an exciplex that exhibits light emission whose wavelength overlaps with the wavelength of a lowest-energy-side absorption band of the light-emitting substance, in which case energy can be transferred smoothly and light emission can be obtained efficiently. Such a structure is preferably employed to reduce the driving voltage.

Note that at least one of the materials forming an exciplex may be a phosphorescent substance. In this case, triplet excitation energy can be efficiently converted into singlet excitation energy by reverse intersystem crossing.

Combination of a material having an electron-transport property and a material having a hole-transport property whose HOMO level is higher than or equal to that of the material having an electron-transport property is preferable for forming an exciplex efficiently. In addition, the LUMO level of the material having a hole-transport property is preferably higher than or equal to that of the material having an electron-transport property. Note that the LUMO levels and the HOMO levels of the materials can be derived from the electrochemical characteristics (the reduction potentials and the oxidation potentials) of the materials that are measured by cyclic voltammetry (CV).

The formation of an exciplex can be confirmed by a phenomenon in which the emission spectrum of the mixed film in which the material having a hole-transport property and the material having an electron-transport property are mixed is shifted to the longer wavelength side than the emission spectrum of each of the materials (or has another peak on the longer wavelength side) observed by comparison of the emission spectra of the material having a hole-transport property, the material having an electron-transport property, and the mixed film of these materials, for example. Alternatively, the formation of an exciplex can be confirmed by a difference in transient response, such as a phenomenon in which the transient PL lifetime of the mixed film has longer lifetime components or has a larger proportion of delayed components than that of each of the materials, observed by comparison of transient photoluminescence (PL) of the material having a hole-transport property, the material having an electron-transport property, and the mixed film of these materials. The transient PL can be rephrased as transient electroluminescence (EL). That is, the formation of an exciplex can also be confirmed by a difference in transient response observed by comparison of the transient EL of the material having a hole-transport property, the material having an electron-transport property, and the mixed film of these materials.

Since the electron-transport layer 114 with the structure of the present invention can have a low refractive index, a layer with a low refractive index can be formed in the EL layer 103 without a significant decrease in driving voltage, leading to higher external quantum efficiency of the light-emitting device.

Note that the electron-transport layer 114 having this structure also serves as the electron-injection layer 115 in some cases.

There is preferably a difference in the concentration (including 0) of the alkali metal or the metal complex of the alkali metal in the electron-transport layer 114 in the thickness direction.

A layer including an alkali metal, an alkaline earth metal, a compound thereof, or a complex thereof such as lithium fluoride (LiF), cesium fluoride (CsF), calcium fluoride (CaF₂), or 8-quinolinolato-lithium (abbreviation: Liq) may be provided as the electron-injection layer 115 between the electron-transport layer 114 and the cathode 102. For example, an electride or a layer that is formed using a substance having an electron-transport property and that includes an alkali metal, an alkaline earth metal, or a compound thereof can be used as the electron-injection layer 115. Examples of the electride include a substance in which electrons are added at high concentration to a mixed oxide of calcium and aluminum.

Sodium fluoride is preferably used to improve the electron-transport property and water resistance of the light-emitting device. When an electron-injection layer of a light-emitting device including sodium fluoride in the electron-injection layer 115 is subjected to ToF-SIMS analysis, signals are observed which are attributed to anions or cations such as Na₂F⁺, NaF₂⁻, and Na₂F₃⁻ being different in the number of bonds between sodium and fluorine.

Furthermore, a layer containing an alkaline earth metal such as barium may be provided in contact with the cathode. This structure is preferably employed to make the property of electron injection from the cathode favorable.

The above layer containing barium may also include a heteroaromatic compound. The heteroaromatic compound is preferably an organic compound having a phenanthroline skeleton, particularly preferably 2-phenyl-9-[3-(9-phenyl-1,10-phenanthrolin-2-yl)phenyl]-1,10-phenanthroline represented by Structural Formula (400) below, for example.

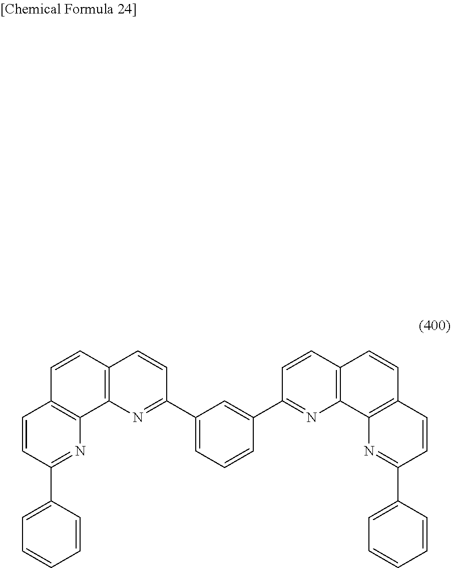 [SMILES: c1ccc(-c2ccc3ccc4ccc(-c5cccc(-c6ccc7ccc8ccc(-c9ccccc9)nc8c7n6)c5)nc4c3n2)cc1]

When a layer containing 2-phenyl-9-[3-(9-phenyl-1,10-phenanthrolin-2-yl)phenyl]-1,10-phenanthroline is subjected to ToF-SIMS analysis, a signal is observed at m/z=587 in both a positive mode and a negative mode. In the case where a film of the same material is formed and the same layer or a layer in contact with the layer contains an alkali metal, an alkaline earth metal, or a compound thereof, an ion of an alkali metal complex (e.g., m/z=609 in the case of a Na complex), an alkaline earth metal complex (e.g., m/z=724 in the case of a Ba complex), or the like is sometimes detected.

Note that as the electron-injection layer 115, it is possible to use a layer including a substance that has an electron-transport property (preferably an organic compound having a bipyridine skeleton) and includes a fluoride of the alkali metal or the alkaline earth metal at a concentration higher than or equal to that at which the electron-injection layer 115 becomes in a microcrystalline state (50 wt % or higher). Since the layer has a low refractive index, a light-emitting device including the layer can have high external quantum efficiency.

Instead of the electron-injection layer 115 in FIG. 1A, the charge-generation layer 116 may be provided (FIG. 1B). The charge-generation layer 116 refers to a layer capable of injecting holes into a layer in contact with the cathode side of the charge-generation layer and electrons into a layer in contact with the anode side thereof when a potential is applied. The charge-generation layer 116 includes at least a p-type layer 117. The p-type layer 117 is preferably formed using any of the composite materials given above as examples of materials that can be used for the hole-injection layer 111. The p-type layer 117 may be formed by stacking a film including the above-described acceptor material as a material included in the composite material and a film including a hole-transport material. When a potential is applied to the p-type layer 117, electrons are injected into the electron-transport layer 114 and holes are injected into the cathode 102; thus, the light-emitting device operates.

Note that the charge-generation layer 116 preferably includes one or both of an electron-relay layer 118 and an electron-injection buffer layer 119 in addition to the p-type layer 117.

The electron-relay layer 118 includes at least the substance with an electron-transport property and has a function of preventing an interaction between the electron-injection buffer layer 119 and the p-type layer 117 and smoothly transferring electrons. The LUMO level of the substance with an electron-transport property included in the electron-relay layer 118 is preferably between the LUMO level of the acceptor substance in the p-type layer 117 and the LUMO level of a substance included in a layer of the electron-transport layer 114 that is in contact with the charge-generation layer 116. As a specific value of the energy level, the LUMO level of the substance with an electron-transport property in the electron-relay layer 118 is preferably higher than or equal to −5.0 eV, further preferably higher than or equal to −5.0 eV and lower than or equal to −3.0 eV. Note that as the substance with an electron-transport property in the electron-relay layer 118, a phthalocyanine-based material or a metal complex having a metal-oxygen bond and an aromatic ligand is preferably used.

The electron-injection buffer layer 119 can be formed using a substance having a high electron-injection property, e.g., an alkali metal, an alkaline earth metal, a rare earth metal, or a compound thereof (an alkali metal compound (including an oxide such as lithium oxide, a halide, and a carbonate such as lithium carbonate or cesium carbonate), an alkaline earth metal compound (including an oxide, a halide, and a carbonate), or a rare earth metal compound (including an oxide, a halide, and a carbonate)).

In the case where the electron-injection buffer layer 119 contains a substance with an electron-transport property and a donor substance, an organic compound such as tetrathianaphthacene (abbreviation: TTN), nickelocene, or decamethylnickelocene can be used as the donor substance, as well as an alkali metal, an alkaline earth metal, a rare earth metal, or a compound thereof (e.g., an alkali metal compound (including an oxide such as lithium oxide, a halide, and a carbonate such as lithium carbonate and cesium carbonate), an alkaline earth metal compound (including an oxide, a halide, and a carbonate), or a rare earth metal compound (including an oxide, a halide, and a carbonate)).

As the substance with an electron-transport property, a material similar to the above-described material for the electron-transport layer 114 can be used. Since the above-described material is an organic compound having a low refractive index, the use of the material for the electron-injection buffer layer 119 can offer a light-emitting device with high external quantum efficiency.

As a substance of the cathode 102, any of metals, alloys, and electrically conductive compounds with a low work function (specifically, lower than or equal to 3.8 eV), mixtures thereof, and the like can be used. Specific examples of such a cathode material include elements belonging to Group 1 and Group 2 of the periodic table, such as alkali metals (e.g., lithium (Li) and cesium (Cs)), magnesium (Mg), calcium (Ca), and strontium (Sr), alloys containing these elements (e.g., MgAg and AlLi), rare earth metals such as europium (Eu) and ytterbium (Yb), and alloys containing these rare earth metals. However, when the electron-injection layer is provided between the cathode 102 and the electron-transport layer, any of a variety of conductive materials such as Al, Ag, ITO, or indium oxide-tin oxide containing silicon or silicon oxide can be used for the cathode 102 regardless of the work function.

In the case where the cathode 102 is formed using a material having a visible-light-transmitting property, the light-emitting device can emit light from the cathode side as illustrated in FIG. 1D. In the case where the anode 101 is formed on the substrate side, the light-emitting device including such a cathode can be what is called a top-emission light-emitting device.

Films of these conductive materials can be formed by a dry process such as a vacuum evaporation method or a sputtering method, an ink-jet method, a spin coating method, or the like. Alternatively, a wet process using a sol-gel method or a wet process using a paste of a metal material may be employed.

Any of a variety of methods can be used for forming the EL layer 103, regardless of whether it is a dry process or a wet process. For example, a vacuum evaporation method, a gravure printing method, an offset printing method, a screen printing method, an ink-jet method, a spin coating method, or the like may be used.

Different methods may be used to form the electrodes or the layers described above.

The structure of the layers provided between the anode 101 and the cathode 102 is not limited to the above-described structure. Preferably, a light-emitting region where holes and electrons recombine is positioned away from the anode 101 and the cathode 102 so as to inhibit quenching due to the proximity of the light-emitting region and a metal used for electrodes or carrier-injection layers.

Furthermore, in order that transfer of energy from an exciton generated in the light-emitting layer can be inhibited, the hole-transport layer or the electron-transport layer, which is in contact with the light-emitting layer 113, particularly a carrier-transport layer closer to the recombination region in the light-emitting layer 113, is preferably formed using a substance having a wider band gap than the light-emitting material of the light-emitting layer or the light-emitting material included in the light-emitting layer.

Next, an embodiment of a light-emitting device with a structure in which a plurality of light-emitting units are stacked (also referred to as a stacked element or a tandem element) is described. This light-emitting device includes a plurality of light-emitting units between an anode and a cathode. One light-emitting unit has substantially the same structure as the EL layer 103 illustrated in FIG. 1A. In other words, the light-emitting device illustrated in FIG. 1A or FIG. 1B includes a single light-emitting unit, and the tandem element includes a plurality of light-emitting units.

In the tandem element, a first light-emitting unit and a second light-emitting unit are stacked between an anode and a cathode, and a charge-generation layer is provided between the first light-emitting unit and the second light-emitting unit. The anode and the cathode correspond, respectively, to the anode 101 and the cathode 102 in FIG. 1A, and the same materials as those given in the description for FIG. 1A can be used. The first light-emitting unit and the second light-emitting unit may have the same structure or different structures.

The charge-generation layer in the tandem element has a function of injecting electrons into one of the light-emitting units and injecting holes into the other of the light-emitting units when voltage is applied between the anode and the cathode. That is, the charge-generation layer injects electrons into the first light-emitting unit and holes into the second light-emitting unit when voltage is applied such that the potential of the anode becomes higher than the potential of the cathode.

The charge-generation layer preferably has a structure similar to that of the charge-generation layer 116 described with reference to FIG. 1B. A composite material of an organic compound and a metal oxide has an excellent carrier-injection property and an excellent carrier-transport property; thus, low-voltage driving and low-current driving can be achieved. In the case where the anode-side surface of a light-emitting unit is in contact with the charge-generation layer, the charge-generation layer can also function as a hole-injection layer of the light-emitting unit; therefore, a hole-injection layer is not necessarily provided in the light-emitting unit.

In the case where the charge-generation layer of the tandem element includes the electron-injection buffer layer 119, the electron-injection buffer layer 119 functions as the electron-injection layer in the light-emitting unit on the anode side; thus, an electron-injection layer is not necessarily formed in the light-emitting unit on the anode side.

The tandem element having two light-emitting units is described above; one embodiment of the present invention can also be applied to a tandem element in which three or more light-emitting units are stacked. With a plurality of light-emitting units partitioned by the charge-generation layer between a pair of electrodes, it is possible to provide a long-life element that can emit light with high luminance at a low current density. A light-emitting apparatus that can be driven at a low voltage and has low power consumption can be provided.

When the emission colors of the light-emitting units are different, light emission of a desired color can be obtained from the light-emitting device as a whole. For example, in a light-emitting device having two light-emitting units, the emission colors of the first light-emitting unit may be red and green and the emission color of the second light-emitting unit may be blue, so that the light-emitting device can emit white light as a whole.

The above-described layers or electrodes such as the EL layer 103, the first light-emitting unit, the second light-emitting unit, and the charge-generation layer can be formed by a method such as an evaporation method (including a vacuum evaporation method), a droplet discharge method (also referred to as an ink-jet method), a coating method, or a gravure printing method, for example. A low molecular weight material, a middle molecular weight material (including an oligomer and a dendrimer), or a high molecular weight material may be included in the layers or electrodes.

This embodiment can be freely combined with any of the other embodiments.

### Embodiment 2

In this embodiment, a light-emitting apparatus that includes the light-emitting device described in Embodiment 1 will be described.

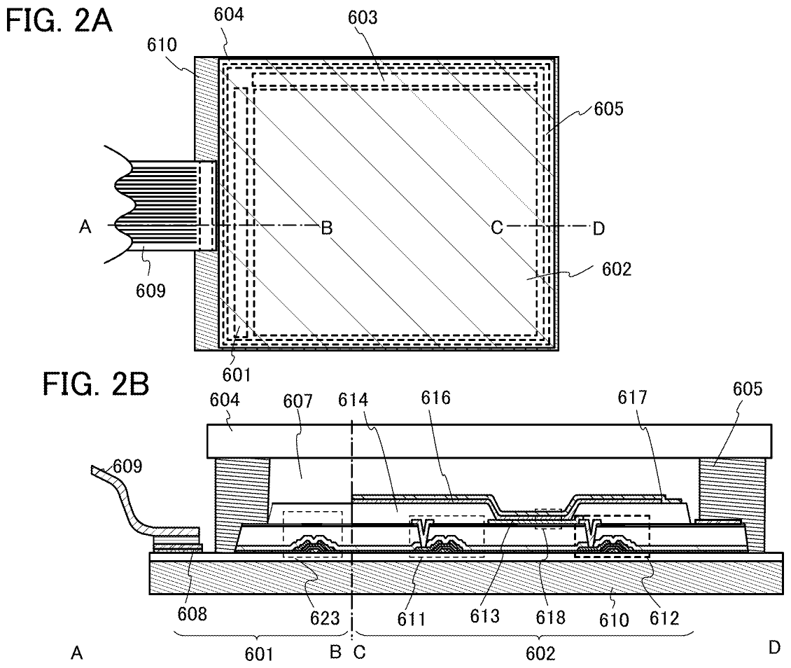

In this embodiment, a light-emitting apparatus manufactured using the light-emitting device described in Embodiment 1 is described with reference to FIG. 2A and FIG. 2B. Note that FIG. 2A is a top view of the light-emitting apparatus and FIG. 2B is a cross-sectional view taken along the dashed-dotted line A-B and the dashed-dotted line C-D in FIG. 2A. This light-emitting apparatus includes a driver circuit portion (source line driver circuit) 601, a pixel portion 602, and a driver circuit portion (gate line driver circuit) 603, which are to control light emission of the light-emitting device and are illustrated with dotted lines. Reference numeral 604 denotes a sealing substrate; 605, a sealing material; and 607, a space surrounded by the sealing material 605.

A lead wiring 608 is a wiring for transmitting signals to be input to the source line driver circuit 601 and the gate line driver circuit 603 and receives a video signal, a clock signal, a start signal, a reset signal, or the like from an FPC (flexible printed circuit) 609 serving as an external input terminal. Although only the FPC is illustrated here, a printed wiring board (PWB) may be attached to the FPC. The light-emitting apparatus in this specification includes, in its category, not only the light-emitting apparatus itself but also the light-emitting apparatus provided with an FPC or a PWB.

Next, a cross-sectional structure is described with reference to FIG. 2B. The driver circuit portions and the pixel portion are formed over an element substrate 610; here, the source line driver circuit 601, which is a driver circuit portion, and one pixel in the pixel portion 602 are illustrated.

The element substrate 610 may be formed using a substrate containing glass, quartz, an organic resin, a metal, an alloy, a semiconductor, or the like or a plastic substrate formed of FRP (Fiber Reinforced Plastics), PVF (poly(vinyl fluoride)), a polyester, an acrylic resin, or the like.

The structure of transistors used in pixels or driver circuits is not particularly limited. For example, inverted staggered transistors may be used, or staggered transistors may be used. Furthermore, top-gate transistors or bottom-gate transistors may be used. A semiconductor material used for the transistors is not particularly limited, and for example, silicon, germanium, silicon carbide, gallium nitride, or the like can be used. Alternatively, an oxide semiconductor containing at least one of indium, gallium, and zinc, such as In—Ga—Zn oxide, may be used.

There is no particular limitation on the crystallinity of a semiconductor material used for the transistors, and an amorphous semiconductor or a semiconductor having crystallinity (a microcrystalline semiconductor, a polycrystalline semiconductor, a single crystal semiconductor, or a semiconductor partly including crystal regions) may be used. A semiconductor having crystallinity is preferably used to inhibit deterioration of the transistor characteristics.

Here, an oxide semiconductor is preferably used for semiconductor devices such as the transistors provided in the pixels or driver circuits and transistors used for touch sensors described later and the like. In particular, an oxide semiconductor having a wider band gap than silicon is preferably used. When an oxide semiconductor having a wider band gap than silicon is used, the off-state current of the transistors can be reduced.

The oxide semiconductor preferably contains at least indium (In) or zinc (Zn). Further preferably, the oxide semiconductor contains an oxide represented by an In-M-Zn oxide (M represents a metal such as Al, Ti, Ga, Ge, Y, Zr, Sn, La, Ce, or Hf).

As a semiconductor layer, it is particularly preferable to use an oxide semiconductor film including a plurality of crystal parts whose c-axes are aligned perpendicular to a surface on which the semiconductor layer is formed or the top surface of the semiconductor layer and in which the adjacent crystal parts have no grain boundary.

The use of such materials for the semiconductor layer makes it possible to provide a highly reliable transistor in which a change in the electrical characteristics is inhibited.

Charge accumulated in a capacitor through a transistor including the above-described semiconductor layer can be held for a long time because of the low off-state current of the transistor. When such a transistor is used in a pixel, operation of a driver circuit can be stopped while a gray scale of an image displayed in each display region is maintained. As a result, a light-emitting apparatus with extremely low power consumption can be obtained.

For stable characteristics or the like of the transistor, a base film is preferably provided. The base film can be formed with a single layer or stacked layers using an inorganic insulating film such as a silicon oxide film, a silicon nitride film, a silicon oxynitride film, or a silicon nitride oxide film. The base film can be formed by a sputtering method, a chemical vapor deposition (CVD) method (e.g., a plasma CVD method, a thermal CVD method, or an MOCVD (Metal Organic CVD) method), an atomic layer deposition (ALD) method, a coating method, a printing method, or the like. Note that the base film is not necessarily provided.

Note that an FET 623 is illustrated as a transistor formed in the driver circuit portion 601. The driver circuit may be formed with any of a variety of circuits such as a CMOS circuit, a PMOS circuit, or an NMOS circuit. Although a driver integrated type in which the driver circuit is formed over the substrate is described in this embodiment, the driver circuit is not necessarily formed over the substrate, and the driver circuit can be formed outside, not over the substrate.

The pixel portion 602 includes a plurality of pixels each including a switching FET 611, a current controlling FET 612, and an anode 613 electrically connected to a drain of the current controlling FET 612. One embodiment of the present invention is not limited to the structure, and a pixel portion in which three or more FETs and a capacitor are combined may be employed.

Note that an insulator 614 is formed to cover an end portion of the anode 613. Here, the insulator 614 can be formed using a positive photosensitive acrylic resin film.

In order to improve the coverage with an EL layer or the like which is formed later, the insulator 614 is formed to have a curved surface with curvature at its upper or lower end portion. For example, in the case where a positive photosensitive acrylic resin is used as a material of the insulator 614, only the upper end portion of the insulator 614 preferably has a curved surface with a curvature radius (0.2 μm to 3 μm). As the insulator 614, either a negative photosensitive resin or a positive photosensitive resin can be used.

An EL layer 616 and a cathode 617 are formed over the anode 613. Here, a material having a high work function is preferably used as a material of the anode 613. For example, a single-layer film of an ITO film, an indium tin oxide film containing silicon, an indium oxide film containing zinc oxide at 2 wt % to 20 wt %, a titanium nitride film, a chromium film, a tungsten film, a Zn film, a Pt film, or the like, a stack of a titanium nitride film and a film containing aluminum as its main component, a stack of three layers of a titanium nitride film, a film containing aluminum as its main component, and a titanium nitride film, or the like can be used. The stacked-layer structure enables low wiring resistance, favorable ohmic contact, and a function as an anode.

The EL layer 616 is formed by any of a variety of methods such as an evaporation method using an evaporation mask, an inkjet method, and a spin coating method. The EL layer 616 has the structure described in Embodiment 1. As another material included in the EL layer 616, a low molecular weight compound or a high molecular weight compound (including an oligomer or a dendrimer) may be used.

As a material used for the cathode 617, which is formed over the EL layer 616, a material having a low work function (e.g., Al, Mg, Li, and Ca, or an alloy or a compound thereof, such as MgAg, MgIn, and AlLi) is preferably used. In the case where light generated in the EL layer 616 passes through the cathode 617, a stack of a thin metal film and a transparent conductive film (e.g., ITO, indium oxide containing zinc oxide at 2 wt % to 20 wt %, an indium tin oxide containing silicon, or zinc oxide (ZnO)) is preferably used for the cathode 617.

Note that the light-emitting device is formed with the anode 613, the EL layer 616, and the cathode 617. The light-emitting device is the light-emitting device described in Embodiment 1. In the light-emitting apparatus of this embodiment, the pixel portion, which includes a plurality of light-emitting devices, may include both the light-emitting device described in Embodiment 1 and a light-emitting device having a different structure.

The sealing substrate 604 is attached to the element substrate 610 with the sealing material 605, so that a light-emitting device 618 is provided in the space 607 surrounded by the element substrate 610, the sealing substrate 604, and the sealing material 605. The space 607 is filled with a filler, or may be filled with an inert gas (such as nitrogen or argon) or the sealing material. It is preferable that the sealing substrate be provided with a recessed portion and a drying agent be provided in the recessed portion, in which case deterioration due to influence of moisture can be inhibited.

An epoxy-based resin or glass frit is preferably used for the sealing material 605. It is preferable that such a material transmit moisture or oxygen as little as possible. As the sealing substrate 604, a glass substrate, a quartz substrate, or a plastic substrate formed of FRP (Fiber Reinforced Plastics), PVF (poly(vinyl fluoride)), a polyester, an acrylic resin, or the like can be used.

Although not illustrated in FIG. 2A and FIG. 2B, a protective film may be provided over the cathode. As the protective film, an organic resin film or an inorganic insulating film may be formed. The protective film may be formed so as to cover an exposed portion of the sealing material 605. The protective film may be provided so as to cover surfaces and side surfaces of the pair of substrates and exposed side surfaces of a sealing layer, an insulating layer, and the like.

The protective film can be formed using a material that does not easily transmit an impurity such as water. Thus, diffusion of an impurity such as water from the outside into the inside can be effectively inhibited.

As a material of the protective film, an oxide, a nitride, a fluoride, a sulfide, a ternary compound, a metal, a polymer, or the like can be used. For example, a material containing aluminum oxide, hafnium oxide, hafnium silicate, lanthanum oxide, silicon oxide, strontium titanate, tantalum oxide, titanium oxide, zinc oxide, niobium oxide, zirconium oxide, tin oxide, yttrium oxide, cerium oxide, scandium oxide, erbium oxide, vanadium oxide, indium oxide, or the like; a material containing aluminum nitride, hafnium nitride, silicon nitride, tantalum nitride, titanium nitride, niobium nitride, molybdenum nitride, zirconium nitride, gallium nitride, or the like; a material containing a nitride containing titanium and aluminum, an oxide containing titanium and aluminum, an oxide containing aluminum and zinc, a sulfide containing manganese and zinc, a sulfide containing cerium and strontium, an oxide containing erbium and aluminum, an oxide containing yttrium and zirconium, or the like can be used.

The protective film is preferably formed using a deposition method that enables favorable step coverage. One such method is an ALD method. A material that can be deposited by an ALD method is preferably used for the protective film. A dense protective film having reduced defects such as cracks or pinholes or a uniform thickness can be formed by an ALD method. Furthermore, damage caused to a process member in forming the protective film can be reduced.

By an ALD method, a uniform protective film with few defects can be formed even on, for example, a surface with a complex uneven shape or upper, side, and lower surfaces of a touch panel.

As described above, the light-emitting apparatus manufactured using the light-emitting device described in Embodiment 1 can be obtained.

The light-emitting apparatus in this embodiment is manufactured using the light-emitting device described in Embodiment 1 and thus can have favorable characteristics. Specifically, since the light-emitting device described in Embodiment 1 has favorable emission efficiency, the light-emitting apparatus can achieve low power consumption.

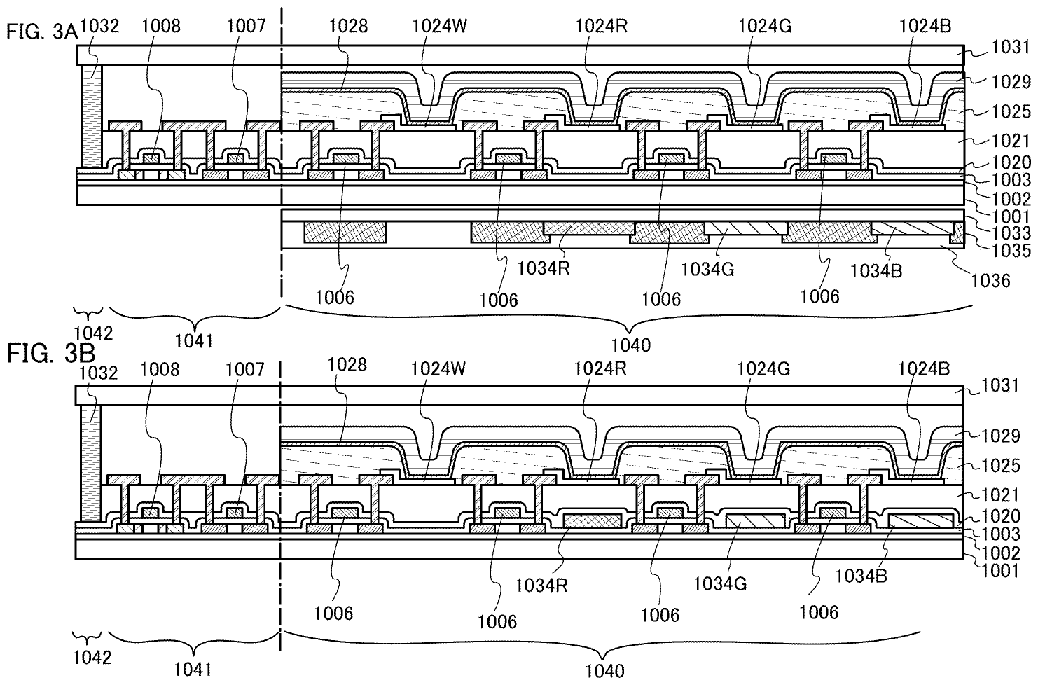

FIG. 3A and FIG. 3B each illustrate an example of a light-emitting apparatus that includes a light-emitting device exhibiting white light emission and coloring layers (color filters) and the like to display a full-color image. FIG. 3A illustrates a substrate 1001, a base insulating film 1002, a gate insulating film 1003, a gate electrode 1006, a gate electrode 1007, a gate electrode 1008, a first interlayer insulating film 1020, a second interlayer insulating film 1021, a peripheral portion 1042, a pixel portion 1040, a driver circuit portion 1041, an anode 1024W of the light-emitting device, an anode 1024R of the light-emitting device, an anode 1024G of the light-emitting device, an anode 1024B of the light-emitting device, a partition 1025, an EL layer 1028, a cathode 1029 of the light-emitting devices, a sealing substrate 1031, a sealing material 1032, and the like.

In FIG. 3A, coloring layers (a red coloring layer 1034R, a green coloring layer 1034G, and a blue coloring layer 1034B) are provided on a transparent base material 1033. A black matrix 1035 may be additionally provided. The transparent base material 1033 provided with the coloring layers and the black matrix 1035 is aligned and fixed to the substrate 1001. Note that the coloring layers and the black matrix 1035 are covered with an overcoat layer. In FIG. 3A, light emitted from part of the light-emitting layer does not pass through the coloring layers and is released to the outside, while light emitted from the other part of the light-emitting layer passes through the coloring layers and is released to the outside. The light that does not pass through the coloring layers is white and the light that passes through any one of the coloring layers is red, green, or blue; thus, an image can be expressed using pixels of the four colors.

FIG. 3B shows an example in which the coloring layers (the red coloring layer 1034R, the green coloring layer 1034G, and the blue coloring layer 1034B) are provided between the gate insulating film 1003 and the first interlayer insulating film 1020. As in the structure, the coloring layers may be provided between the substrate 1001 and the sealing substrate 1031.

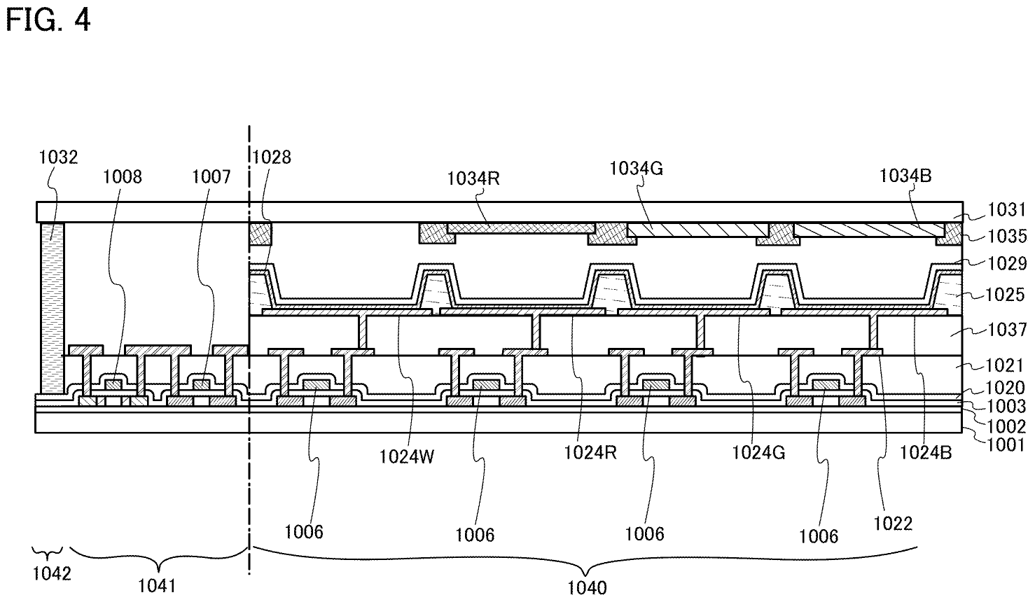

The above-described light-emitting apparatus has a structure in which light is extracted from the substrate 1001 side where FETs are formed (a bottom emission structure), but may have a structure in which light is extracted from the sealing substrate 1031 side (atop emission structure). FIG. 4 is a cross-sectional view of a light-emitting apparatus having a top emission structure. In this case, a substrate that does not transmit light can be used as the substrate 1001. The process up to the step of forming a connection electrode which connects the FET and the anode of the light-emitting device is performed in a manner similar to that of the light-emitting apparatus having a bottom emission structure. Then, a third interlayer insulating film 1037 is formed to cover the electrode 1022. This insulating film may have a planarization function. The third interlayer insulating film 1037 can be formed using a material similar to that of the second interlayer insulating film, and can alternatively be formed using any of other known materials.

The anode 1024W of the light-emitting device, the anode 1024R of the light-emitting device, the anode 1024G of the light-emitting device, and the anode 1024B of the light-emitting device each serve as an anode here, but may each serve as a cathode. Furthermore, in the case of a light-emitting apparatus having a top emission structure as illustrated in FIG. 4, the anodes are preferably reflective electrodes. The EL layer 1028 is formed to have a structure similar to the structure of the EL layer 103 described in Embodiment 1, with which white light emission can be obtained.

In the case of a top emission structure as illustrated in FIG. 4, sealing can be performed with the sealing substrate 1031 on which the coloring layers (the red coloring layer 1034R, the green coloring layer 1034G, and the blue coloring layer 1034B) are provided. The sealing substrate 1031 may be provided with the black matrix 1035 which is positioned between pixels. The coloring layers (the red coloring layer 1034R, the green coloring layer 1034G, and the blue coloring layer 1034B) and the black matrix 1035 may be covered with the overcoat layer. Note that a light-transmitting substrate is used as the sealing substrate 1031. Although an example in which full color display is performed using four colors of red, green, blue, and white is shown here, there is no particular limitation and full color display using four colors of red, yellow, green, and blue or three colors of red, green, and blue may be performed.

In the light-emitting apparatus having a top emission structure, a microcavity structure can be favorably employed. A light-emitting device with a microcavity structure is formed with the use of a reflective electrode as the anode and a transflective electrode as the cathode. The light-emitting device with a microcavity structure includes at least an EL layer between the reflective electrode and the transflective electrode, and the EL layer includes at least a light-emitting layer serving as a light-emitting region.

Note that the reflective electrode has a visible light reflectivity of 40% to 100%, preferably 70% to 100%, and a resistivity of 1×10⁻² Ωcm or lower. In addition, the transflective electrode is a film that has a visible light reflectivity of 20% to 80%, preferably 40% to 70%, and a resistivity of 1×10⁻² Ωcm or lower.

Light emitted from the light-emitting layer included in the EL layer is reflected and resonated by the reflective electrode and the transflective electrode.

In the light-emitting device, by changing the thicknesses of the transparent conductive film, the composite material, the carrier-transport material, or the like, the optical path length between the reflective electrode and the transflective electrode can be changed. Thus, light with a wavelength that is resonated between the reflective electrode and the transflective electrode can be intensified while light with a wavelength that is not resonated therebetween can be attenuated.

Note that light that is reflected back by the reflective electrode (first reflected light) considerably interferes with light that directly enters the transflective electrode from the light-emitting layer (first incident light). For this reason, the optical path length between the reflective electrode and the light-emitting layer is preferably adjusted to (2n−1)λ/4 (n is a natural number of 1 or more and λ is the wavelength of light to be amplified). By adjusting the optical path length, the phases of the first reflected light and the first incident light can be aligned with each other and the light emitted from the light-emitting layer can be further amplified.

Note that in the above structure, the EL layer may include a plurality of light-emitting layers or may include a single light-emitting layer; for example, in combination with the structure of the above-described tandem light-emitting device, a plurality of EL layers each including a single or a plurality of light-emitting layer(s) may be provided in one light-emitting device with a charge-generation layer interposed between the EL layers.

With the microcavity structure, emission intensity with a specific wavelength in the front direction can be increased, whereby power consumption can be reduced. Note that in the case of a light-emitting apparatus that displays images with subpixels of four colors, red, yellow, green, and blue, the light-emitting apparatus can have favorable characteristics because the luminance can be increased owing to yellow light emission and each subpixel can employ a microcavity structure suitable for the wavelength of the corresponding color.

The light-emitting apparatus in this embodiment is manufactured using the light-emitting device described in Embodiment 1 and thus can have favorable characteristics. Specifically, since the light-emitting device described in Embodiment 1 has favorable emission efficiency, the light-emitting apparatus can achieve low power consumption.

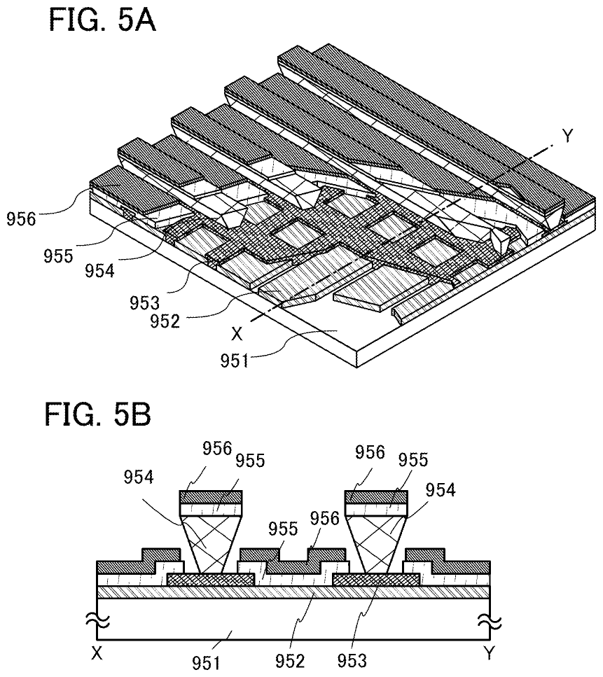

The active matrix light-emitting apparatus is described above, whereas a passive matrix light-emitting apparatus is described below. FIG. 5A and FIG. 5B illustrate a passive matrix light-emitting apparatus manufactured using the present invention. Note that FIG. 5A is a perspective view of the light-emitting apparatus, and FIG. 5B is a cross-sectional view taken along the dashed-dotted line X-Y in FIG. 5A. In FIG. 5, over a substrate 951, an EL layer 955 is provided between an electrode 952 and an electrode 956. An end portion of the electrode 952 is covered with an insulating layer 953. A partition layer 954 is provided over the insulating layer 953. The sidewalls of the partition layer 954 are aslope such that the distance between both sidewalls is gradually narrowed toward the surface of the substrate. In other words, a cross section taken along the direction of the short side of the partition layer 954 is trapezoidal, and the lower side (a side that is parallel to the surface of the insulating layer 953 and is in contact with the insulating layer 953) is shorter than the upper side (a side that is parallel to the surface of the insulating layer 953 and is not in contact with the insulating layer 953). The partition layer 954 thus provided can prevent defects in the light-emitting device due to static electricity or the like. The passive matrix light-emitting apparatus also includes the light-emitting device described in Embodiment 1; thus, the light-emitting apparatus can have favorable reliability or low power consumption.

In the light-emitting apparatus described above, many minute light-emitting devices arranged in a matrix can each be controlled; thus, the light-emitting apparatus can be suitably used as a display device for displaying images.

This embodiment can be freely combined with any of the other embodiments.

### Embodiment 3

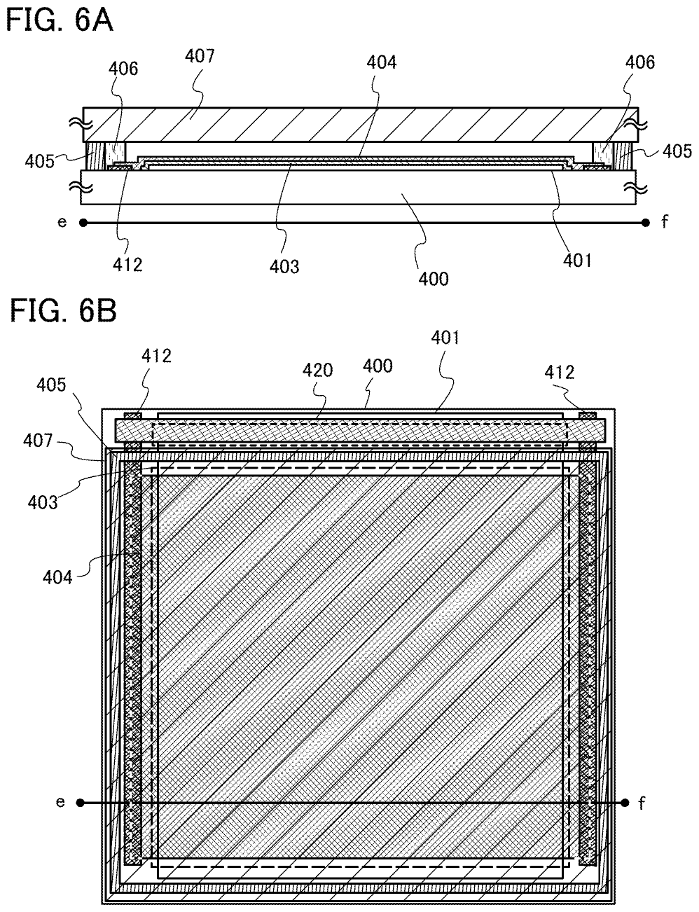

In this embodiment, an example in which the light-emitting device described in Embodiment 1 is used for a lighting device will be described with reference to FIG. 6. FIG. 6B is a top view of the lighting device, and FIG. 6A is a cross-sectional view taken along the line e-f in FIG. 6B.

In the lighting device in this embodiment, an anode 401 is formed over a substrate 400 which is a support with a light-transmitting property. The anode 401 corresponds to the anode 101 in Embodiment 1. When light is extracted through the anode 401, the anode 401 is formed using a material having a light-transmitting property.

A pad 412 for applying voltage to a cathode 404 is provided over the substrate 400.

An EL layer 403 is formed over the anode 401. The structure of the EL layer 403 corresponds to, for example, the structure of the EL layer 103 in Embodiment 1, or the structure in which the first light-emitting unit, the second light-emitting unit, and a charge-generation layer are combined. Note that for these structures, the corresponding description can be referred to.

The cathode 404 is formed to cover the EL layer 403. The cathode 404 corresponds to the cathode 102 in Embodiment 1. The cathode 404 is formed using a material having high reflectance when light is extracted through the anode 401. The cathode 404 is connected to the pad 412, thereby receiving voltage.

As described above, the lighting device described in this embodiment includes a light-emitting device including the anode 401, the EL layer 403, and the cathode 404. Since the light-emitting device is a light-emitting device with high emission efficiency, the lighting device in this embodiment can have low power consumption.

The substrate 400 provided with the light-emitting device having the above structure is fixed to a sealing substrate 407 with a sealing material 405 and a sealing material 406 and sealing is performed, whereby the lighting device is completed. It is possible to use only either the sealing material 405 or the sealing material 406. The inner sealing material 406 (not illustrated in FIG. 6B) can be mixed with a desiccant which enables moisture to be adsorbed, increasing reliability.

When parts of the pad 412 and the anode 401 are extended to the outside of the sealing material 405 and the sealing material 406, the extended parts can serve as external input terminals. An IC chip 420 mounted with a converter or the like may be provided over the external input terminals, for example.

The lighting device described in this embodiment includes as an EL element the light-emitting device described in Embodiment 1; thus, the lighting device can have low power consumption.

This embodiment can be freely combined with any of the other embodiments.

### Embodiment 4

In this embodiment, examples of electronic apparatuses each including the light-emitting device described in Embodiment 1 will be described. The light-emitting device described in Embodiment 1 has favorable emission efficiency and low power consumption. As a result, the electronic apparatuses described in this embodiment can be electronic apparatuses each including a light-emitting portion with low power consumption.

Examples of the electronic apparatus including the above light-emitting device include television devices (also referred to as TV or television receivers), monitors for computers and the like, digital cameras, digital video cameras, digital photo frames, cellular phones (also referred to as mobile phones or mobile phone devices), portable game machines, portable information terminals, audio playback devices, and large game machines such as pachinko machines. Specific examples of these electronic apparatuses are described below.

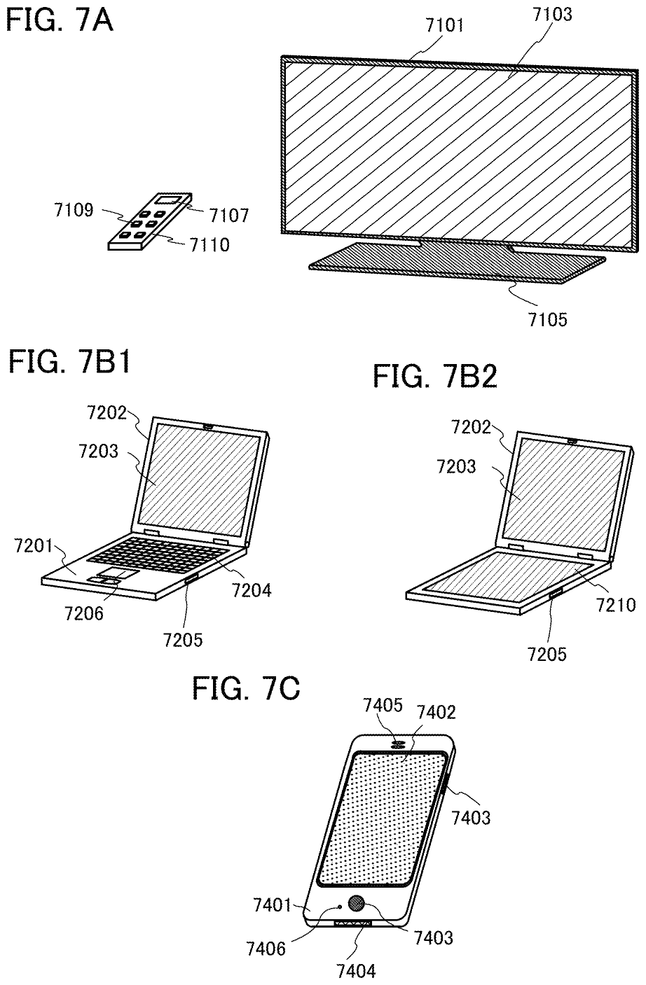

FIG. 7A shows an example of a television device. In the television device, a display portion 7103 is incorporated in a housing 7101. Here, the housing 7101 is supported by a stand 7105. Images can be displayed on the display portion 7103, and in the display portion 7103, the light-emitting devices described in Embodiment 1 are arranged in a matrix.

The television device can be operated with an operation switch of the housing 7101 and a separate remote controller 7110. With operation keys 7109 of the remote controller 7110, channels and volume can be controlled and images displayed on the display portion 7103 can be controlled. Furthermore, the remote controller 7110 may be provided with a display portion 7107 for displaying data output from the remote controller 7110. The light-emitting devices described in Embodiment 1 may also be arranged in a matrix in the display portion 7107.

Note that the television device is provided with a receiver, a modem, and the like. With the use of the receiver, a general television broadcast can be received, and moreover, when the television device is connected to a communication network with or without wires via the modem, one-way (from a sender to a receiver) or two-way (between a sender and a receiver or between receivers) data communication can be performed.

FIG. 7B1 illustrates a computer, which includes a main body 7201, a housing 7202, a display portion 7203, a keyboard 7204, an external connection port 7205, a pointing device 7206, and the like. Note that this computer is manufactured by arranging the light-emitting devices described in Embodiment 1 in a matrix in the display portion 7203. The computer illustrated in FIG. 7B1 may have a structure illustrated in FIG. 7B2. A computer illustrated in FIG. 7B2 is provided with a display portion 7210 instead of the keyboard 7204 and the pointing device 7206. The display portion 7210 is a touch panel, and input operation can be performed by touching display for input on the display portion 7210 with a finger or a dedicated pen. The display portion 7210 can also display images other than the display for input. The display portion 7203 may also be a touch panel. Connecting the two screens with a hinge can prevent troubles; for example, the screens can be prevented from being cracked or broken while the computer is being stored and carried.

FIG. 7C shows an example of a portable terminal. A cellular phone is provided with a display portion 7402 incorporated in a housing 7401, operation buttons 7403, an external connection port 7404, a speaker 7405, a microphone 7406, and the like. Note that the cellular phone has the display portion 7402 in which the light-emitting devices described in Embodiment 1 are arranged in a matrix.

When the display portion 7402 of the portable terminal illustrated in FIG. 7C is touched with a finger or the like, data can be input to the portable terminal. In this case, operations such as making a call and creating an e-mail can be performed by touching the display portion 7402 with a finger or the like.

The display portion 7402 has mainly three screen modes. The first mode is a display mode mainly for displaying images. The second mode is an input mode mainly for inputting data such as text. The third mode is a display-and-input mode in which the two modes, the display mode and the input mode, are combined.

For example, in the case of making a call or creating an e-mail, a text input mode mainly for inputting text is selected for the display portion 7402 so that text displayed on the screen can be input. In this case, it is preferable to display a keyboard or number buttons on almost the entire screen of the display portion 7402.

When a detection device including a sensor such as a gyroscope sensor or an acceleration sensor for detecting inclination is provided inside the portable terminal, display on the screen of the display portion 7402 can be automatically changed by determining the orientation of the portable terminal (whether the portable terminal is placed horizontally or vertically).

The screen modes are switched by touching the display portion 7402 or operating the operation buttons 7403 of the housing 7401. Alternatively, the screen modes can be switched depending on the kind of images displayed on the display portion 7402. For example, when a signal of an image displayed on the display portion is a signal of moving image data, the screen mode is switched to the display mode. When the signal is a signal of text data, the screen mode is switched to the input mode.

Moreover, in the input mode, when input by touching the display portion 7402 is not performed for a certain period while a signal detected by an optical sensor in the display portion 7402 is sensed, the screen mode may be controlled so as to be switched from the input mode to the display mode.

The display portion 7402 may also function as an image sensor. For example, an image of a palm print, a fingerprint, or the like is taken when the display portion 7402 is touched with the palm or the finger, whereby personal authentication can be performed. Furthermore, by providing a backlight or a sensing light source which emits near-infrared light in the display portion, an image of a finger vein, a palm vein, or the like can be taken.

Note that the structure described in this embodiment can be combined with any of the structures described in Embodiment 1 to Embodiment 4 as appropriate.

As described above, the application range of the light-emitting apparatus including the light-emitting device described in Embodiment 1 is so wide that this light-emitting apparatus can be used in electronic apparatuses in a variety of fields. By using the light-emitting device described in Embodiment 1, an electronic apparatus with low power consumption can be obtained.

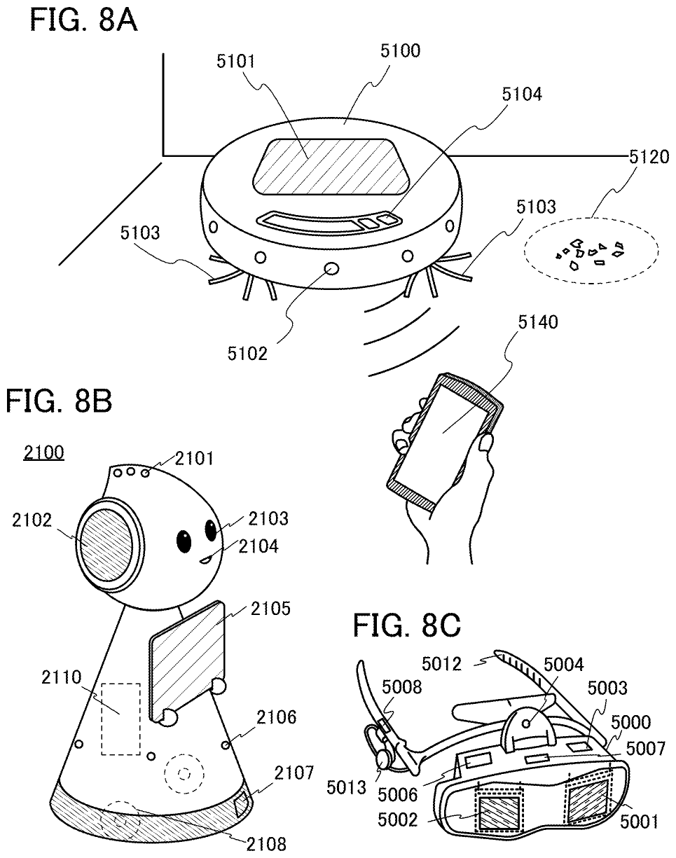

FIG. 8A is a schematic view showing an example of a cleaning robot.

A cleaning robot 5100 includes a display 5101 on its top surface, a plurality of cameras 5102 on its side surface, a brush 5103, and operation buttons 5104. Although not illustrated, the bottom surface of the cleaning robot 5100 is provided with a tire, an inlet, and the like. The cleaning robot 5100 includes various sensors such as an infrared sensor, an ultrasonic sensor, an acceleration sensor, a piezoelectric sensor, an optical sensor, and a gyroscope sensor. The cleaning robot 5100 has a wireless communication means.

The cleaning robot 5100 is self-propelled, senses dust 5120, and vacuums the dust through the inlet provided on the bottom surface.

The cleaning robot 5100 can determine whether there is an obstacle such as a wall, furniture, or a step by analyzing images taken by the cameras 5102. When an object that is likely to be caught in the brush 5103, such as a wire, is sensed by image analysis, the rotation of the brush 5103 can be stopped.

The display 5101 can display the remaining capacity of a battery, the amount of vacuumed dust, and the like. The display 5101 may display a path on which the cleaning robot 5100 has run. The display 5101 may be a touch panel, and the operation buttons 5104 may be provided on the display 5101.

The cleaning robot 5100 can communicate with a portable electronic apparatus 5140 such as a smartphone. Images taken by the cameras 5102 can be displayed on the portable electronic apparatus 5140. Accordingly, an owner of the cleaning robot 5100 can monitor his/her room even when he/she is not at home. The owner can also check the display on the display 5101 by the portable electronic apparatus such as a smartphone.

The light-emitting apparatus of one embodiment of the present invention can be used for the display 5101.

A robot 2100 illustrated in FIG. 8B includes an arithmetic device 2110, an illuminance sensor 2101, a microphone 2102, an upper camera 2103, a speaker 2104, a display 2105, a lower camera 2106, an obstacle sensor 2107, and a moving mechanism 2108.

The microphone 2102 has a function of sensing a speaking voice of a user, an environmental sound, and the like. The speaker 2104 has a function of outputting sound. The robot 2100 can communicate with a user using the microphone 2102 and the speaker 2104.

The display 2105 has a function of displaying various kinds of information. The robot 2100 can display information desired by a user on the display 2105. The display 2105 may be provided with a touch panel. Moreover, the display 2105 may be a detachable information terminal, in which case charging and data communication can be performed when the display 2105 is set at the home position of the robot 2100.

The upper camera 2103 and the lower camera 2106 each have a function of taking an image of the surroundings of the robot 2100. The obstacle sensor 2107 can detect an obstacle in the direction where the robot 2100 advances with the moving mechanism 2108. The robot 2100 can move safely by recognizing the surroundings with the upper camera 2103, the lower camera 2106, and the obstacle sensor 2107. The light-emitting apparatus of one embodiment of the present invention can be used for the display 2105.

FIG. 8C shows an example of a goggle-type display. The goggle-type display includes, for example, a housing 5000, a display portion 5001, a display portion 5002, a speaker 5003, an LED lamp 5004, operation keys (including a power switch or an operation switch), a connection terminal 5006, a sensor 5007 (a sensor having a function of measuring force, displacement, position, speed, acceleration, angular velocity, rotational frequency, distance, light, liquid, magnetism, temperature, chemical substance, sound, time, hardness, electric field, current, voltage, electric power, radiation, flow rate, humidity, gradient, oscillation, odor, or infrared rays), a microphone 5008, a support portion 5012, and an earphone 5013.

The light-emitting apparatus of one embodiment of the present invention can be used for the display portion 5001 and the display portion 5002.

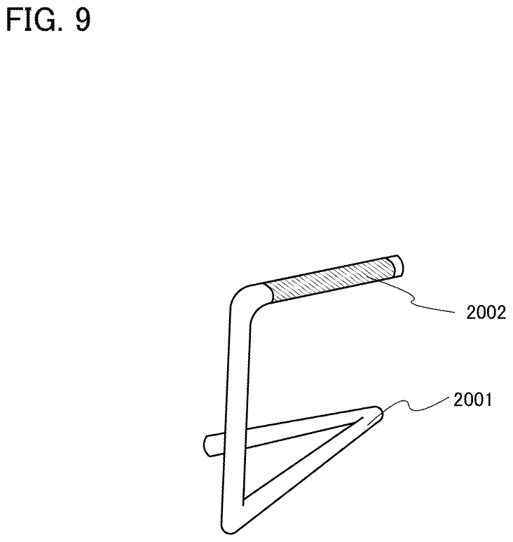

FIG. 9 shows an example in which the light-emitting device described in Embodiment 1 is used for a table lamp which is a lighting device. The table lamp illustrated in FIG. 9 includes a housing 2001 and a light source 2002, and the lighting device described in Embodiment 3 may be used for the light source 2002.

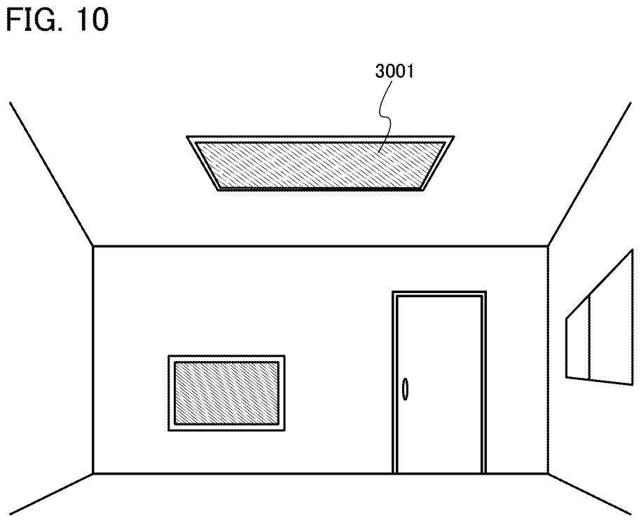

FIG. 10 shows an example in which the light-emitting device described in Embodiment 1 is used for an indoor lighting device 3001. Since the light-emitting device described in Embodiment 1 has high emission efficiency, the lighting device can have low power consumption. Furthermore, since the light-emitting device described in Embodiment 1 can have a large area, the light-emitting device can be used for a large-area lighting device. Furthermore, since the light-emitting device described in Embodiment 1 is thin, the light-emitting device can be used for a lighting device having a reduced thickness.

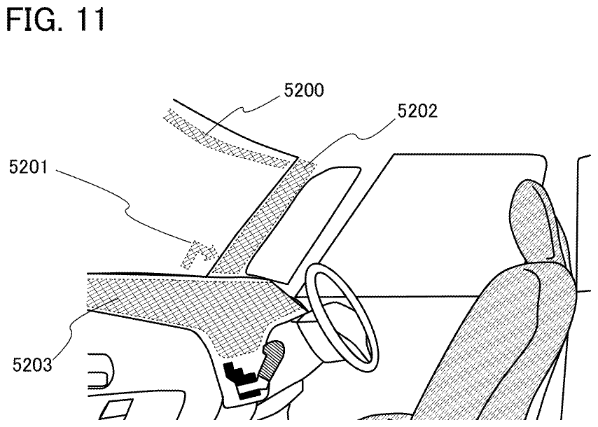

The light-emitting device described in Embodiment 1 can also be used for an automobile windshield and an automobile dashboard. FIG. 11 illustrates a mode in which the light-emitting device described in Embodiment 1 is used for an automobile windshield and an automobile dashboard. A display region 5200 to a display region 5203 each include the light-emitting device described in Embodiment 1.

The display region 5200 and the display region 5201 are display devices which are provided in the automobile windshield and include the light-emitting device described in Embodiment 1. The light-emitting device described in Embodiment 1 can be formed into what is called a see-through display device, through which the opposite side can be seen, by including an anode and a cathode formed of light-transmitting electrodes. Such see-through display devices can be provided even in the automobile windshield without hindering the view. In the case where a driving transistor or the like is provided, a transistor having a light-transmitting property, such as an organic transistor including an organic semiconductor material or a transistor including an oxide semiconductor, is preferably used.

The display region 5202 is a display device which is provided in a pillar portion and includes the light-emitting device described in Embodiment 1. The display region 5202 can compensate for the view hindered by the pillar by displaying an image taken by an imaging means provided in the car body. Similarly, the display region 5203 provided in the dashboard portion can compensate for the view hindered by the car body by displaying an image taken by an imaging means provided on the outside of the automobile; thus, blind areas can be eliminated to enhance the safety. Images that compensate for the areas that a driver cannot see enable the driver to ensure safety easily and comfortably.

The display region 5203 can provide a variety of kinds of information by displaying navigation data, a speedometer, a tachometer, air-condition setting, and the like. The content and layout of the display can be changed as appropriate according to the user's preference. Note that such information can also be displayed on the display region 5200 to the display region 5202. The display region 5200 to the display region 5203 can also be used as lighting devices.

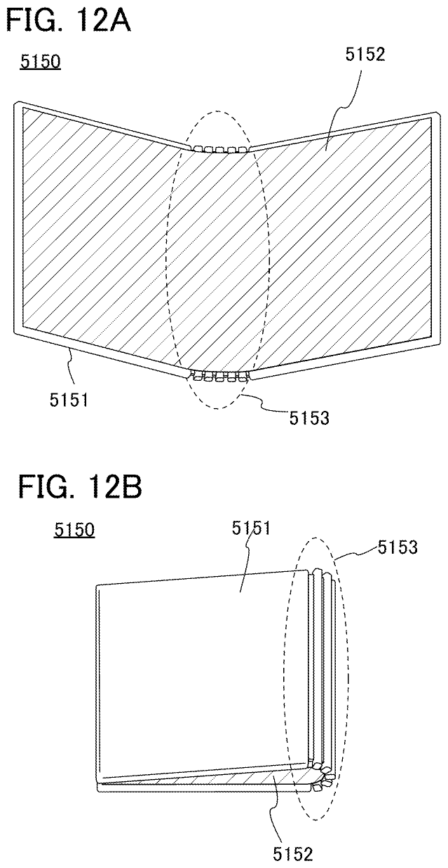

FIG. 12A and FIG. 12B illustrate a foldable portable information terminal 5150. The foldable portable information terminal 5150 includes a housing 5151, a display region 5152, and a bend portion 5153. FIG. 12A illustrates the portable information terminal 5150 that is opened. FIG. 12B illustrates the portable information terminal that is folded. Despite its large display region 5152, the portable information terminal 5150 is compact in size and has excellent portability when folded.

The display region 5152 can be folded in half with the bend portion 5153. The bend portion 5153 includes a flexible member and a plurality of supporting members. When the display region is folded, the flexible member expands and the bend portion 5153 has a radius of curvature of greater than or equal to 2 mm, preferably greater than or equal to 3 mm.

Note that the display region 5152 may be a touch panel (an input/output device) including a touch sensor (an input device). The light-emitting apparatus of one embodiment of the present invention can be used for the display region 5152.

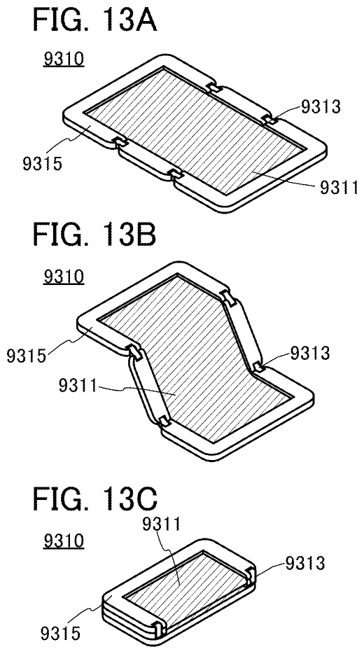

FIG. 13A to FIG. 13C illustrate a foldable portable information terminal 9310. FIG. 13A illustrates the portable information terminal 9310 that is opened. FIG. 13B illustrates the portable information terminal 9310 on the way from either the opened state or the folded state to the other state. FIG. 13C illustrates the portable information terminal 9310 that is folded. The portable information terminal 9310 is excellent in portability when folded, and is excellent in display browsability when opened because of a seamless large display region.

A display panel 9311 is supported by three housings 9315 joined together by hinges 9313. Note that the display panel 9311 may be a touch panel (an input/output device) including a touch sensor (an input device). By folding the display panel 9311 at the hinges 9313 between two of the housings 9315, the portable information terminal 9310 can be reversibly changed in shape from the opened state to the folded state. The light-emitting apparatus of one embodiment of the present invention can be used for the display panel 9311.

### Example 1

### Reference Synthesis Example 1

Described in this example is a method for synthesizing 6-methyl-8-quinolinolato-lithium (abbreviation: Li-6mq) described in Embodiment 1. The structural formula of Li-6mq is shown below.

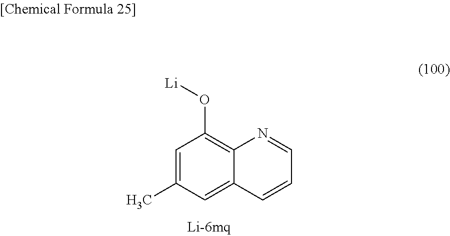

Into a three-neck flask were put 2.0 g (12.6 mmol) of 8-hydroxy-6-methylquinoline and 130 mL of dehydrated tetrahydrofuran (abbreviation: THF), and the mixture was stirred. To this solution was added 10.1 mL (10.1 mmol) of a 1M THF solution of lithium tert-butoxide (abbreviation: tBuOLi), and the mixture was stirred at room temperature for 47 hours. The reacted solution was concentrated to give a yellow solid. Acetonitrile was added to this solid and ultrasonic irradiation and filtration were performed, so that a pale yellow solid was obtained. This washing step was performed twice. The obtained residue was 1.6 g of a pale yellow solid of Li-6mq (yield: 95%). This synthesis scheme is shown below.

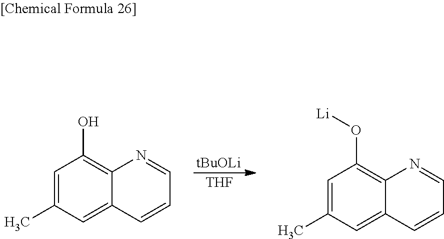

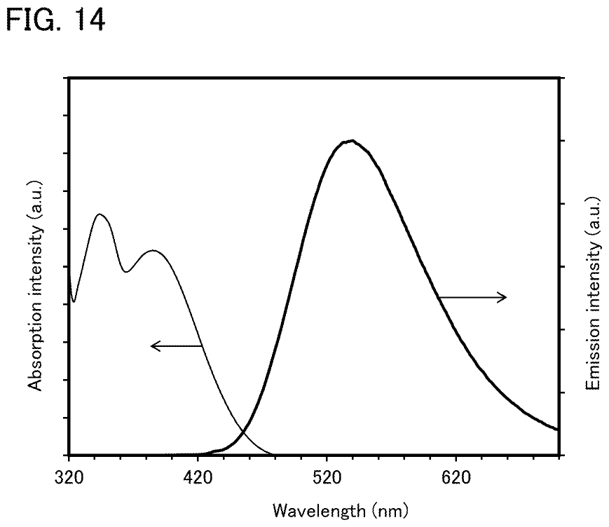

Next, FIG. 14 shows the results of measuring the absorption spectrum and emission spectrum of Li-6mq in a dehydrated acetone solution. The absorption spectrum was measured with an ultraviolet-visible spectrophotometer (V550, manufactured by JASCO Corporation), and the spectrum of dehydrated acetone alone in a quartz cell was subtracted. The emission spectrum was measured with a fluorescence spectrophotometer (FP-8600, produced by JASCO Corporation).

As shown in FIG. 14, Li-6mq in the dehydrated acetone solution had an absorption peak at 390 nm and an emission wavelength peak at 540 nm (excitation wavelength: 385 nm).

### Reference Synthesis Example 2

An example of a synthesis method of the material with a low refractive index and an electron-transport property described in Embodiment 1 is described below.

First, a synthesis method of 2-{(3′,5′-di-tert-butyl)-1,1′-biphenyl-3-yl}-4,6-bis(3,5-di-tert-butylphenyl)-1,3,5-triazine (abbreviation: mmtBumBP-dmmtBuPTzn), which is an organic compound represented by Structural Formula (200), is described. The structure of mmtBumBP-dmmtBuPTzn is shown below.

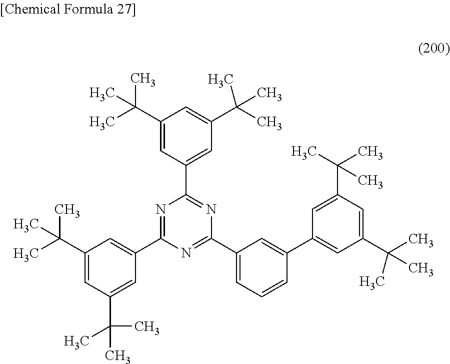 [SMILES: CC(C)(C)c1cc(-c2cccc(-c3nc(-c4cc(C(C)(C)C)cc(C(C)(C)C)c4)nc(-c4cc(C(C)(C)C)cc(C(C)(C)C)c4)n3)c2)cc(C(C)(C)C)c1]

### Step 1: Synthesis of 3-bromo-3′,5′-di-tert-butylbiphenyl

Into a three-neck flask were put 1.0 g (4.3 mmol) of 3,5-di-tert-butylphenylboronic acid, 1.5 g (5.2 mmol) of 1-bromo-3-iodobenzene, 4.5 mL of a 2 mol/L aqueous solution of potassium carbonate, 20 mL of toluene, and 3 mL of ethanol, and the mixture was degassed by being stirred under reduced pressure. Furthermore, 52 mg (0.17 mmol) of tris(2-methylphenyl)phosphine (abbreviation: P(o-tolyl)₃) and 10 mg (0.043 mmol) of palladium(II) acetate (abbreviation: Pd(OAc)₂) were added to this mixture, and reacted under a nitrogen atmosphere at 80° C. for 14 hours. After the reaction, extraction with toluene was performed and the resulting organic layer was dried with magnesium sulfate. This mixture was subjected to gravity filtration and the obtained filtrate was purified by silica gel column chromatography (the developing solvent: hexane) to give 1.0 g of a target white solid (yield: 68%). The synthesis scheme of Step 1 is shown below.

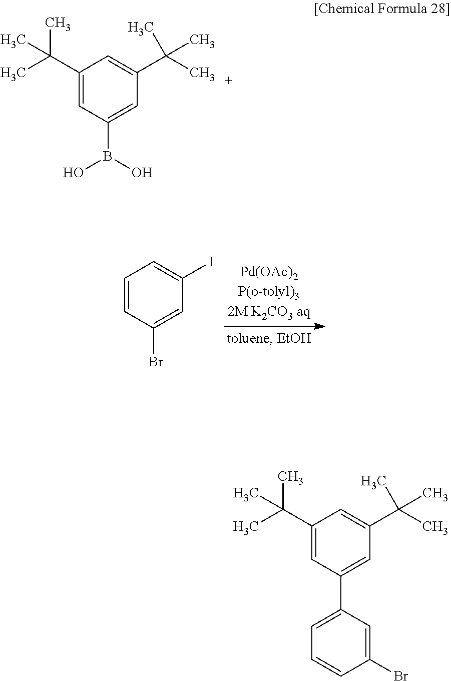 [SMILES: CC(C)(C)c1cc(B(O)O)cc(C(C)(C)C)c1 ; Brc1cccc(I)c1 ; CC(C)(C)c1cc(-c2cccc(Br)c2)cc(C(C)(C)C)c1]

### Step 2: Synthesis of 2-(3′,5′-di-tert-butylbiphenyl-3-yl)-4,4,5,5-tetramethyl-1,3,2-dioxaborolane

Into a three-neck flask were put 1.0 g (2.9 mmol) of 3-bromo-3′,5′-di-tert-butylbiphenyl, 0.96 g (3.8 mmol) of bis(pinacolato)diboron, 0.94 g (9.6 mmol) of potassium acetate, and 30 mL of 1,4-dioxane, and the mixture was degassed by being stirred under reduced pressure. Furthermore, 0.12 g (0.30 mmol) of 2-dicyclohexylphosphino-2′,6′-dimethoxybiphenyl (abbreviation: SPhos) and 0.12 g (0.15 mmol) of [1,1′-bis(diphenylphosphino)ferrocene]palladium(II) dichloride dichloromethane adduct (abbreviation: Pd(dppf)₂Cl₂·CH₂Cl₂) were added to this mixture to cause a reaction at 110° C. under a nitrogen atmosphere for 24 hours. After the reaction, extraction with toluene was performed and the resulting organic layer was dried with magnesium sulfate. This mixture was subjected to gravity filtration and the obtained filtrate was purified by silica gel column chromatography (the developing solvent: toluene) to give 0.89 g of a target yellow oil (yield: 78%). The synthesis scheme of Step 2 is shown below.

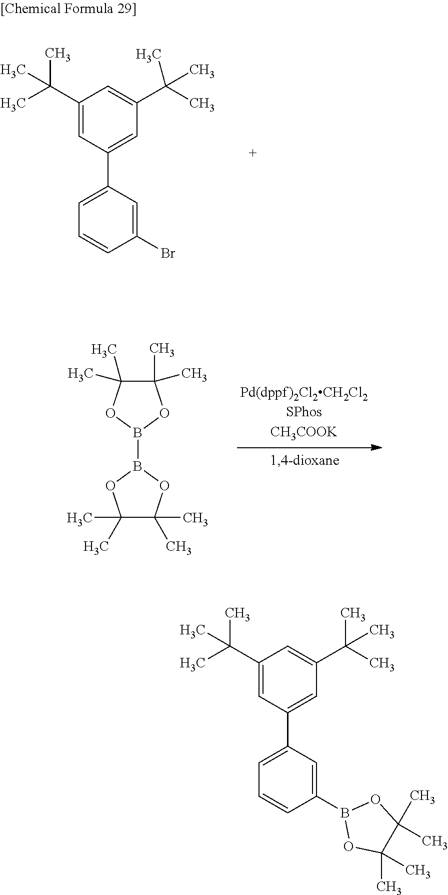

### Step 3: Synthesis of mmtBumBP-dmmtBuPTzn

Into a three-neck flask were put 0.8 g (1.6 mmol) of 4,6-bis(3,5-di-tert-butyl-phenyl)-2-chloro-1,3,5-triazine, 0.89 g (2.3 mmol) of 2-(3′,5′-di-tert-butylbiphenyl-3-yl)-4,4,5,5-tetramethyl-1,3,2-dioxaborolane, 0.68 g (3.2 mmol) of tripotassium phosphate, 3 mL of water, 8 mL of toluene, and 3 mL of 1,4-dioxane, and the mixture was degassed by being stirred under reduced pressure. To this mixture were added 3.5 mg (0.016 mmol) of palladium(II) acetate and 10 mg (0.032 mmol) of tris(2-methylphenyl)phosphine, and the mixture was heated and refluxed under a nitrogen atmosphere for 12 hours. After the reaction, extraction was performed with ethyl acetate and the obtained organic layer was dried with magnesium sulfate. This mixture was subjected to gravity filtration. The resulting filtrate was concentrated, followed by purification by silica gel column chromatography (the developing solvent: ethyl acetate:hexane=1:20) to give a solid. This solid was purified by silica gel column chromatography (the developing solvent: chloroform:hexane=5:1, which was then changed to 1:0). The obtained solid was recrystallized with hexane to give 0.88 g of a target white solid (yield: 76%). The synthesis scheme of Step 3 is shown below.

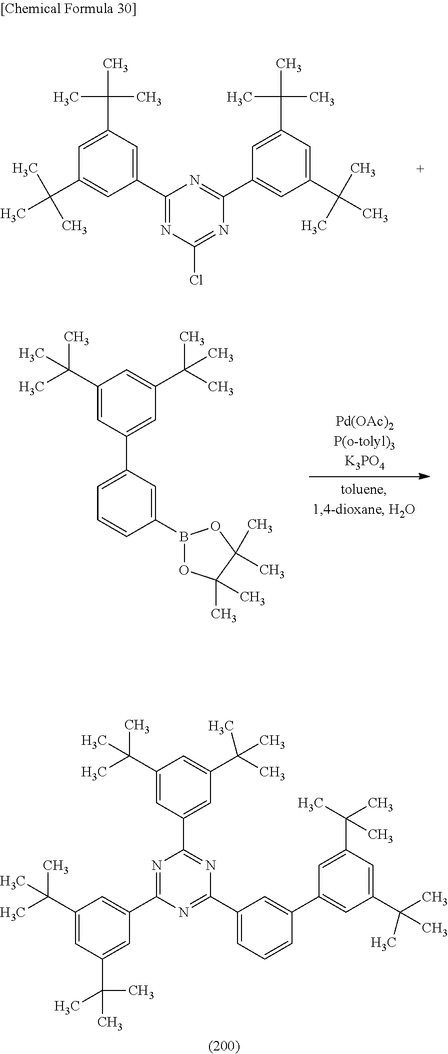

By a train sublimation method, 0.87 g of the obtained white solid was purified by sublimation by being heated under a pressure of 5.8 Pa at a temperature of 230° C. while an argon gas was made to flow. After the sublimation purification, 0.82 g of a target white solid was obtained at a collection rate of 95%.

The results of analysis by nuclear magnetic resonance spectroscopy (¹H-NMR) of the white solid obtained in Step 3 above are shown below. The results show that mmtBumBP-dmmtBuPTzn represented by Structural Formula (200) shown above was obtained by the above synthesis method.

¹H NMR (CDCl₃, 300 MHz): δ=1.42-1.49 (m, 54H), 7.50 (s, 1H), 7.61-7.70 (m, 5H), 7.87 (d, 1H), 8.68-8.69 (m, 4H), 8.78 (d, 1H), 9.06 (s, 1H).

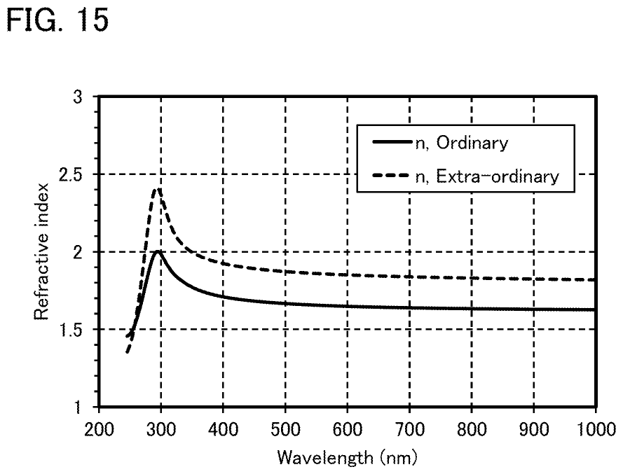

FIG. 15 shows the results of measuring the refractive index of mmtBumBP-dmmtBuPTzn, which was obtained by the above synthesis method, with a spectroscopic ellipsometer (M-2000U, produced by J.A. Woollam Japan Corp.). For the measurement, films obtained by depositing the materials of the respective layers to approximately 50 nm over a quartz substrate by a vacuum evaporation method were used. Note that FIG. 15 shows a refractive index for an ordinary ray, n, Ordinary, and a refractive index for an extraordinary ray, n, Extra-ordinary.

FIG. 15 shows that mmtBumBP-dmmtBuPTzn is a material with a low refractive index: the ordinary refractive index of mmtBumBP-dmmtBuPTzn in the entire blue emission region (from 455 nm to 465 nm) is in the range of 1.50 to 1.75, and the ordinary refractive index of mmtBumBP-dmmtBuPTzn for 633 nm is in the range of 1.45 to 1.70.

Similarly, the organic compounds represented by Structural Formulae (201) to (204) below were synthesized.

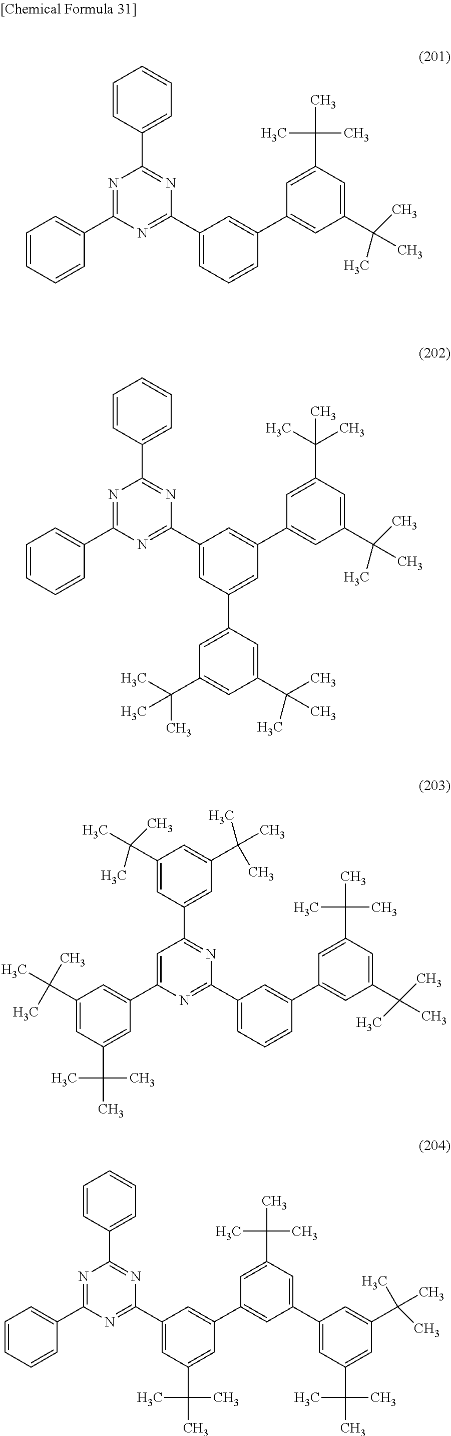

The results of analysis by nuclear magnetic resonance spectroscopy (¹H-NMR) of each organic compound are shown below.

### Structural Formula (201) 2-{(3′,5′-di-tert-butyl)-1,1′-biphenyl-3-yl}-4,6-diphenyl-1,3,5-triazine (Abbreviation: mmtBumBPTzn)

¹H NMR (CDCl₃, 300 MHz): δ=1.44 (s, 18H), 7.51-7.68 (m, 10H), 7.83 (d, 1H), 8.73-8.81 (m, 5H), 9.01 (s, 1H).

### Structural Formula (202) 2-(3,3″,5,5″-tetra-tert-butyl-1,1′:3′,1″-phenyl-5′-yl)-4,6-diphenyl-1,3,5-triazine (Abbreviation: mmtBumTPTzn)

¹H NMR (CDCl₃, 300 MHz): δ=1.44 (s, 36H), 7.54-7.62 (m, 12H), 7.99 (t, 1H), 8.79 (d, 4H), 8.92 (d, 2H).

### Structural Formula (203) 2-{(3′,5′-di-tert-butyl)-1,1′-biphenyl-3-yl}-4,6-bis(3,5-di-tert-butylphenyl)-1,3-pyrimidine (Abbreviation: mmtBumBP-dmmtBuPPm)

¹H NMR (CDCl₃, 300 MHz): δ=1.39-1.45 (m, 54H), 7.47 (t, 1H), 7.59-7.65 (m, 5H), 7.76 (d, 1H), 7.95 (s, 1H), 8.06 (d, 4H), 8.73 (d, 1H, 8.99 (s, 1H)).

### Structural Formula (204) 2-(3,3″,5′,5″-tetra-tert-butyl-1,1′:3′,1″-terphenyl-5-yl)-4,6-diphenyl-1,3,5-triazine (Abbreviation: mmtBumTPTzn-02)

¹H NMR (CDCl₃, 300 MHz): δ=1.41 (s, 18H), 1.49 (s, 9H), 1.52 (s, 9H), 7.49 (s, 3H), 7.58-7.63 (m, 7H), 7.69-7.70 (m, 2H), 7.88 (t, 1H), 8.77-8.83 (m, 6H).

The organic compounds described above each have an ordinary refractive index of higher than or equal to 1.50 and lower than or equal to 1.75 in a blue light emission range (455 nm to 465 nm) or an ordinary refractive index of higher than or equal to 1.45 and lower than or equal to 1.70 for 633-nm light, which is usually used for measurement of refractive indices.

### Example 2

In this example, samples imitating the light-emitting device of one embodiment of the present invention were evaluated by an electron spin resonance (ESR) method, and the results are described.

Methods for fabricating Sample 1, Comparative Sample 1, and Comparative Sample 2 of this example are described.

### (Fabrication Method of Sample 1)

At a ratio of 1 mol:8 mol, 12 tungsto(VI) phosphoric acid n-hydrate (abbreviation: H₃O₄₀PW₁₂, manufactured by FUJIFILM Wako Pure Chemical Corporation) and 4-diphenylaminodiphenylamine (DPA) were dissolved in N,N-dimethylformamide (DMF). The resultant solution was dripped onto a quartz substrate to form a film. The obtained substrate on which the film was formed was dried on a hot plate at approximately 150° C. to obtain Sample 1.

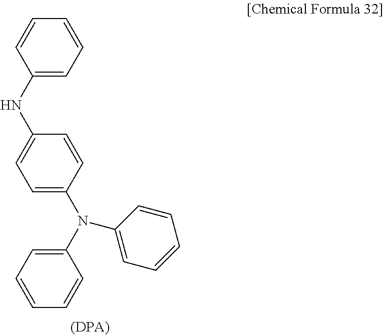 [SMILES: c1ccc(Nc2ccc(N(c3ccccc3)c3ccccc3)cc2)cc1]

H₃O₄₀PW₁₂ contained in Sample 1 is a heteropoly acid and DPA contained in Sample 1 is a secondary amine compound. That is, Sample 1 includes a mixed film of the heteropoly acid and the secondary amine.

### (Fabrication Method of Comparative Sample 1)

H₃O₄₀PW₁₂ was dissolved in DMF. The resultant solution was dripped onto a quartz substrate to form a film. The obtained substrate on which the film was formed was dried on a hot plate at approximately 150° C. to obtain Comparative Sample 1.

### (Fabrication Method of Comparative Sample 2)

DPA was dissolved in DMF. The resultant solution was dripped onto a quartz substrate to form a film. The obtained substrate on which the film was formed was dried on a hot plate at approximately 150° C. to obtain Comparative Sample 2.

The substrate of each of Sample 1, Comparative Sample 1, and Comparative Sample 2 was divided and put in a quartz tube, and electron spin resonance spectra (ESR spectra) were measured by an ESR method. An ESR spectrum derived from a vacant quartz tube was subtracted from each of the obtained ESR spectra to obtain values.

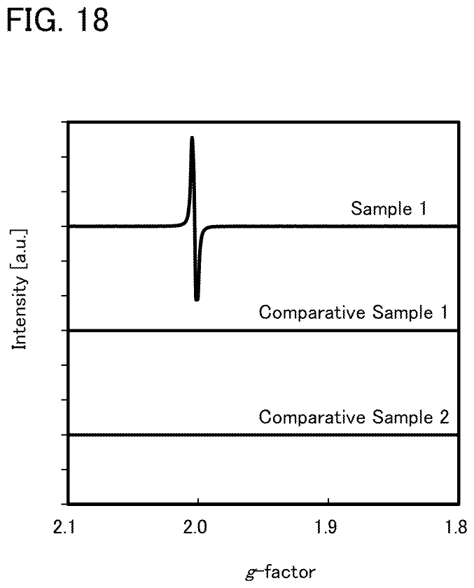

FIG. 18 shows the results of the ESR measurement of Sample 1, Comparative Sample 1, and Comparative Sample 2. FIG. 18 shows the ESR spectra of Sample 1, Comparative Sample 1, and Comparative Sample 2. In FIG. 18, the horizontal axis represents a g-factor and the vertical axis represents spin intensity [a.u.].

The measurement of the ESR spectra was performed with an electron spin resonance spectrometer JES FA300 (manufactured by JEOL Ltd.). The measurement was performed at room temperature under the conditions where the resonance frequency was approximately 9.2 GHz, the output power was 1 mW, the modulated magnetic field was 50 mT, the modulation width was 0.5 mT, the time constant was 0.03 sec, and the sweep time was 4 min. Magnetic field correction was performed with reference to the positions of Mn²⁺ third and fourth signals.

As a result of the measurement, a significantly intense signal was detected in the ESR spectrum of Sample 1. The g-factor calculated from the signal detected in the ESR spectrum of Sample 1 was approximately 2.00. This is the g-factor (g=2.00) derived from a singly occupied molecular orbital formed by the interaction between H₃O₄₀PW₁₂ and DPA. By contrast, no intense signal was detected at a g-factor of approximately 2.00 in the ESR spectrum of Comparative Sample 1 and the ESR spectrum of Comparative Sample 2.

Accordingly, it was found that the mixed film and mixture of the heteropoly acid and the secondary amine of embodiments of the present invention exhibited a significantly higher spin density at a g-factor of approximately 2.00 than each material not mixed. It was thus presumable that carriers were generated. Therefore, it was suggested that the mixed film of the heteropoly acid and the secondary amine of one embodiment of the present invention used for a hole-injection layer enables obtaining an element having a favorable hole-injection property. Furthermore, a hole-injection layer including the mixed film of the heteropoly acid and the secondary amine exhibits a signal at a g-factor of approximately 2.00 in its electron spin resonance spectrum measured by an ESR method.

Then, ultraviolet-visible absorption spectra of Sample 1, Comparative Sample 1, and Comparative Sample 2 were measured. The ultraviolet-visible absorption spectra were measured using an ultraviolet-visible spectrophotometer (V-550, and U-4100 manufactured by Hitachi, Ltd. for the thin films). The value was calculated using an absorbance (−log₁₀ [% T/(100−% R)]) obtained from the transmittance and the reflectance of the substrate and the sample. Note that % T represents the transmittance and % R represents the reflectance.

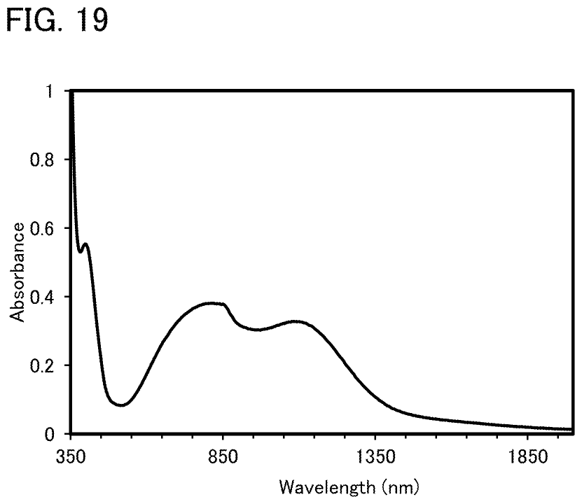

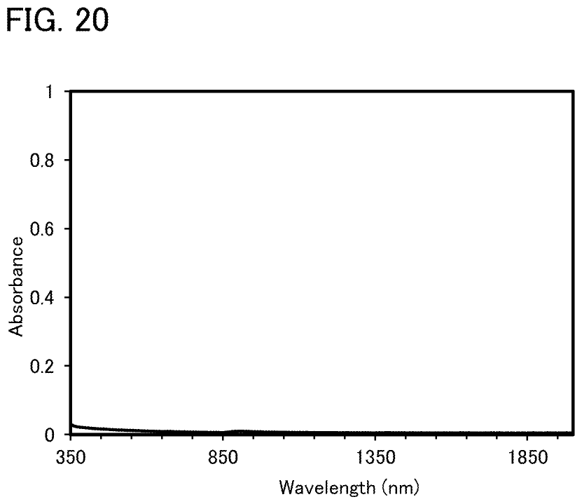

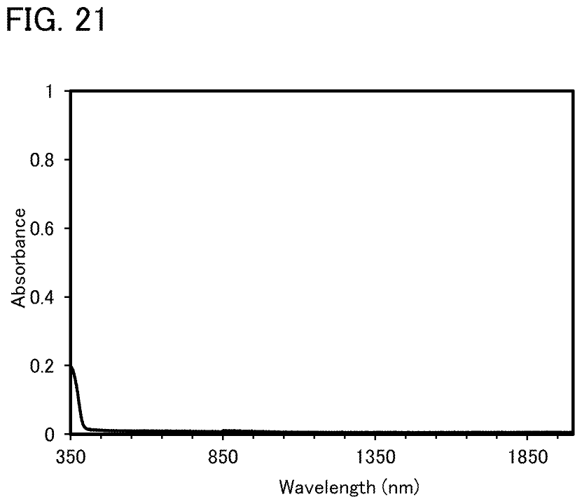

FIG. 19, FIG. 20, and FIG. 21 show the ultraviolet-visible absorption spectrum of Sample 1, that of Comparative Sample 1, and that of Comparative Sample 2, respectively. In FIG. 19 to FIG. 21, the horizontal axis represents a wavelength (nm) and the vertical axis represents an absorbance.

As shown in FIG. 19, absorption derived from intermolecular interaction (charge transfer transition) between H₃O₄₀PW₁₂ and DPA appeared in the range of around 400 nm to 1500 nm in the case of Sample 1. By contrast, as shown in FIG. 20 and FIG. 21, no intense absorption was detected in the above range in the cases of Comparative Sample 1 and Comparative Sample 2.

Note that the small peak at around 900 nm appearing in each of the ultraviolet-visible absorption spectra of Sample 1, Comparative Sample 1, and Comparative Sample 2 is distortion of the spectrum due to switching of the light source of the measurement device.

Accordingly, it was found that unlike each material not mixed, the mixed film and mixture of the heteropoly acid and the secondary amine of embodiments of the present invention exhibited intermolecular interaction (charge transfer transition)-induced absorption at around 400 nm to 1500 nm, which was significantly intense. This correlates with the aforementioned increase in ESR spin density. It was thus presumable that carriers were generated. Therefore, it was suggested that the mixed film of the heteropoly acid and the secondary amine of one embodiment of the present invention used for a hole-injection layer enables obtaining an element having a favorable hole-injection property. Furthermore, a hole-injection layer including the mixed film of the heteropoly acid and the secondary amine film exhibits absorption whose peak is located at greater than or equal to 400 nm and less than or equal to 1500 nm in measurement of an ultraviolet-visible absorption spectrum.

### REFERENCE NUMERALS

101: anode, 102: cathode, 103: EL layer, 111: hole-injection layer, 112: hole-transport layer, 113: light-emitting layer, 114: electron-transport layer, 115: electron-injection layer, 116: charge-generation layer, 117: p-type layer, 118: electron-relay layer, 119: electron-injection buffer layer, 120: hole-transport region, 121: electron-transport region, 400: substrate, 401: anode, 403: EL layer, 404: cathode, 405: sealing material, 406: sealing material, 407: sealing substrate, 412: pad, 420: IC chip, 601: driver circuit portion (source line driver circuit), 602: pixel portion, 603: driver circuit portion (gate line driver circuit), 604: sealing substrate, 605: sealing material, 607: space, 608: wiring, 609: FPC (flexible printed circuit), 610: element substrate, 611: switching FET, 612: current controlling FET, 613: anode, 614: insulator, 616: EL layer, 617: cathode, 618: light-emitting device, 623: FET, 730: insulating film, 770: planarization insulating film, 772: conductive film, 782: light-emitting element, 783: droplet discharge apparatus, 784: droplet, 785: layer, 786: layer, 788: conductive film, 951: substrate, 952: electrode, 953: insulating layer, 954: partition layer, 955: EL layer, 956: electrode, 1001: substrate, 1002: base insulating film, 1003: gate insulating film, 1006: gate electrode, 1007: gate electrode, 1008: gate electrode, 1020: first interlayer insulating film, 1021: second interlayer insulating film, 1022: electrode, 1024B: anode, 1024G: anode, 1024R: anode, 1024W: anode, 1025: partition, 1028: EL layer, 1029: cathode, 1031: sealing substrate, 1032: sealing material, 1033: transparent base material, 1034B: blue coloring layer, 1034G: green coloring layer, 1034R: red coloring layer, 1035: black matrix, 1037: third interlayer insulating film, 1040: pixel portion, 1041: driver circuit portion, 1042: peripheral portion, 1400: droplet discharge apparatus, 1402: substrate, 1403: droplet discharge means, 1404: imaging means, 1405: head, 1406: dotted line, 1407: control means, 1408: storage medium, 1409: image processing means, 1410: computer, 1411: marker, 1412: head, 1413: material supply source, 1414: material supply source, 1415: material supply source, 1416: head, 2001: housing, 2002: light source, 2100: robot, 2101: illuminance sensor, 2102: microphone, 2103: upper camera, 2104: speaker, 2105: display, 2106: lower camera, 2107: obstacle sensor, 2108: moving mechanism, 2110: arithmetic device, 3001: lighting device, 5000: housing, 5001: display portion, 5002: display portion, 5003: speaker, 5004: LED lamp, 5006: connection terminal, 5007: sensor, 5008: microphone, 5012: support portion, 5013: earphone, 5100: cleaning robot, 5101: display, 5102: camera, 5103: brush, 5104: operation button, 5120: dust, 5140: portable electronic apparatus, 5150: portable information terminal, 5151: housing, 5152: display region, 5153: bend portion, 5200: display region, 5201: display region, 5202: display region, 5203: display region, 7101: housing, 7103: display portion, 7105: stand, 7107: display portion, 7109: operation key, 7110: remote controller, 7201: main body, 7202: housing, 7203: display portion, 7204: keyboard, 7205: external connection port, 7206: pointing device, 7210: display portion, 7401: housing, 7402: display portion, 7403: operation button, 7404: external connection port, 7405: speaker, 7406: microphone, 9310: portable information terminal, 9311: display panel, 9313: hinge, 9315: housing

1-4. (canceled)

5. A light-emitting device comprising:
an anode;
a cathode; and
an EL layer between the anode and the cathode,
wherein the EL layer comprises a hole-transport region, a light-emitting layer, and an electron-transport region,
wherein the hole-transport region is between the anode and the light-emitting layer,
wherein the electron-transport region is between the cathode and the light-emitting layer,
wherein the hole-transport region comprises a heteropoly acid,
wherein the electron-transport region comprises an organic compound with an electron-transport property, and
wherein an ordinary refractive index of the organic compound with the electron-transport property is higher than or equal to 1.50 and lower than or equal to 1.75 for light with a wavelength of greater than or equal to 455 nm and less than or equal to 465 nm.

6. A light-emitting device comprising:
an anode;
a cathode; and
an EL layer between the anode and the cathode,
wherein the EL layer comprises a hole-transport region, a light-emitting layer, and an electron-transport region,
wherein the hole-transport region is between the anode and the light-emitting layer,
wherein the electron-transport region is between the cathode and the light-emitting layer,
wherein the hole-transport region comprises a heteropoly acid,
wherein the electron-transport region comprises an organic compound with an electron-transport property, and
wherein an ordinary refractive index of the organic compound with the electron-transport property is higher than or equal to 1.45 and lower than or equal to 1.70 for light with a wavelength of 633 nm.

7. The light-emitting device according to claim 5,
wherein the hole-transport region comprises the heteropoly acid and an organic compound comprising a π-electron rich aromatic ring or comprises the heteropoly acid and an organic compound comprising a π-electron rich heteroaromatic ring.

8. The light-emitting device according to claim 7,
wherein the organic compound comprising the π-electron rich aromatic ring and the organic compound comprising the π-electron rich heteroaromatic ring are each an aryl amine.

9. The light-emitting device according to claim 5,
wherein the hole-transport region comprises the heteropoly acid and a tertiary amine.

10. The light-emitting device according to claim 5,
wherein the heteropoly acid is phosphotungstic acid.

11. The light-emitting device according to claim 5,
wherein the hole-transport region comprises an arylsulfonic acid.

12. The light-emitting device according to claim 5,
wherein the hole-transport region comprises a hole-transport layer and a hole-injection layer,
wherein the hole-injection layer is in contact with the anode, and
wherein the hole-injection layer comprises the heteropoly acid and a secondary amine.

13-14. (canceled)

15. The light-emitting device according to claim 5,
wherein the light-emitting layer comprises an iridium complex.

16-17. (canceled)

18. The light-emitting device according to claim 15,
wherein the iridium complex is an iridium complex represented by Structural Formula (301).

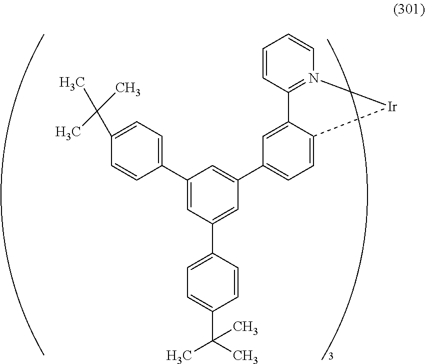

19. The light-emitting device according to claim 5,
wherein the organic compound with the electron-transport property comprises at least one nitrogen-containing six-membered heteroaromatic ring, two benzene rings, one or more aromatic hydrocarbon rings each having 6 to 14 carbon atoms, and a plurality of hydrocarbon groups forming a bond by sp³ hybrid orbitals, and
wherein the total number of carbon atoms forming the bond by the sp³ hybrid orbitals accounts for higher than or equal to 10% and lower than or equal to 60% of the total number of carbon atoms in a molecule of the organic compound with the electron-transport property.

20. The light-emitting device according to claim 5,
wherein the electron-transport region comprises an electron-transport layer and an electron-injection layer,
wherein the electron-injection layer is in contact with the cathode, and
wherein the electron-transport layer comprises the organic compound with the electron-transport property.

21. The light-emitting device according to claim 20,
wherein the electron-transport layer comprises a metal complex of an alkali metal or an alkaline earth metal.

22. The light-emitting device according to claim 21,
wherein the metal complex comprises a ligand comprising an 8-quinolinolato structure.

23. The light-emitting device according to claim 21,
wherein the metal complex is a metal complex of lithium.

24. (canceled)

25. The light-emitting device according to claim 20,
wherein the electron-injection layer comprises a heteroaromatic compound.

26. The light-emitting device according to claim 25,
wherein the heteroaromatic compound is 2-phenyl-9-[3-(9-phenyl-1,10-phenanthrolin-2-yl)phenyl]-1,10-phenanthroline.

27. The light-emitting device according to claim 20,
wherein the electron-injection layer comprises fluorine and sodium.

28. The light-emitting device according to claim 20,
wherein the electron-injection layer comprises barium.

29-33. (canceled)

34. The light-emitting device according to claim 5,
wherein the hole-transport region comprises a layer formed by applying and baking varnish comprising the heteropoly acid.

35. The light-emitting device according to claim 6,
wherein the hole-transport region comprises the heteropoly acid and an organic compound comprising a π-electron rich aromatic ring or comprises the heteropoly acid and an organic compound comprising a π-electron rich heteroaromatic ring.

36. The light-emitting device according to claim 35,
wherein the organic compound comprising the π-electron rich aromatic ring and the organic compound comprising the π-electron rich heteroaromatic ring are each an aryl amine.

37. The light-emitting device according to claim 6,
wherein the hole-transport region comprises a layer formed by applying and baking varnish comprising the heteropoly acid.
